(19) United States
(12) Patent Application Publication
(10) Pub. No.: US 2023/0338289 A1
(43) Pub. Date: Oct. 26, 2023

(54) METHODS AND COMPOSITIONS FOR TARGETED DELIVERY, RELEASE, AND/OR ACTIVITY

(71) Applicant: Jonathan M. Protz, Panama City (US)

(72) Inventor: Jonathan M. Protz, Panama City (US)

(21) Appl. No.: 17/796,427

(22) PCT Filed: Feb. 1, 2021
(86) PCT No.: PCT/US2021/016111

§ 371 Date: Jul. 29, 2022

Related U.S. Application Data
(60) Provisional application No. 63/205,293, filed on Nov. 27, 2020.
(60) Provisional application No. 63/205,294, filed on Nov. 27, 2020.
(60) Provisional application No. 63/204,924, filed on Nov. 2, 2020.
(60) Provisional application No. 63/204,592, filed on Oct. 13, 2020.
(60) Provisional application No. 63/103,352, filed on Jul. 30, 2020.
(60) Provisional application No. 63/102,624, filed on Jun. 22, 2020.
(60) Provisional application No. 63/101,931, filed on May 22, 2020.
(60) Provisional application No. 62/995,534, filed on Jan. 1, 0001.
(60) Provisional application No. 62/995,520, filed on Jan. 1, 0001.

(51) Int. Cl. A61K 9/127; A61K 47/54; A61K 31/7105

(52) U.S. Cl. CPC A61K 9/1272; A61K 47/549; A61K 31/7105

(57) ABSTRACT

Provided are compositions that include an active agent conjugated to a polymer strand and a carrier. In some embodiments, the polymer strand includes a monomer or unit, optionally a nucleotide sequence, that is modifiable to record one or more environmental conditions and/or a path in a volume experienced by the composition or that encodes a map for such a path for comparison to an actually-experienced path. Also provided are apparatuses for testing and/or recording one or more environmental conditions and/or traveled paths experienced by the compositions, methods for chemical recording of environmental sequences experienced by the compositions, kits that include the compositions and at least one reagent required to perform chemical recording of an environmental sequence experienced by the compositions, chemical recording devices that employ the compositions, drug delivery particles that include the compositions, and formulations of the compositions.

### CROSS REFERENCE TO RELATED APPLICATIONS

This application claims the priority benefit of U.S. Provisional Patent Application Ser. No. 62/995,520, filed Jan. 31, 2020; U.S. Provisional Patent Application Ser. No. 62/995,534, filed Jan. 31, 2020; U.S. Provisional Patent Application Ser. No. 63/101,931, filed May 22, 2020; U.S. Provisional Patent Application Ser. No. 63/102,624, filed Jun. 22, 2020; U.S. Provisional Patent Application Ser. No. 63/103,352, filed Jul. 30, 2020; U.S. Provisional Patent Application Ser. No. 63/204,592, filed Oct. 13, 2020; U.S. Provisional Patent Application Ser. No. 63/204,924, filed Nov. 2, 2020; U.S. Provisional Patent Application Ser. No. 63/205,293, filed Nov. 27, 2020; and U.S. Provisional Patent Application Ser. No. 63/205,294, filed Nov. 27, 2020. The disclosures of all of these provisional applications are incorporated herein by reference in their entireties.

### TECHNICAL FIELD

The presently disclosed subject matter relates primarily to the technology of compositions such as but not limited to pharmaceuticals and their delivery in animals and plants, but can also find application in any scenario requiring the location-specific release or application of compositions, chemicals, and/or other atoms, molecules, and/or small particles. Examples of the latter include, but are not limited to the selective patterning of surfaces in the processing or manufacture of semiconductors, electronic devices, and other micro devices.

### BACKGROUND

Targeted drug delivery is an area of active investigation and has been for several decades. Most approaches target chemically via cell-borne receptors or via genetics. Some use ex vivo stimulus such as heat or radio waves to drive spatially-localized release.

There are a variety of approaches to increase the specificity of the delivery or activity of a drug. Some approaches target delivery or activity on the basis of chemical interactions with chemical markers, receptors, or other such signatures exhibited by target cells. Others target delivery or activity with the assistance of an external aid that provides a signal for release. For example, a drug delivery vehicle can be sensitive to temperature and can release in localities where an external heat pack is applied. Alternatively or in addition, a drug delivery vehicle can be sensitive to radio-frequency radiation and can release in the proximity of a radiating antennae.

Approaches of the first type, which target chemical markers or other similar signatures, have the drawback of requiring some knowledge about the disease or cell type being targeted and also of being category-selective rather than location-selective. Approaches of the second type, which are activated or released by proximity to or in response to an external aid, are location-selective, but require the use of both the drug particle itself and the external aid.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list all possible combinations of such features.

In some embodiments, the presently disclosed subject matter relates to compositions comprising, consisting essentially of, or consisting of an active agent conjugated to a polymer strand, wherein the polymer strand comprises a monomer or unit, optionally a nucleotide sequence, that is modifiable to record one or more environmental conditions and/or a path in a volume experienced by the composition or that encodes a map for such a path for comparison to an actually-experienced path; and a carrier, wherein the carrier comprises a liposome, exosome, or other vehicle. In some embodiments, the nucleotide sequence is modified by regulating the addition and/or removal from the polymer strand to record the one or more environmental conditions and/or the path in the volume experienced at one or more of windows or probe tips of a micro-dialysis probed of any network or chain of micro-dialysis probes. In some embodiments, the composition further comprises a nuclease, a polymerase, or a terminal deoxynucleotidyl transferase (TdT) to append nucleotides to the polymer strand when experiencing the one or more environment conditions or path in the sequence of monomers of a polymer. In some embodiments, the polymer strand comprises a DNA strand and TdT is used to append nucleotides to the DNA strand when recording the one or more environmental conditions and/or the path in the volume. In some embodiments, the carrier is an environmentally sensitive liposome or exosome. In some embodiments, the composition makes use of the environmentally sensitive concentration, conformation, and/or level of activity of a catalyst to regulate appending monomers or units to the polymer strand when recording the one or more environmental conditions and/or the path in the volume.

In some embodiments, the presently disclosed subject matter relates to apparatuses for testing and/or recording of one or more environmental conditions and/or traveled paths experienced by the presently disclosed compositions. In some embodiments, the apparatuses comprise, consist essentially of, or consist of an interconnected network of in-line or concentric micro-dialysis probes that is supplied for its flow with a formulation that generates a polymer comprising a monomer sequence that is a function of the one or more environmental conditions and/or traveled paths experienced by the composition through a network, wherein one or more windows or tips of the micro-dialysis probes are exposed to the one or more different environmental conditions and/or traveled paths.

In some embodiments, the presently disclosed subject matter relates to methods for chemical recording of an environmental sequence experienced by a Fourier composition. In some embodiments, the methods comprise providing a presently disclosed Fourier composition and exposing the Fourier composition to one or more environmental conditions and/or paths in a volume experienced by the composition or that encodes a map for such a path for comparison to an actually-experienced path, whereby an environmental sequence experienced by the composition is recorded. In some embodiments, composition comprises, consists essentially of, or consists of a plurality of polymers and/or residuals thereof. In some embodiments, the method comprises removing monomers from the polymer or the plurality of polymers. In some embodiments, each member of the plurality of polymers comprises a sequence reflective of a basis function of a Fourier decomposition. In some embodiments, the presently disclosed methods further comprise recording a Fourier spectrum of an environmental signal by removing monomers from the plurality of polymers in an environmentally sensitive manner.

In some embodiments, the polymer comprises, consists essentially of, or consists of a nucleotide sequence, optionally a DNA sequence. In some embodiments, the polymer comprises, consists essentially of, or consists of an amino acid sequence.

In some embodiments, the presently disclosed subject matter relates to kits comprising a presently disclosed composition and at least one reagent required to perform a method for chemical recording of an environmental sequence experienced by the composition.

In some embodiments, the presently disclosed subject matter relates to uses of reverse transcription and/or mutagenesis in conjunction with a chemical recording technique to insert a record of an experienced environmental path into a polymer, optionally a nucleic acid.

In some embodiments, the presently disclosed subject matter relates to methods for consuming a monomer, block of monomers, or other such unit chain of a polymer in response to one or more environmental factors experienced by the monomer or the block of monomers or other such unit chain of a polymer to regulate a dosage of a therapeutic delivered to a target site, optionally a target site within an interconnected series of passages or volumes of an animal or a plant, optionally wherein the interconnected series of passages comprises a circulatory system of the animal or plant. In some embodiments, the methods comprise exposing the monomer, the block of monomers, or the other such unit chain of the polymer to the one or more environmental factors, wherein the exposing induces a modification of the monomer, the block of monomers, or the other such unit chain of the polymer. In some embodiments, the polymer is an RNA molecule that comprises a coding region and a junk tail region that follows or precedes the coding region, and further wherein the junk tail region serves as an environmentally path-sensitive fuse that controls a dose of a therapeutic delivered to a target and the coding region encodes a therapeutic agent or functions as a template for synthesis of a therapeutic, optionally wherein the therapeutic agent is a polypeptide, a DNA sequence, a DNA sequence that encodes a therapeutic RNA, or a DNA sequence that encodes a therapeutic polypeptide or protein. In some embodiments, the therapeutic agent is a therapeutic RNA, optionally an mRNA, an origami RNA, an interfering RNA, an RNA. In some embodiments, the therapeutic RNA encodes an immunogenic peptide or polypeptide. In some embodiments, the therapeutic agent is a polypeptide or a protein. In some embodiments, the monomer, block of monomers, or other such unit chain of the polymer comprises a junk fuse that functions to modulate consumption of the monomer, block of monomers, or other such unit chain of the polymer in a manner that is selective for a pre-determined path or target. In some embodiments, the coding region and junk fuse comprise, consist essentially of, or consist of a double-stranded DNA molecule, a single-stranded DNA molecule, and/or a DNA molecule that comprises one or more partially double-stranded regions and one or more single-stranded regions. In some embodiments, at least one of the one or more partially double-stranded regions comprises an RNA polymerase transcriptional start sequence, at least one of the one or more single-stranded regions comprises a junk tail region. In some embodiments, the coding region and the junk tail comprises an amino acid sequence, optionally a biologically active polypeptide, optionally a portion of which is therapeutically active, and further wherein the junk tail serves as the fuse, further optionally wherein the junk tail is associated with an enzyme that removes amino acids from the junk tail in an environmental path-sensitive manner.

In some embodiments, the presently disclosed subject matter relates to methods for targeted therapy. In some embodiments, the methods comprise administering to an animal or plant the composition of any one of claims 1-7, wherein the composition is encapsulated in a vehicle permits a reaction to take place while the composition traverses a circulatory system or some other such flow path or sequence of interconnected volumes of the animal or plant. In some embodiments, the vehicle is a liposome, an exosome, or other carrier the comprises a lipid bilayer. In some embodiments, the vehicle is a liposome comprising one or more pores in its lipid bilayer in order to make the liposome or the exosome permeable to the concentration of an ion and/or of one or more other environmental stimuli. In some embodiments, the pores are formed from a naturally occurring porin, an engineered porin, or any combination thereof integrated into the lipid bilayer.

In some embodiments, the presently disclosed subject matter relates to methods for using DNA as a template for producing an mRNA that comprises a coding region root or head and a junk region tail, wherein the junk region tail encodes a Fourier mode such that when the tail is attacked by a nuclease, optionally, an RNase, that removes units from the junk region tail with a removal rate that is sensitive to environment and to the type of unit being removed or the types of units in the vicinity of a removal site, and further wherein a mathematical vector dot product or inner product is accomplished and the time average removal rate, which is also the time until the coding region is broken, is represented as being sensitive to the inner product of the time-evolution of an environment and the spatial sequence of monomers in the junk region tail.

In some embodiments, the presently disclosed subject matter relates to methods for synthesizing from a fused RNA via reverse transcription a sticky-end cDNA that is ligatable one or more nucleotide sequences strands that code for the synthesis of mRNA. In some embodiments, the methods comprise providing a nucleotide sequence encoding a sticky-end cDNA and a reverse transcriptase under conditions sufficient to synthesize a sticky-end cDNA from the fused RNA. In some embodiments, polymers in general are used rather than specifically nucleic acid polymers. In some embodiments, the tail removed from the RNA or the DNA in the environmentally sensitive fashion is synthesized using a TdT noisy chemical recording technique.

In some embodiments, the presently disclosed subject matter relates to kits comprising a presently disclosed composition and at least one reagent required to employ the composition to regulate dosing of the active agent or synthesis of a polypeptide encoded by the polymer strand.

In some embodiments, the presently disclosed subject matter relates to methods for synthesizing a double-stranded DNA that encodes an mRNA comprising a coding head and a Fourier mode tail. In some embodiments, the methods comprise reverse transcribing one or more cDNA sticky-ended pieces of the mRNA, and thereafter ligating the sticky-ended pieces together to form the double-stranded DNA.

In some embodiments, the presently disclosed subject matter relates to formulations for chemical recording and DNA editing. In some embodiments, the formulations comprise, consist essentially of, or consist of a DNA plasmid that comprises one or more genes required to synthesize an environmentally sensitive fusing of protein synthesis scheme described above and that also contains the genes required for expression of the various proteins and nucleic acid chains used in the reaction (in addition to or beyond those already provided in an appropriate cell).

In some embodiments, the presently disclosed methods further comprise adding a tail to DNA by TdT or another tailing reaction, optionally with sticky ends and T4 DNA ligase, after a coding region, wherein the coding region lacks a terminator between the region and the tail, such that transcription by RNA polymerase runs off the tail and the tail length thusly affects the rate of RNA polymerase recirculation and, therefore, the rate of transcription and translation, and, therefore an amount of polypeptide, or RNA, or reverse transcription product encoded by the coding region.

In some embodiments, the presently disclosed subject matter relates to chemical recording devices or formulations that write a polymer strand by regulating the addition of monomers to a polymer via a valve-like mechanism and that is used to record one or more environments experienced at one or more of windows or probe tips of a micro-dialysis probe or a network or chain of micro-dialysis probes. In some embodiments, the chemical recording device or formulation records a sensed environment or path in a sequence of monomers of a DNA strand. In some embodiments, the regulating makes use of TdT to append nucleotides to a DNA strand when recording the sensed environment or path in the sequence of monomers of a polymer. In some embodiments, the regulating makes use of environmentally-sensitive liposomes as a valve that regulates the process of appending monomers or units to a polymer or other chain or strand when recording the sensed environment or path in the sequence of monomers of a polymer or units of some chain. In some embodiments, the regulating makes use of an environmentally-sensitive concentration, conformation, and/or level of activity of a catalyst as a valve to regulate appending monomers or units to a polymer or other chain or strand when recording the sensed environment or path in the sequence of monomers of a polymer or units of some chain.

In some embodiments, the presently disclosed subject matter relates to apparatuses for chemical recording of environments or traveled paths. In some embodiments, an apparatus of the presently disclosed subject matter comprises, consists essentially of, or consists of an interconnected network of in-line and/or concentric micro-dialysis probes that is supplied for its flow with a formulation that generates a polymer, a monomer sequence of which is a function of a path traveled through the network, the windows, or probe tips of the probes when exposed to different environments. In some embodiments, the apparatus is structured to record a sensed environment and/or path in nucleotides of a DNA strand, amino acids of a polypeptide, and/or monomers of a sugar polymer.

In some embodiments, the presently disclosed subject matter relates to use of reverse transcription and/or mutagenesis in conjunction with a chemical recording method of any one of the preceding claims to insert a record of an experienced environmental path into a nucleic acid, optionally a DNA. In some embodiments, the nucleic acid is a cDNA.

In some embodiments, the presently disclosed subject matter relates to use of mRNA translation in conjunction with a chemical recording method of any one of the preceding claims to regulate synthesis of a protein in such a way that the synthesis is affected by experienced environmental path. In some embodiments, the translating exhibits phenotypic evolution with changes in environment. In some embodiments, the translating employs a nucleic acid and enzyme system that exhibits genotype evolution with changes in environment.

In some embodiments, the presently disclosed subject matter relates to methods for randomly removing segments of DNA from a plasmid or piece of linear DNA while simultaneously adding cDNA synthesized through a reverse transcription process that is affected by experienced environmental path, wherein an approximate size of the DNA is relatively constant over time but its composition evolves with changing environment.

In some embodiments, the presently disclosed subject matter relates to methods for employing one or more naturally occurring porins and/or engineered porins to make a liposome permeable to an ion, the concentration of which regulates a chemical recorder of genetic memory mechanism.

In some embodiments, the presently disclosed subject matter relates to use of a block of DNA as a template for a piece of mRNA that features a coding region root or head and a junk region tail, wherein the junk region tail encodes a Fourier mode such that when the tail is acted upon by a enzyme, optionally an RNase, that removes units from it with a removal rate that is sensitive to environment and to the type of unit being removed and/or the types of units in the vicinity of the removal site, a mathematical vector dot product or inner product is accomplished and the time average removal rate, which is also the time until the coding region is broken, is represented as being sensitive to the inner product of the time-evolution of an environment and/or one or more other environmental stimuli.

In some embodiments, the presently disclosed subject matter relates to drug delivery particles comprising a composition as disclosed herein.

In some embodiments, the presently disclosed subject matter relates to methods for drug delivery. In some embodiments, the methods comprise, consist essentially of, or consist of administering to a subject in need thereof an effective amount of a presently disclosed drug delivery particle. In some embodiments, the drug delivery particle employs correlation of a local environment in a circulatory system of the subject, or of a sequence of such environments, against a map embodied in its parameters in order to establish and respond to one or more locations of the drug delivery particle in the subject. In some embodiments, the drug delivery particle employs correlation of a local environment in which it is located, or of a sequence of such environments, against a map embodied in its parameters in order to establish and respond to its location within the subject.

In some embodiments, the presently disclosed subject matter relates to use of a microarray, bio-chip, bio-MEMS device, well plate array, and/or other apparatus to test a presently disclosed composition with respect to sensitivity to its environment.

In some embodiments, the presently disclosed subject matter relates to a microarray, bio-chip, bio-MEMS device, and/or well plate array structured to test a particle for sensitivity to environment or sequence of environments that the particle experiences. In some embodiments, the microarray, bio-chip, or bio-MEMS device is structured to populate elements therein with material and/or chemical contents with material and/or chemical properties and other such metrics drawn from or patterned after a conventional surgical testing apparatus known as a ‘synthetic cadaver’, or any other such similar apparatus, or any database the contents of which are similar in concept to sampling the various environments of such an apparatus, or any database the contents of which are suitable for designing such an apparatus, or any part of such a database or any database that could form part of such a database, on a chip or similar apparatus. In some embodiments, the bio-chip or bio-MEMS device is structured in the form of a synthetic cadaver on a chip.

In some embodiments, the presently disclosed subject matter relates to particles comprising, consisting essentially of, or consisting of a polypeptide or long-chain polymer or other strand of material with an environmentally-sensitive conformation conjugated to or associated with a drug moiety or active region that activates under different environmental conditions or sequences of environmental conditions. In some embodiments, the conformation depends on the sequence of environmental conditions. In some embodiments, vehicles containing or bearing one or more particles with TERCOM-like or DSMAC-like behavior are encased by a film such as a porous lipid bilayer or some other material film. In some embodiments, the vehicles are encased by a film such as a porous lipid bilayer or some other material film. In some embodiments, the vehicles are tethered or connected but not encased by any additional material, meaningful resistance to diffusion, to the extent it exists, occurring simply due to the radial nature of diffusion out of an interstitial volume. In some embodiments, the interstitial volume serves as a capacitive layer that averages the rate of release from the individual particle types.

Thus, it is an object of the presently disclosed subject matter to provide compositions and methods for targeted delivery of therapeutics.

An object of the presently disclosed subject matter having been stated hereinabove, and which is achieved in whole or in part by the presently disclosed subject matter, other objects will become evident as the description proceeds when taken in connection with the accompanying Figures as best described herein below.

### BRIEF DESCRIPTION OF THE FIGURES

FIG. 1 is an exemplary apparatus that can be used to test sensitivities of the presently disclosed compositions to path.

FIG. 2 is a schematic diagram showing a nuclease with environmental and substrate sensitivity consumes protective tail and reaches active therapeutic site faster when reaction mixture travels along non-target paths, causing larger dose of therapy to be delivered to target sites than to non-target sites.

FIG. 3 is a schematic diagram of an exemplary implementation of the presently disclosed subject matter.

### DETAILED DESCRIPTION

The co-inventor's previous work in this area, including but not limited to U.S. Patent Application Publication No. 2009/0275031 and PCT International Patent Application Publication No. WO 2019/217601, each of which is incorporated herein in its entirety, focused on the development of nanoparticles capable of sensing and retaining a memory of their environment. Droplets of suspensions of DNA-charged liposomes and enzymes stitched within themselves DNA chains that noisily recorded the temperature history experienced by the droplets. The presently disclosed subject matter expands on these areas in several respects, including but not limited to liposome-encapsulated nucleic acids that comprise a removable tail sensitive to environmental path, use of a “fuse” to regulate doses of compounds to be delivered, liposome-encapsulated nucleic acids comprising a tail and/or chemical addition to a protein and/or other polymer that is sensitive to environmental path, employing the presently disclosed compositions for gene editing, shape-changing polymers such as but not limited to environmentally sensitive polypeptides, diffusion-based release of compositions through charged liposomes, and apparatuses and formulation designs applicable to the compositions and methods disclosed herein. Particularly, as disclosed herein, the compositions and methods of the presently disclosed subject matter take advantage of TERCOM (i.e., environmental path sensitivity) employing Fourier modes and/or monomer removal and/or addition/fusing.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a” “an”, and “the” refer to “one or more” when used in this application, including in the claims. For example, the phrase “a cell” refers to one or more cells, including a plurality of the cells. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

With respect to the terms “comprising”, “consisting of”, and “consisting essentially of”, where one of these three terms is used herein, the presently disclosed and claimed subject matter can include the use of either of the other two terms. For example, in some embodiments, the presently disclosed subject matter relates to compositions comprising antibodies. It would be understood by one of ordinary skill in the art after review of the instant disclosure that the presently disclosed subject matter thus encompasses compositions that consist essentially of the antibodies of the presently disclosed subject matter, as well as compositions that consist of the antibodies of the presently disclosed subject matter.

The term “subject” as used herein refers to a member of any invertebrate or vertebrate species. Accordingly, the term “subject” is intended to encompass any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals)), and all Orders and Families encompassed therein.

The compositions and methods of the presently disclosed subject matter are particularly useful for warm-blooded vertebrates. Thus, the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A goal of the presently disclosed subject matter is to allow the delivery of drugs to specific points within the body, such as to the vicinity of diseased tissue, without requiring the use or knowledge of chemical or biological markers uniquely associated with the targeted tissue and without requiring the involvement of external apparatus such as heating elements or radio transmitters. A core feature of the presently disclosed subject matter is one of targeting delivery using a particle's own estimates of particle location within the body to target drug release at points specified on the basis of off-line medical imaging. Thus, in some embodiments it is closely related to the terrain correlation mapping or terrain contour matching (TERCOM) techniques used in aircraft navigation.

In some embodiments, particles, delivery vehicles, and formulations of the presently disclosed subject matter can estimate their own location within the body by correlating vectors of sensed environmental variables (e.g., temperature, pressure, salinity, sugar levels, pH, etc.) or their time history against a map release drugs or other chemicals at targeted sites on the basis of this location estimate. This approach eliminates the reliance upon ex vivo navigation aids and cell markers. This specificity of location is accomplished by tailoring material properties of an inanimate material particle or the genetics of a synthetic biological life form.

Thus, in some embodiments the particles, delivery vehicles, or formulations are sensitive to environmental stimuli in the various different environments within a subject's body, particularly within different systems of the subject's body as well as different locations within any given system (including but not limited to the circulatory system). Exemplary environmental stimuli can include, but are not limited to variations in temperature, pressure, salinity, sugar levels, pH, etc. As such, in some embodiments a particle, delivery vehicle, or formulation of the presently disclosed subject matter comprises a structure such as but not limited to a liposome or nanoparticle that alters its composition and/or conformation in response to variations in temperature, pressure, salinity, sugar levels, pH, etc. that it experiences as it traverses the subject's circulatory system. Exemplary delivery vehicles that can alter their compositions and/or conformations in response to variations in vivo environmental stimuli include, but are not limited to those disclosed in U.S. Pat. No. 7,780,979 (temperature-sensitive hydrogels), the entire disclosure of which is incorporated by reference.

Furthermore, polymers can be formed into gels by dispersing them into a solvent such as water. In certain embodiments, polysaccharides and polypeptides and other polymers can be fashioned to release microparticles and/or a therapeutic agent present in the microparticles upon exposure to a specific triggering event such as pH (see e.g., Heller et al., 1988; Peppas, 1993; Doelker, 1993. Representative examples of pH-sensitive polysaccharides include carboxymethyl cellulose, cellulose acetate trimellilate, hydroxypropylmethylcellulose phthalate, hydroxypropyl-methylcellulose acetate succinate, chitosan and alginates.

Similarly, polysaccharides and polypeptides and other polymers can be fashioned to be temperature sensitive (see e.g., Okano, 1995; Hoffman et al., 1993; Hoffman, 1988; Hoffman, 1987. Representative examples of thermogelling polymers, such as poly(oxyethylene)-poly(oxypropylene) block copolymers (e.g., PLURONIC F127 from BASF Corporation, Mount Olive, New Jersey, United States of America), and cellulose derivatives. Paclitaxel microspheres having lower, traditional loadings have been incorporated into a thermoreversible gel carrier (PCT International Patent Application Publication No. WO 2000/066085).

Exemplary polysaccharides include, without limitation, hyaluronic acid (HA), also known as hyaluronan, and derivatives thereof (see e.g., U.S. Pat. Nos. 5,399,351; 5,266,563; 5,246,698; 5,143,724; 5,128,326; 5,099,013; 4,913,743; and 4,713,448), including esters, partial esters and salts of hyaluronic acid. For example, an aqueous solution of HA having a non-inflammatory molecular weight (greater than about 900 kDa) and a concentration of about 10 mg/ml would be in the form of a gel. The aqueous solution may further comprise one or more excipients that serve other functions, such as buffering, anti-microbial stabilization, or prevention of oxidation. Microspheres made from, for example, 70% paclitaxel loaded poly(L-lactide), MW=2000, may be incorporated into a 10 mg/ml HA gel as follows. HA, MW=1 MDa, is dissolved in water to a concentration of 20 mg/ml and microparticles are dispersed in water to a concentration in the range of 0.02 to 20 mg/ml. The two phases are combined in equal volumes by mixing (e.g., syringe mixing, using two interconnected luer lok syringes between which the liquids are passed back and forth fifty times), such that the microparticles are evenly distributed throughout the mixture, which has a concentration of 10 mg/ml HA and between 0.1 and 10 mg/ml microparticles, equivalent to 0.07 and 7 mg/ml paclitaxel in a gel carrier.

Also, other polymeric carriers can be fashioned which are temperature sensitive are known. See e.g., Chen et al., 1995; Johnston et al., 1992; Tung, 1994; Harsh & Gehrke, 1991; Bae et al., 1991; Dinarvand & D'Emanuele, 1995a; Yu & Grainger, 1993a; Zhou & Smid, 1993; Yu & Grainger, 1993b; Kim et al., 1992; Bae et al., 1991; Kono et al., 1994; Yoshida et al., 1994; Okano et al., 1995; Chun & Kim, 1996; D'Emanuele & Dinarvand, 1995b; Katono et al., 1991; Gutowska et al., 1992; Palasis & Gehrke, 1992; Paavola et al., 1995.

Representative examples of thermogelling polymers, and their gelatin temperature (LCST; ° C.) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly (N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water soluble polymers such as acrylmonomers (e.g., acrylic acid and derivatives thereof such as methylacrylic acid, acrylate and derivatives thereof such as butyl methacrylate, acrylamide, and N-n-butyl acrylamide). Other representative examples of thermogelling polymers include cellulose ether derivatives such as hydroxypropyl cellulose, 41° C.; methyl cellulose, 55° C.; hydroxypropylmethyl cellulose, ° C.; and ethylhydroxyethyl cellulose, and Pluronics such as F-127, 10-15° C.; L-122, 19° C.; L-92, 26° C.; L-81, 20° C.; and L-61, 24° C.

In some embodiments, composite shells of polymer or lipid bilayer penetrated with a chowder and/or fluid mosaic of conformation-changing particles, each sensitive to different stimuli and with different thresholds, are tailored to change porosity after exposure to a path-specific sequence of environmental shifts.

In some embodiments, encapsulated droplets of environmentally-responsive suspensions might be engineered to release indicators whenever a specific environmental sequence is experienced.

Other embodiments are also envisioned. All share the features that they are composed of mixtures of materials with different sensitivities to environment and that one or more of the parameters of these different materials is selected in such a way as to ensure that when the composition experiences a particular sequence of shifts in its environment it responds in a specific, predictable manner, and when it does not, it does not.

Compositions can include within them polymer vehicles, proteins, computational DNA, engineered cells, biodegradable nanoparticles, etc., can substitute; all are common in drug delivery research and have been explored by other investigators in a variety of comparable applications.

An advantage of the presently disclosed subject matter when applied to the medical applications is that it allows locally-selective drug delivery without requiring prior knowledge of disease markers or the genetics of the patient or disease, allowing the use instead of information gathered through medical imaging.

Also, the approaches disclosed herein, being stochastic in nature, can allow the development of libraries of delivery particles that are each selective for specific locations within the body, based on environmental variables, since each particle effectively carries its own internal map.

Thus, in some embodiments the presently disclosed subject matter focuses on targeted drug delivery mechanisms wherein internal estimates of particle location within a body or other interconnected network of volumes is used to target release of drugs or chemicals or the expression of genes or other such signatures at points specified on the basis of off-line medical imaging.

In some embodiments, the subject matter disclosed herein relates to mechanisms for estimating location within the body from vectors of sensed environmental variables (e.g., temperature, pressure, salinity, sugar levels, pH, etc.) or their trailing averages. Particularly, in some embodiments it uses particles or formulations that estimate their own location within the body by correlating such vectors of sensed environmental variables (e.g., temp., pressure, salinity, sugar levels, pH, etc.) against a map and release their drug at targeted sites on the basis of this location estimate.

In some embodiments, shells of polymer or lipid bilayer penetrated with a chowder of conformation-changing particles, each sensitive to different stimuli and with different thresholds, might be tailored to change porosity after exposure to a path-specific sequence of environmental shifts; or, encapsulated droplets of environmentally-responsive suspensions might be engineered to release indicators whenever a specific environmental sequence is experienced.

Registration of environmental terrain with anatomical location can be accomplished off-line through a multi-factor regression of blood samples, etc. drawn from a set of representative individuals at consistent locations throughout the body and under various contexts (e.g., wake. sleep, etc.), all collapsed onto a single non-dimensional parametric model representing the generalized morphology of the body or circulatory system. Traverse of the body can be modeled by a stochastic, discrete-state, continuous-transition Markov process with vascular branch or region as state.

Delivery can be modeled as brute force decryption, with the targeted capillary as message, the location-sensitive particle as cryptogram, the trailing history of branch environments as trial key, vascular circulation as a random cycling of trial keys, and release as a successful decryption.

Particle design can then be cast as a problem of robust optimization of material parameters with a goal of balancing type I and II errors in release.

Targeted drug delivery is an area of active investigation and has been for several decades. Most approaches target chemically via cell-borne receptors or via genetics. Some use ex vivo stimulus such as heat or radio waves to drive spatially-localized release. In the first case, drug-laden vehicles formed from lipid bilayers (liposomes) with the special property that they become more porous when exposed to a temperature above some threshold. By using an external heat pack or heating element to apply heat or cold locally to some specific area of the body, these can be stimulated to release their drugs in this local region. In the second case, DNA strands tagged with gold atoms can be caused to open when exposed to radio waves of specific frequency, making the exposed regions available for transcription. By focusing radio waves from an external source on some specific region of the body, these can be cause to be available for transcription in a therapeutic capacity at that location.

There are many other potential embodiments like these. In some embodiments, they all have in common the feature that they rely on some means of stimulating drug release that is outside of the normal operation of the human body

As set forth herein, the drug delivery particles are designed specifically to require the application of no external or unnatural stimulus and to be highly selective for a specific locale within the body.

A particular mathematical problem of interest is for the case of a continuous-transition, discrete-state Markov chain of with a single chain of finite extent and no periodic states; this is ergodic with stable long-run probabilities (if the chain is very large or infinite, it can be divided into a set including the initial state within which there is a high probability or remaining over some arbitrary time horizon, and a set which there is a low probability of entering). Such a chain is representative of the movement of blood and the particles it carries through the circulatory system. At each juncture, there is some probability of taking one branch over the other, but the system is closed so that every juncture can eventually be visited after a long enough period of time. A particle or system can move through this network with the particle or system having some structure that is capable of causing changes in its internal state (e.g., its porosity, whether a sensing mechanism is on, etc.) or external state (e.g., charge) when exposed to a specific sequence of environmental conditions.

In this case, the control problem is differentiated from traditional deterministic control in the face of random uncertainty by the fact that the actuator authority will never be sufficient to drive the system along a specific desired trajectory. Instead, the best that can be hoped for is to influence the transition probabilities with the goal of having the statistics of the system's trajectory be as close as possible to some desired ideal (e.g., maximize the frequency some specific desirable site is visited or minimize the time spent over some undesirable site).

A number of control approaches can be considered, with each providing some parameterized mapping between the sensed variables and adjustments to the actuators.

The primary controller embodiment of interest is a multilayer perceptron network composed of a bank of thresholding functions that take as their input weighted sums of the sensed variable and that are fed into an AND function (everything must pass); in a related embodiment, they are summed after weighting and fed into another threshold function (some fraction must pass); In some embodiments they are passed into some other such mechanism. In all of these embodiments, the parameters to be optimized are the weights of the sums or, where appropriate, the threshold levels (generally, these would be normalized against the weights). Note that this mechanism is fundamentally a collection of linear classifiers that together are used to select an action, and the optimization problem is one of specifying each classifier.

The specific optimization problem is then structured as choosing the parameters (e.g., the slopes of the linear classifiers) in a way that balances the maximizing of some goal function, for example the fraction of time spent near desired location, while minimizing some penalty function, for example the non-uniformity of the distribution of time spent over undesirable locations, while also satisfying some set of constraints (e.g., expected cumulative net energy consumption remains always below some level).

Standard techniques familiar to those skilled in the state of the art of machine learning and optimization can be used to solve the optimization problem using theory or heuristics in a manner that is optimal or boundedly suboptimal. These can include the development and backtesting of a support vector machine using a subset of gathered experimental data. In the specific instance of designing for a drug delivery particle, blood tests can be taken at a variety of locations and a subset of these used to compute the parameters of the support vector machine, which is then tested against the remaining data.

Thus, described herein are particles, formulations, and devices that deliver to one or more locations in a flow network (or other interconnected set of volumes or spaces including but not limited to the body of a subject) a molecule or chemical composition tailored to each point of delivery. The particle, formulation, or device can also be used as a chemical recorder that records the spatial history of the environment experienced by the particle, formulation, or device as it traverses a path.

In some embodiments, a formulation for the synthesis of a polymer is prepared that exhibits the feature that when passed through two or more different spatial pathways, each of which features a different sequence of local chemical environments, the formulation synthesizes polymers having different monomer sequences, with the sequences encoding information about the spatial environment which was traversed, and an apparatus is prepared which features two or more differing spatial pathways for said formulation to traverse, with the differing pathways featuring local chemical or physical environments that vary along each path differently. This formulation and apparatus, and their operation, are described herein detail, which provides a detailed experimental plan which describes the formulation, the apparatus, and the procedural steps for performing an experiment with them. The embodiments described herein, particularly in the EXAMPLES, are understood to be merely exemplary embodiments.

In some embodiments, the above-described formulation is encapsulated in a liposome or other vehicle (e.g., exosomes, ethosomes, etc.) that features pores and/or passive diffusion channels (or similar such features), which allow those ions that are sensed by the presently disclosed formulations to pass into the liposome/vehicle, where they can affect the recording reaction. In some embodiments, the pores and/or passive diffusion channels allow only small ions such as one or more of those of Ca, Co, Mg, Zn, Na, etc. to pass freely, but do not allow larger substances such as but not limited to nucleotides and/or other recording units (e.g., sticky-ended DNA segments) to pass. In some embodiments, not only can small ions such as those listed pass, but also nucleotides (and/or other recording units). In embodiments, however, enzymes used for polymer synthesis (e.g., TdT and/or T4 DNA ligase) and the assembled polymer chains cannot pass.

In some embodiments of the above, the TdT-based formulation described herein is encapsulated in a liposome, exosome, or ethosome.

In some embodiments, liposomes (e.g., proteoliposomes) are described that allow the passage of ions that are used to regulate the formation of a polymer. Non-limiting examples of vehicles used to contain an environmentally sensitive reaction mixture of the presently disclosed subject matter used for polymer synthesis are described herein. Some of those embodiments are described as liposomes that employ trans-membrane proteins to provide a way of passing ions which are used in the regulation of the polymer formation reaction (e.g., Cobalt), including some that use TRPM-type proteins to create the permeability. Exemplary such proteins include, but are not limited to porins and other passive transport proteins used to create pores in cell walls. In some embodiments, which are not intended to be restrictive, as the presently disclosed subject matter encompasses any liposome-type vehicles that use or can be modified to use porins or other proteins, molecules, or assemblages of molecules that can create passive transport pores in their outer surfaces (e.g., a liposome bilayer) that allows the interior environment of the vehicle to reflect in some way the outer environment that surrounds the liposome, the protein can be one that described in Kobayashi et al., 1982 or Macdonald et al., 2002, and the process of liposome preparation is that described in Kobayashi et al., 1982 or Macdonald et al., 2002. In some embodiments, liposomes with porins can be prepared as in Macdonald et al., 2002.

In some embodiments, a composition of the presently disclosed subject matter can be made unencapsulated (in a test tube or slug) by adding a DNA of the sort described in that filing (with Fourier tails) and exonuclease T to the reagent mixture from the Central Dogma experiment kit sold by miniPCR (Amupuls, and then running the experiment in the otherwise same way, although regulating the ion concentration (e.g., the concentration of Co⁺²). Alternatively or in addition, an encapsulated version can be made by encapsulating the test tube reaction mixture in a liposome.

In some embodiments, a sensitive particle as described herein has the feature that information is recorded by incorporating it into DNA through a process that involves generating RNA, operating on the RNA in a manner governed by the time history experienced by a reaction mixture that contains it, and reverse-transcribing the RNA into DNA.

In some embodiments, a piece of circular double-stranded DNA is composed of one or more blocks, with each block being composed of a “coding” region (which, when transcribed into RNA, provides instructions for the synthesis of proteins or provides a template for the formation of cDNA) that ends with a stop codon, and a “junk” region (which, when transcribed into RNA, forms a tail for the “coding region”) which encodes “Fourier modes” or other such basis functions, and ends with a terminator sequence (used to stop transcription). Strands of so-called “messenger RNA” formed by transcription of the DNA end up consisting of a “coding region” followed by a “tail”.

If the “tails” of the RNA are attacked from their ends by nuclease, and if the rate of removal of monomers (or units of monomers) is sensitive to both monomer (or unit) type and environmental conditions, then the time required for the “tail” of an RNA strand to be fully removed and the “coding” region of the strand to be “broken” (made unusable for protein synthesis) will be a function jointly of the tail sequence and the environment. The exact mechanism of this is articulated extensively in the other 2020 provisional patent applications made by the present author.

The short summary of the mechanism is that if one species of monomer (or block of monomers) may be identified as “binary one” and other species may be identified as “binary zero”, and if the rate of monomer removal is sensitive to a stimulus from the surrounding environment (e.g., the presence of a particular class of ion such as divalent metal ions, or temperature; see (Tanner et al., 2010; Bhan et al., 2019), and if that sensitivity also depends on the type of monomer being removed so that one may say that, for example, a higher rate of removal occurs when the monomer being removed is “binary one” and the stimulus is present, then, if the time series of the stimulus is represented as a binary number with “one” indicating the stimulus is present and “zero” indicating that it is not, then the rate of removal may be represented as the inner product (vector dot product where the binary number is expanded as a vector) of the binary representation of the strand sequence and the binary representation of the temporal evolution of the stimulus level, and the time-average rate of removal will be highest when this inner product is highest. This will mean, for example, that the translation of the mRNA into a protein or its reverse transcription into cDNA will be broken fastest under these conditions and more slowly under other environmental sequences. Depending on how the sensitivity is selected for the removal process, one may also set things up so that the average removal rate is slowest when the environmental conditions are met, etc. For much more detail, consult the prior 2020 filings by the same author.

In some embodiments, a circular or linear strand of double-stranded DNA comprises a number of blocks each having a “coding” region and a “junk” tail region that ends with a terminator. In the presence of the appropriate reagents and enzymes, the DNA transcribes into mRNA of the sort already described above, with a start region, a coding or symbol region, and a stop codon, then a tail. There are several different species of block, some which code for proteins in their coding section and some which code for cDNA in their coding section. There are also several species of “junk” tail.

The “coding regions” and the “junk tails” are paired up in such a way, and the frequency of the different species of block (combination of species of coding region and junk tail) are selected such that synthesis of different proteins is maximized under certain environmental time series and minimized or non-maximized under others. This occurs by the inner product leading to fastest removal of certain mRNA “tails” (thereby breaking operation of the mRNA in translation) under certain environmental time sequences, so the minimum production of that protein. Such a mechanism has already been described in brief and is described more extensively in other parts of this document and is described extensively in the prior filings of 2020 by the present author.

As an example, if the “coding regions” for two species of block are the same, but one has a “one cosine” tail and the other a “two sine” tail, then if the environmental signal is “one cosine” only or equal parts “one cosine” and “two sine”, the production of that protein will end more quickly than if the environmental sequence is “one sine” and has no “one cosine”, “two cosine”, or “two sine”.

In some embodiments, some of the blocks (“coding region” plus “tail”) will code in their “coding regions” for one protein (the “primary protein” or the “therapeutic”) while others will code for an antagonist (“blocker”) for that protein. And, in some embodiments, the tails will be chosen such that under certain environmental conditions the primary protein and its blocker are synthesized at the same rate so that the net “free” primary protein is negligible, but under different circumstances, the synthesis of the “blocker” ends before that of the primary protein, so that there is a net excess of unblocked primary protein. (And, in some, the reverse happens and there is excess blocker, but these cases can be considered under the same format if one simply renames the old blocker the new primary and the old primary the new blocker). In this fashion, the net effective dose of therapeutic generated in a unit of time by way of transcription and translation of the double-stranded DNA is sensitive to the time-evolution of the environment that the reaction mixture is part of or exposed to.

In some embodiments, in addition to blocks (note “block” here means “some DNA” not a “blocker” protein as in the paragraph above) used in translation (like those just described), or as an alternative to them, some of the blocks (“coding region” plus “tail”) will code in their “coding regions” for complementary DNA (cDNA) made by way of reverse transcription. Specifically, in some embodiments, the cDNA that is coded for in the “coding region” consists of short strands (oligos, e.g.) that represent one half of a sticky-ended double-stranded DNA segment. Different blocks code for different sticky-ended sequences. Some of the cDNA have the feature that part of them codes for one overhang (e.g., the 5′ overhang) and the region of bonding (when unmelted) of a sticky-ended piece. Some have the feature that part of them codes for an overhang that is complementary to the first (the 3′ overhang) and a region that is the complement in the bonding region for the double-stranded sticky-ended unit (see Tanner et al., 2010 for examples of sticky-ended DNA segments). In some embodiments, the sticky-ended overhangs are selected such that each is unique to the core region of the sticky-ended unit, so that if two or more sticky-ended segments come in contact, they form a longer strand that is a repeating of the motif encoded by a double-stranded single sticky-ended piece. In this way, the dose reverse-transcribed from the mRNA of cDNA associated with that particular motif controls that amount of “tails” of that particular motif in the product mixture. Some of the cDNA codes for proteins in the usual manner of the “coding region” of the mRNA or starting DNA, but also has an overhang that is associated with one of the unique tail types (but only on one end). So, it will attach to the tails and form up in DNA a replica of some mRNA bloc or, more properly, the original bloc of circular plasmid or linear DNA that coded for said block. The dose of “coding” cDNA formed through reverse transcription and “junk tail” cDNA formed through reverse transcription controls the amount of sticky-ended assembled cDNA blocks in the product mixture. In some embodiments, T4 DNA ligase, or an appropriate alternative, and some other appropriate reagents ligate the sticky-ended pieces and “coding” “roots” together to make double-stranded DNA (as T4 DNA ligase does the sticky-ended units in Tanner et al., 2010, for example) featuring coding regions and tails. Since the consumption of the mRNA tails controls the dose of synthesis via reverse transcription that occurs, just as it does translation in the earlier example just above, the temporal sequence of environmental conditions that occurs during the reaction ultimately regulates the dose of different species of DNA block synthesized via reverse transcription.

In some embodiments, the cDNA forms up sticky-ended units which are ligated with each other to form “Fourier-type” tails and “coding” roots (as just described), enzymes (e.g., T4 DNA ligase) then heal up the nicks and ligate everything into a DNA block, and then other enzymes insert the resultant blocks into a new plasmid or into the original plasmid from which the mRNA was transcribed. In some embodiments, a finished cDNA strand is double strand tail with a single-stranded “coding” overhang that gets its complement filled in with single nucleotides when incorporated into a plasmid or other piece of double-stranded DNA. In some other embodiments, the synthesized double-stranded DNA is blunt-ended and incorporated whole.

In some embodiments, the DNA synthesized by reverse transcription (with its dose level set by the evolution of the environment) get inserted back into the original dsDNA plasmid from which the mRNA was formed. In some, not only does this occur, but also random deletions from the plasmid occur. In some, the deletions are random nucleotide removals. In some, they are random excisions of whole “blocks” (“blocks” of the sort described above, formed via reverse transcription or originally present). In some, they are some other deletion process. In some embodiments, the time-average rate of insertion of new reverse transcription DNA and the time-average rate of removal of old “template for mRNA” DNA (a.k.a. past insertions) are equal to each other, so the time-average size of the plasmid does not change, over time, but its composition in terms of DNA blocks does. In some of these embodiments, some of the DNA codes for proteins and some codes for the cDNA that is ultimately inserted back into the plasmid as changes to it. Since the plasmid changes over time (Note: although the term plasmid is used throughout this discussion, it should not be considered restrictive. It should be interpreted to also signify linear DNA and, in fact, other kinds of information-encoding polymers or sequences of units), the “phenotype expression” (what protein translations occur) also changes over time. The “phenotype expression” may be sensitive to environment by the mechanisms articulated by earlier paragraphs, or it may not. Either way, the sensitivity of the reverse transcription to environment causes the genome in the plasmid and the “phenotype expressed” to change over time in response to the environment. That is, the genome and phenotype change in response to environment; the above described-machinery functions as a type of chemical recorder.

In some embodiments, a genome evolved by way of response to environment is inserted into or passed on to an offspring. In these embodiment, a form of synthetic Lamarckian evolution can be said to occur.

In some embodiments (in the preferred embodiment), the concentration of a particular species of ion (or one or more particular species of ion) serves as the environmental stimulus which regulates the digestion of nucleic acid “tails” (or other “Fourier-type” polymer strand or sequence of units, or other information-storing polymer strand or sequence of units).

In some embodiments, a plasmid or linear strand of DNA mutates in such a fashion by the mechanisms articulated above (those above involving “Fourier coding”, mRNA, etc.) that its instantaneous composition is a function of location along the path. That is, the stored genotype and/or expressed phenotype varies in a predictable way with distance along some path. In some embodiments, the reaction environment evolves because a particle or slug containing the reaction mixture traverses a spatial path at some speed such that the time-evolution of the traversed path is reflective of the path-wise evolution of the local environments along the path, and, by the mechanism described above, the DNA is therefore continuously edited, by way of one or more of the environmentally sensitive mechanisms articulated throughout this documents and in prior filings, during path transit, such that the stored genotype (DNA content) or expressed phenotype (e.g., proteins synthesized by translating and transcribing DNA) is a function of location along the transited path. That is to say, to formulation implements a TERCOM mechanism by way of “gene” expression and editing.

In some embodiments, reverse transcription into DNA that encodes a folding mock-protein RNA with a TERCOM tail and/or path-dependent conformation functionality is also provided. By way of example and not limitation, in some embodiments the presently disclosed subject matter includes retroviruses that place into an organism's DNA a sequence encoding a therapeutic RNA that itself has an active site that folds and a tail that when degraded by an exonuclease imparts the self-destruct-fuse type TERCOM-type functionality. Additionally, in some embodiments the DNA can encode a therapeutic RNA that features a path-dependent conformation.

As an example of a particular embodiment, one could use a folded RNA mock heparin with an appended protective tail, the tail of which has a sequence that is selected such that when an exonuclease, say RNAse, the removal rate for which is sensitive to both environment, in the form of local ion concentration, and removed nucleotide, attacks the tail the time-average rate at which the tail is consumed is greater for some environmental paths than for others and, therefore, the prospect of the active region of the RNA (the non-tail) surviving the transit time from the site of injection or absorption into the blood stream to the capillaries is greater for paths leading to capillaries in the desired target area (say, as an example, the vicinity of a stent of known location) and lesser for paths leading to capillaries in other areas, thereby achieving a time-fused self-destruction of portions of the injected dose that go off-course; all with this RNA/exonuclease reaction mixture enveloped inside of a liposome of the usual sort used in RNA vaccines or other therapies, but with pores added to the liposome in the form of, e.g., bacterial porins or other transmembrane porins, such that said pores allow the passage of information about said liposome's surrounding environment (such as instantaneous local ion concentration) to be passed to said reaction mixture. That and an experimental apparatus that can be used with said formulation to demonstrate this concept of TERCOM-type targeted delivery in a laboratory setting. In an alternative embodiment, the “active” RNA would be in an mRNA as found in RNA vaccines' perhaps an mRNA for defensin or another protein. In other embodiments, other approaches would be used, but all would share the idea of a polymer tail encoding information about a path in such a way that when operated on an enzyme or other reactant, a test is performed on the environmental path followed to see if it matches or does not match a preferred path. In even more distant alternative embodiments, no nuclease or other enzyme would be present, but the polymer carrying the map (be it RNA, protein, etc.) would instead interact with itself in a way that depends on the environmental path and the polymer sequence (the time evolution of the conformation of the particle with time from the moment of e.g. injection would depend on the spatial path taken).

In some embodiments, a DNA plasmid contains not only the DNA sequences to implement the above described “DNA which self-edits in response to environmental experience” (the above described DNA that makes mRNA which is environmentally regulated by attack of its “Fourier tails” and which is also reverse transcribed into cDNA that can be assembled and incorporated into the original plasmid or other DNA), but it also contains the DNA sequences (“genes”) necessary for the synthesis of all of the various enzymes used in the above-described reactions (reverse transcriptase, nucleases, polymerases, etc.), so that the transcription and translation processes also make all of the reactants required for operation of the device and implementation of such a system only requires insertion of this DNA into a plasmid within a cell. For example, into a competent cell supplied by New England Biolabs (hereinafter “NEB”). In some embodiments, the cell would edit its own DNA in response to environment and the changes would be heritable.

In some embodiments, the reagents for mutagenesis can be those of the Q5® Site-Directed Mutagenesis Kit from NEB. In some embodiments, the cDNA synthesized by the transcription and reverse transcription mechanisms (described herein and regulated by the environment) is inserted into a DNA plasmid using the Q5® Site-Directed Mutagenesis Kit. In some embodiments, the primers used are chosen appropriately. In some embodiments, the synthesized DNA is inserted into a plasmid using KLD Mix from NEB.

In some embodiments, the “sticky-ended” cDNA generated by the reverse transcription process is assembled by a ligating reaction mixture into long DNA blocks featuring coding for a “coding region” root and a “junk” “tail” (that is, for the “Fourier-tailed” mRNA) and used for subsequent insertion into a plasmid or into linear dsDNA. In some embodiments, that reaction mixture is the NEB Golden Gate Assembly Kit (BsaI-HF v2) offered by NEB. In some it is the NEB Golden Gate Assembly Kit (BsmBI-v2) from the same vendor. In some is it the Gibson Assembly Master Mix or Cloning Kit offered by the same vendor. In some it is one of the NEBuilder HiFi DNA Assembly Master Mix, the NEBuilder HiFi DNA Assembly Cloning Kit or, other such NEBuilder HiFi DNA Assembly kit formats. In some embodiments, it is the BioBrick Assembly Kit offered by the same vendor.

In some embodiments, PCR amplification is applied to the DNA generated by reverse transcription, prior to its incorporation into a DNA plasmid or linear DNA.

In some embodiments, where the DNA plasmid or linear DNA contains multiple blocks that function as templates for the types of mRNA (with “Fourier” “tails”) described in other parts of this document, a Cas9 type reaction mixture is used to enable insertion of DNA at desired sites. In some embodiments, ENGEN® Lba Cas12a (Cpf1) brand programmable DNA endonuclease from NEB is used. In some ENGEN® Spy Cas9 NLS brand programmable DNA endonuclease or ENGEN® Sau Cas9 brand programmable DNA endonuclease, both from NEB, is used. In some embodiments, the targeting RNA used by the Cas-type reaction mixtures is generated from a ssDNA template using the ENGEN® sgRNA Synthesis Kit, S. pyogenes brand single guide RNA (sgRNA) synthesis kit from NEB. In some embodiments, the ssDNA template is formed via reverse-transcription from the mRNA transcribed from the DNA in an environmentally sensitive fashion (see discussion in other parts of this document).

Thus, in some embodiments DNA is treated as the template polymer, mRNA as the synthesized polymer strands used in the removal process to make outcomes sensitive to environmental sequence, and proteins are treated as the therapeutics or their blockers. However, these terms are not meant to be restrictive and the descriptions should be understood to apply for polymers or chains of units in general, the nucleotides should be understood to mean also, more generally, monomers or units made of sequences of monomers (e.g., sticky-end DNA), and the therapeutics should be understood to be any of a variety of naturally occurring or synthesizable molecules or assemblies, not just proteins. The key aspect is that parts of a strand of information-containing material is copied and the processing or destruction of those parts occurs at a rate that is sensitive to environment while at the same time said copies are used to guide the synthesis of some therapeutic and its blocker or to achieve some other such desirable outcome.

The following describes some other embodiments, some of which have features that are different from those described above, but all of which share the feature that information sensed from the time evolution of a surrounding environment is coded into a polymer through a probabilistic process or used to affect synthesis of a polypeptide or other polymer; some share the feature that such coding is used to edit nucleic acids used to drive the synthesis of proteins. The other embodiments are as follows.

In some embodiments, the cDNA synthesized by reverse transcription has a tail, and this tail is removed by nuclease that does not attack the RNA. So, translation and reverse transcription are controlled separately.

In some embodiments, a combination of extension and tail removal are used to cause the DNA that results from the reverse transcription and mutagenesis process to depend in various ways on the environment. In some embodiments, “double doses of mRNA” are made, some portions of which are used in translation and some portions of which are used in reverse transcription to reformulate the DNA. If the environment does not consume the tails quickly, the reactions reincorporate into the DNA longer tailed reverse transcription blocks. In some embodiments, reverse transcriptions makes primers with coding regions and sticky-ended DNA (formed as two different cDNA that overlap to form sticky-ended elements) and the sticky-ended elements tail onto the back of the primer. In this way, you can grow a particular type of Fourier sequence “tail” onto the back of the primer in a way that is environmentally sensitive.

In some embodiments where a protein is synthesized by translation of a strand of nucleic acid, a tail is consumed and the nuclease attacks the stop codon. With the loss of the stop codon, the translation is disrupted (in some embodiments, the rate is greatly reduced), effectively stopping the translation relative to the rate at which it occurs for other species of strand in the mixture (e.g., which have different tail “junk” “motifs” and, so, slow down or stop at different times). In the manner already described, by selecting the mixture of species of strand, the dose of transcription product generated can be made sensitive to environmental sequence.

In some embodiments, a plurality of blocks are present in DNA. In some embodiments, each species of “coding region” has one unique species of “junk” tail associated with it. In some, each “coding region” has a one or more species of “junk tail” associated with it.

In some embodiments, RNA which is transcribed from DNA has after the protein coding region a stop codon and, after that, a long tail that encodes a “Fourier” mode. The tail is attacked from its end by a nuclease at a rate governed by the tail sequence and the environment. Meanwhile, translation of the “coding region” proceeds. Eventually, the tail is consumed and the nuclease attacks the stop codon. With the loss of the stop codon, the translation is disrupted, and its rate is greatly reduced, effectively stopping the translation relative the rate at which it occurs for other species of strand in the mixture (which have different tail “junk” “motifs” and, so, slow down or stop at different times). In the manner already described, by selecting the mixture of species of strand, the dose of transcription product generated can be made sensitive to environmental sequence.

In a variation of the immediately above embodiment, the nuclease attacks the RNA from the end bearing a promoter sequence or start codon rather than from the end bearing a stop codon; a “junk” tail which encodes a “Fourier mode” precedes the promoter and/or start codon. When the nuclease eats through the tail and reaches the promoter or start codon, it consumes these and translation is stopped or slowed (since there is no promoter or start codon). In the manner already described, by selecting the mixture of species of strand (by, for example, selecting the mix of species of segments in the DNA which guides the formation of the RNA; or, by selecting the mix of species of linear DNA supplied to the reaction), the dose of transcription product generated can be made sensitive to environmental sequence.

In some embodiments, a plurality of types of coding region are present in the DNA, rather than a single type. In some embodiments, the coding regions are separated from each other by “junk” DNA regions of various length, rather than by “junk” DNA regions of fixed length. In some embodiments, the lengths of the strands vary. In some embodiments, the lengths of the regions of “coding” DNA vary. In some embodiments, the lengths of the regions of “junk” DNA vary. In some embodiments, the compositions of the regions of “junk” DNA vary. In some embodiments, the compositions of the regions of “coding” DNA vary.

More particularly, the prior filings describe some embodiments where linear or circular segments of double-stranded DNA feature regions of “coding” DNA separated from regions of “junk” DNA, the former of which code for proteins and the latter of which contain single or repeating “junk” “motifs” that encode the basis functions of a Fourier decomposition or some other such set of basis functions (in the manner already described above and below). Translation factors (enzymes and co-reagents) synthesize strands of RNA using the instructions in the DNA. Transcription factors synthesize polypeptides using the instructions in the RNA. Nucleases attack the RNA, degrading it from one end while translation, reverse transcription and mutagenesis, or both occur, (or prior to their occurring), the rate of attack depending on the environmental sequence and the sequence of the “junk”, in the manner already described in the earlier filings and expanded upon below. As a consequence, the doses of unconsumed “coding RNA” or proteins left by transcription and translation are sensitive to environmental path.

In some embodiments, suppose each strand of some RNA transcribed from the DNA polymer features a number of identical “coding” regions interspersed at equal distances throughout the strand and separated by “junk” regions that feature repeating “motifs” of noncoding nucleotide sequence. Suppose further that half the strands have “junk” regions with a repeating nucleotide motif “[one] cosine” and half have “junk” regions with a repeating “junk” nucleotide motif “two cosine”. Now, suppose that monomer removal is sensitive to a stimulus in the same way described earlier, where removal is faster for one type of monomer (binary one) in the presence of the stimulus, but not for the other type of monomer (binary two). As explained in the paragraph just above, if the signal is a repeating “two cosine”, then “two cosine” RNA strands will be consumed more quickly, on average, than “[one] cosine” strands. As a result, the coding regions of the “two cosine” species of strand will be disabled (as the tail is exhausted and the enzyme starts to attack the coding the region) more quickly than the “[one] cosine” species. If the signal is a repeating “[one] sine”, then “two cosine” and “[one] cosine” strands will be consumed at the same average rate. In this case, the coding regions will be disabled at the same rate. As a result, the dose translated from the RNA will be greater in the presence of a “[one] sine” stimulus signal than in the presence of a “two cosine” stimulus signal, because the latter will lose more of its coding regions during a fixed reaction time whereas the former will retain more of them. This is assuming identical strand lengths. So, by this mechanism, selection of the mix of strand species compositions is tantamount to specifying one or more environmental sequences for which dosing is selectively greater (or selectively lesser) than it is for others. Again, if those environmental sequences correspond to environments found in a sequence of stops along a spatial path traversed by the “charge”, then specification of a mixture will be equivalent to specifying one or more spatial paths for which dosing is selectively greater (or selectively lesser) than it is for others. In a variation of this, each RNA strand has a single coding region followed by a “junk” tail with repeating motifs that code for each species of RNA strand the “Fourier modes” or other such basis functions. The operation is similar to that already described for a similarly-featured DNA: the environment regulates through consumption of the tail how long translation of the coding region will occur before the coding region is no longer protected by the tail from being degraded. If one assume that transcription occurs at a fixed rate and that translation of each generated unit of transcription product occurs for a duration set by the combination of the environmental sequence and the “junk” nucleic acid sequence (“motif” and length), it is apparent by inspection and for the reasons already articulated in other examples that the dose is regulated by the environmental sequence (or spatial path, if the spatial path itself determines the environmental sequence).

In some embodiments, the mechanism described just above where the number of translation sites on a strand is reduced over time (the mRNA has multiple “coding” and “junk” regions because the DNA has these between the site which promotes transcription and the site which terminates it) is used to generate mRNA used in reverse-transcription, not translation, or not only translation.

In some specific embodiments of the various types (single root-tail strands, strands with interspersed junk, etc.) where the environment regulates the attack of transcribed RNA, the mixture of species present in reaction is controlled not by the relative presence of different strands (linear or plasmids) of DNA, but, rather, by the relative presence of different segments of DNA on the same strand. These segments are the equivalents of the individual species of strand articulated in the earlier examples and each generates through transcription an independent strand of RNA (so, there is a mix of RNA strands that corresponds to the mix of DNA segments). However all of the specification of the “mix” occurs in the relative frequencies of the segments in the single strand of DNA.

In some embodiments, a liposome with a bilayer composed of appropriate lipids and populated with trans-membrane proteins such as bacterial wall porins that support nonselective ion diffusion, encapsulates a “charge” of aqueous solution that contains: (1) a circular DNA plasmid; (2) a reagent mixture that includes a cell-free protein synthesis composition (which includes amino acids, nucleotides, tRNA, transcription enzymes, translation enzymes, an energy source such as ATP or NAD, if needed, and other such factors; in the specific preferred embodiment, PUREEXPRESS® or NEBEXPRESS® from NEB, or the comparable reaction mixture used in the “Fundamental Dogma” kit from vendor microPCR); (3) a reagent mixture that includes an RNA-degrading exonuclease that does not attack double-stranded DNA (in some embodiments, Exonuclease T, as offered by NEB; (4) appropriate buffers (in a specific embodiment, the buffers recommended by NEB for use with the reagent kits); and (5) other reagents as needed. The circular plasmid is composed of a number of segments, each of which has the structure: (i) “coding” region, (ii) a “junk” region, and (iii) a “terminator” that causes transcription into RNA to end when reached by the transcription enzyme. The first contains a sequence of DNA that includes a promoter site for transcription, a start codon, a sequence that codes for a polypeptide, an end codon. The second contains a sequence formed as a repeating “motif” of junk DNA that encodes a Fourier mode in the manner already described in other parts of this document (e.g., if nucleotides of one sort represent binary one and those of another represent binary zero, the sequence might be 11110000 to represent “[one] sine” and “11000011” to represent “[one] cosine, and 11001100 to represent “two sine” and 10011001 to represent “two cosine”, etc.; in a specific embodiment, the four aforementioned motifs are used and pyramidine nucleotides are used to represent binary zero in the RNA while purine nucleotides are used to represent binary one, this being done because the presence of pyrimidine reduces the activity level of Exonuclease T, according to literature offered by NEB). When the RNA is formed via transcription, it is this tail that will protect the “coding” portion of the RNA from attack by the RNase for a length of time governed by the inner product of the environmental signal with the tail motif sequence (see discussion elsewhere), causing the dose of expression to be regulated by the environmental path or sequence (again, see discussion elsewhere in this document and in other filings by the present author). As noted in the literature, a double CC nucleotide sequence effectively stops Exonuclease T, so this is relied upon to help to prevent attack of the tRNA needed for translation, but if attack of the tRNA does occur, this can be accounted for by supplying sufficient tRNA for some to be lost to the RNase while the RNA strands (“mRNA”) are acted upon by the RNase during the environmental sequence (that is, while the “inner product” is performed). In a specific embodiment (which is the preferred embodiment for this document), the “coding region” codes for the protein expressed in the “Central Dogma” kit offered by vendor microPCR (the level of production of which can be measured using kits offered by that vendor); in some embodiments, other polypeptides are coded for. In a specific embodiment (which is the preferred embodiment for this filing), the “coding region” also includes the other components suggested by NEB in the instructions (as of the date of this filing) for its cell-free protein synthesis kits (e.g., PUREexpress) for inclusion in a region of template nucleic acid polymer (e.g., template DNA) to be transcribed; among these include a promoter region, a start codon, the code for the polypeptide, and stop codon a ‘T7 terminator’ or other such terminator that encourages the termination of transcription; in some embodiments, other or additional features are included. In a specific embodiment, only “[one] cosine” and “two cosine” are present in the composition, so the composition will be selective for (more dose) “[one] sine” signals relative to “[one] cosine” signals.

In the above embodiment, the concept of operation is that there is a differential rate of removal from the “junk” tail of purines and pyrimidines by Exonuclease T (RNase T) and that enzyme is also sensitive to the presence of certain factors, specifically divalent cations [for reference, see Wikipedia entry for “Exonuclease T” ] such as the Mg ions provided in, e.g., NEB′ “NEB 4” buffer (a.k.a. “Cutsmart”). It is presumed that just as the cobalt ion levels can affect relative reaction rates for TdT, that the relative rates of removal by Exonuclease T of different nucleotides (e.g., pyrimidines and purines) are sensitive to changes in the divalent ion concentration (e.g., sensitive to Mg ions, or Co ions, or Ca ions, or Zn ions, etc.; with that sensitivity discoverable through a literature review or experiment). The remaining operating concept is as already articulated throughout this document (the “inner product” and “Fourier mode” discussions).

Also, as mentioned in passing earlier, Exonuclease T requires a divalent cation, as noted by Deutscher, M. P. and C. W. Maxler in an article titled “Purification and characterization of Escherichia coli RNase T” in Journal of Biological Chemistry. Those investigators

observed that optimal activity is achieved at 2-5 mM Mg2+ or 1 mM Mn2+ and that Co2+ could also partially satisfy the need for divalent cations. They observed that Cu, Zn, Ba, Ca, Cd, and Hg ions showed no activity and that purified RNase T is inhibited by increasing ionic strength, with 50 pct inhibition at 100 mM KCl and essentially complete inhibition at 250 mM Kcl. They also found that pH was optimal at 8-9, with 50 pct activity at pH 7.2 and 9.6. Temperature also has an effect.

This portion of the text describes the features of some other embodiments of the same concepts outlined above.

In some embodiments, a demonstration kit (referred to also as an “educational kit”) to demonstrate bionano TERCOM is disclosed, and that kit consists of the apparatus described herein, plus a particle-encapsulated or unencapsulated formulation of one of the types described just above or elsewhere in this document, which is passed through said experimental apparatus during an educational experiment.

In some embodiments where RNA hosts the “Fourier modes”, the translation operation is performed as a final step, after the experience of the environmental path. The transcription process generates the RNA during or prior to the environmental path. The trimming of the “junk” tails proceeds during the environmental path, which runs for a fixed time. Depending on the sequence experienced, some species of RNA strands are fully consumed or have their “coding” regions damaged while others have some “junk” tail left and, therefore, their “coding” regions left. As a result, the relative composition of the RNA mixture by “junk” species will differ at the end (some “junk species will be missing or have suppressed presence) So, depending on environmental sequence, the total dose of polypeptide-coding RNA available to the subsequent translation stage will depend on environmental sequence. Or, in some embodiments, each species of tail has its own species of “coding” DNA associated with it on the strand, so the relative mixtures of polypeptides that results from transcription will vary with environmental path (as is intuitively clear by inspection from the discussions elsewhere in this document). If the “coding” species are a therapy-inhibitor pair (one inhibits or neutralizes the other), then that ratio will also govern the uninhibited or unsuppressed dose, as is again clear by inspection.

In some embodiments, some other nucleotide mapping is used to represent binary one and binary zero, and the particular choices are made by first performing an series of experiments on long strands of single monomer types to discover the removal rates under different conditions, as was done in Bhan et al., 2019 for testing the extension rates for different nucleotides. These experiments may be performed on various candidate enzymes to find the best mapping of the binary numbers to nucleotide and the best nuclease for use in this application.

In some embodiments, engineered nucleases and transcription and translation enzymes are used so that these can coexist while maintaining their activity. In some, tail digestion, transcription, and translation are staged.

In some embodiments, what would normally be the “charge” of a liposome is left unencapsulated as a slug of reaction mixture that is passed through an experimental apparatus like that outlined below.

In some embodiments, within a vesosome, the interstices of which are populated by the DNA and RNA reaction mixture of the sort described above (the one with RNA encoding the “Fourier” modes; in some specific embodiments, the specific one of these given earlier as an example embodiment), two or more species of thermally sensitive liposomes such as but not limited to those described in Tanner et al., 2010 (see also U.S. Pat. No. 7,769,423), or other sensitive liposomes or vehicles, are found, and these each contain a corresponding species of inhibitor of RNase activity, the inhibitive effects of which are different for different species of nucleotides between the different species of inhibitor. The mixture ratio of the two species of inhibitor varies depending on the environment sensed by the liposomes (as described in Tanner et al., 2010 for the release of different species of monomer), so the relative removal rates for the different species of nucleotide also varies with changes in environment. In this way is accomplished the “rate of shortening of tail or strand is an inner product” effect already described in other parts of this document. The liposomes and inhibitors replace the sensitivity of the RNase to environment. In some such embodiments, the liposomes release an ion that affects the nucleotide removal process, rather than a protein-type inhibitor.

In some embodiments, DNA from which RNA is transcribed has after the coding region a terminator sequence, the existence of which improves the rate of transcription of the RNA, and after that, a long tail that encodes a “Fourier” mode. The tail is attacked from its end by a nuclease at a rate governed by the tail sequence and the environment. Meanwhile, transcription of the “coding region” proceeds. Eventually, the tail is consumed and the nuclease attacks the terminator sequence. With the loss of the terminator sequence, the transcription rate is greatly reduced, effectively stopping the transcription relative the rate at which it occurs for other species of strand in the mixture (which have different tail “junk” “motifs” and, so, slow down or stop at different times). In the manner already described, by selecting the mixture of species of strand, the dose of transcription product generated can be made sensitive to environmental sequence. Embodiments of this type feature the transcription process being regulated by the environment rather than the translation or reverse transcription process being so regulated.

In some embodiments, RNA which is transcribed from DNA has after the protein-coding region a stop codon and, after that, a long tail that encodes a “Fourier” mode. The tail is attacked from its end by a nuclease at a rate governed by the tail sequence and the environment. Meanwhile, translation of the “coding region” proceeds. Eventually, the tail is consumed and the nuclease attacks the stop codon. With the loss of the stop codon, the translation is disrupted rate is greatly reduced, effectively stopping the translation relative the rate at which it occurs for other species of strand in the mixture (which have different tail “junk” “motifs” and, so, slow down or stop at different times). In the manner already described, by selecting the mixture of species of strand, the dose of transcription product generated can be made sensitive to environmental sequence.

In a variation of the immediately above embodiment, the nuclease attacks the RNA from the end bearing a promoter sequence or start codon rather than from the end bearing a stop codon; a “junk” tail which encodes a “Fourier mode” precedes the promoter and/or start codon. When the nuclease eats through the tail and reaches the promoter or start codon, it consumes these and translation is stopped or slowed (since there is no promoter or start codon). In the manner already described, by selecting the mixture of species of strand (by, for example, selecting the mix of species of segments in the DNA which guides the formation of the RNA; or, by selecting the mix of species of linear DNA supplied to the reaction), the dose of transcription product generated can be made sensitive to environmental sequence.

In some embodiments, a plurality of types of coding region are present in the DNA, rather than a single type. In some embodiments, the coding regions are separated from each other by “junk” DNA regions of various length, rather than by “junk” DNA regions of fixed length. In some embodiments, the lengths of the strands vary. In some embodiments, the lengths of the regions of “coding” DNA vary. In some embodiments, the lengths of the regions of “junk” DNA vary. In some embodiments, the compositions of the regions of “junk” DNA vary. In some embodiments, the compositions of the regions of “coding” DNA vary.

In some embodiments, the liposome is composed of soy-based lipids; in some, it is composed of other types of lipids. In some embodiments, the liposome has a porosity sensitive to temperature. In some, it has a porosity sensitive to pH. In some it has a porosity sensitive to some other environmental variable, measure, or stimulus. In some, in lieu of a liposome, some other type of “hollow shell” vehicle is used. In some embodiments, the vehicle is a polymer shell; in some, it is a metal shell; in some, it is a ceramic shell; in some it is a hydrogel shell; in some, it is composed of some other material. In some embodiments, some other semipermeable shell is used. In some embodiments, the liposome is substituted for with a piece of sponge-like material, which need not be hollow, since its holds the “charge” as a sponge would. In some embodiments, a spherical ball of hydrogel is used as such a “sponge”. In some embodiments, a spherical ball of polymer is used. In some embodiments, some other sponge-like material is used. In some embodiments, some other substitute for the liposome is used, it providing the same function of retaining the “charge” in some way separate from the surroundings of said “charge”. In some embodiments, the vehicle is a sealed dialysis tube or bag, or a similar sealed vehicle composed of semipermeable membrane or semipermeable material.

In some embodiments, the transmembrane proteins are member of the transient receptor potential cation channel subfamily M family. In some embodiments, one or more TRPM4 polypeptides are employed. In some embodiments, one or more TRPM6, TRPM7, and/or TRPM8 polypeptides are used. Human TRPM family members are disclosed as Accession Nos. NP_060106.2 (TRPM4), NP_060132.3 (TRPM6), NP_060142.3 (TRPM7), and NP_076985.4 (TRPM8) of the GENBANK® biosequence database. In some embodiments, they are some other type of transmembrane protein. In some embodiments, they are porins. In some, embodiments, a mixture of different transmembrane proteins and other such functional units is interspersed throughout the lipid bilayer or other such shell, forming a “fluid mosaic” bilayer, etc. In some embodiments, some other type of particle or constituent serves as one or more pores that offer one or more diffusion pathways for ions or for some other signaling agent such as some solute (e.g., salt, sugar, proteins, etc.). In some, the “pore” or “valve” of the liposome (i.e., the functionality of the liposome that permits exchange between the interior and exterior of the liposome; see U.S. Patent Application Publication No. 2009/0275031 for an explanation of “valve”) is a protein or other constituent that is sensitive to light, sound, heat, mechanical stimulus, or some other such stimulus.

In some embodiments, the “vehicle” is a sealed piece of semipermeable membrane with “pinholes” (these may be punched, drilled, laser-drilled, chemically etched, etc.) larger than the cutoff diameter of the semipermeable material made throughout it. In some embodiments, these pinholes are filled with a glue or filler that dissolves over time or in the presence of some stimulus, so that the effective cutoff diameter of the vehicle shell suddenly increases after some time or exposure to some stimulus. In some embodiments, this may be used to make a vehicle that allows, for example, ions to pass through the membrane continuously while synthesized polymers are retained within it until such a time as a stimulus occurs or enough time has passed, at which point the synthesized polymers are released through the newly open “pinholes”.

In some embodiments, the nucleases act only on DNA (they attack DNA that is being transcribed into RNA, not the RNA). In some embodiments, the nucleases act only on RNA that has been transcribed from DNA (they do not attack the DNA, only the RNA transcribed from it). In some embodiments, the nucleases act on both DNA that is transcribed into RNA and said RNA that is transcribed from said DNA (they attack both the DNA and the RNA). In some embodiments, neither DNA nor RNA have monomers removed; instead, a protein translated from the DNA/RNA has its monomers removed. In some embodiments, some other sets of chemical reactions which can be articulated as transcription and translation are affected in some manner by the environment, with that manner having the same type of environmental or spatial sensitivity described above.

Herein, frequent mention is made of DNA. In some embodiments, RNA or another polymer is used. The descriptions of operation for these (e.g., the descriptions of how the inner products are realized) should be understood to be fundamentally the same as the examples given for DNA, just with a different polymer, and the use of the specific example of DNA should not be interpreted to mean that the concepts as described are only for DNA. Instead, they should be considered to be inclusive of all polymers or other strand-like assemblies where the concepts of operation can apply.

In some embodiments, the output of the “charge” (the “therapeutic agent”) is RNA. In some, it is a polypeptide or protein complex. In some, it is cDNA generated via reverse transcription. In some, it is some other polymer; in some, it is some other reaction product.

In some embodiments, including some that are variations of those where DNA hosts the “Fourier modes” and some where RNA does, there is present in the “charge” reverse transcription factors. These factors reverse transcribe the RNA output by the transcription process.

Since the nucleases and their “inner product” operation (see discussions elsewhere) cause the dosage of transcribed RNA “coding regions to depend on the environmental path taken (the time series of some environmental signal) by the mechanism already articulated, the dosage (or composition) of transcribed RNA after (or during) partial digestion and transcription depends on the environmental path taken. If the reverse transcription factors create copies of the RNA in DNA (either in new, stand-alone DNA or by inserting the reverse transcriptions into the linear or circular strands used as the templates of the “charge” reaction via mutagenesis or similar; see earlier discussions), the reaction mixture can store a “memory” of the environmental sequence it experienced (see U.S. Patent Application Publication No. 2009/0275031 for a discussion of the concept of engineering “genetic memory”). In this way, as is apparent, the reaction mixture can remember the environmental path it has experienced. In some embodiments, mutagenesis factors are used to insert into the circular or linear DNA supplied as the reaction template nucleotide sequences that record the RNA and, therefore, the environmental history. In some embodiments, other factors selectively or arbitrarily remove segments of DNA from the circular or linear DNA while DNA generated by reverse transcription is added, so the size of the circular or linear DNA remains constant over time, but its composition evolves.

In some embodiments, the reverse transcription factors are introduced (either simultaneously with, or after, the partial digestion and transcription phases) to the “charge” (reaction mixture) of the detailed example from above that featured circular DNA as a template, RNA as the “Fourier” mode host for the “inner product”, and exonuclease T. In some embodiments, the reverse transcription factors are introduced (either simultaneously with, or after, the partial digestion and transcription phases) to the “charge” (reaction mixture) of the example above which used linear dsDNA and exonuclease V.

In a specific embodiment, PROTOSCRIPT® II brand reverse transcriptase (NEB) is introduced to the “charge” from the preferred embodiment for this text or the one for the 22 Jun. 2020 filing to USPTO by this author, and it generates cDNA in a dose (or relative composition of mixture) that depends on the environmental sequence experienced by the “charge” as a result of the RNA dose so depending.

In some embodiments, one of the site-directed mutagenesis kits offered by one or more vendors is used to insert into the DNA plasmids the record of the RNA that was synthesized and partially digested in a fashion that implemented the “inner product” discussed throughout this text or that was synthesized as a time-record of the environment as discussed in the earlier filings by this author and in the part of this text that discussed “sticky ends”.

The text that follows from this point is excerpted (in some cases, with minor edits) from the above-mentioned earlier filings. It is included here to aid the reader in interpreting such terms as “inner product” “Fourier-type” chemical recording and coding scheme, “Fourier mode” “junk tails”, “coding region”, “junk tails”, etc. It should not be considered to compete with the descriptions above of means of implementing a “chemical recorder” that regulates protein synthesis, DNA synthesis, or mutagenesis by environmentally regulating one or more steps in a process of transcription, translation, and reverse transcription.

In some embodiments, a formulation for the synthesis of a polymer is prepared which exhibits the feature that when passed through two or more different spatial pathways, each of which features a different sequence of local chemical environments, the formulation synthesizes polymers having different monomer sequences, with the sequences encoding information about the spatial environment which was traversed, and an apparatus is prepared which features two or more differing spatial pathways for said formulation to traverse, with the differing pathways featuring local chemical or physical environments that vary along each path differently.

In some embodiments, the formulation is encapsulated in a liposome or other vehicle that features pores or passive diffusion channels (or similar such features), which allow those ions that are sensed by the above-described formulation to pass into the liposome, where they can affect the recording reaction. In some variations of such embodiments, the pores or passive diffusion channels allow only small ions such as one or more of those of Ca, Co, Mg, Zn, Na, etc. to pass freely, but do not allow nucleotides or other recording units (e.g., sticky-ended DNA segments) to pass; in others, not only can small ions such as those listed pass, but also nucleotides (or other recording units); in both sorts of variation, however, the enzymes used for the polymer synthesis (e.g., TdT and/or T4 DNA ligase) and the assembled polymer chains cannot pass.

In some embodiments, the TdT-based formulation is encapsulated in a liposome. In some embodiments, the chemical recording formulation is unencapsulated. In some embodiments, it is encapsulated by a vesosome.

In some embodiments, some other nonselective passive ion transport channel (or some other appropriate such mechanism) is substituted for TRPM4 in one or more of the specific formulations detailed herein; in some of these, TRPM8 is used in lieu of TRPM4. In some embodiments, porins are used].

In some of these embodiments, the chemical recording formulation works on the principle of removal, rather than addition, of monomers or units. In some embodiments, the chemical recording formulation creates in a polymer sequence (or similar sequence of units in some chain of units) a record of the Fourier series of an environmental signal (or some others such vector basis or set of basis functions) rather than the time series. In some embodiments, each monomer (or other type of unit) may be classed into one of two types so that the sequence of the polymer chain (or other chain of units) may be represented as a bit sequence or as a binary number. In some embodiments, a plurality of types of polymer chains (e.g., a plurality of single-strand DNA, RNA, DNA, polypeptides, or other polymers or chains of units) are present in a solution; in some of these, the sequences of each of the type are selected so as to constitute one of the basis functions of the Fourier series (e.g., sin(2pi*x), cos(2pi*x), sin(2*2pi*x), cos(2*2pi*x), etc.; as an example, the sequence with repeating DNA sequence pattern AAAATTTT might represent bit sequence pattern 11110000 which represents temporal function sin(2pi*u*t) or spatial function sin(2pi*x)) (or, in some other embodiments, some other such set of basis functions) and an enzyme or other agent which removes monomers from polymers is present, with the rate of removal accomplished by said enzyme being sensitive to one or more environmental variable and having a different level of sensitivity for different monomer types. For example, in some embodiments, the reaction rate for the removal process may be sensitive to the concentration of one or more ions in the reaction solution for the removal of one type of nucleotide (“species A”) but not for that of another type (“species B”) (in such embodiments, the reaction rate sensitivity to the environment acts as a generalized “valve” of the sort articulated in U.S. Patent Application Publication No. 2009/0275031); or, there may be some other such differential sensitivity of removal rate to environment that gives differing relative rates of removal for differing monomer species. Or, as another example, in some embodiments, the concentration of the plurality of strands which are being “eaten” by the enzyme may vary in response to an environmental variable so that the ratio of enzyme to strand varies with environment for each strand type; in some of these, it may be regulated by some type of general “valve” of the types outlined in U.S. Patent Application Publication No. 2009/0275031. Or, in some embodiments, the strands of each type may be kept together in vehicles unique to each type and access to the interior of these vehicles by the enzyme or other removal agent is regulated by some type of general “valve” of the sort articulated in U.S. Patent Application Publication No. 2009/0275031, so that, again, the ratio of removal enzyme or agent to strand varies for each strand type in response to environments. Regardless of the particular details of the type of “valve” implementation, the net effect will be that the amount by which each strand is shortened will take the form of an inner product of the time series (or spatial series/pathwise series) of the environmental variable with the time series (or spatial series/pathwise series) of the function represented by that particular strand. So, for example, is the signal is y(t) and the strand sequence is a binary representation of sin(2pi*x) and the nominal removal rate is u so that sin(2pi*x)=sin(2pi*u*t), or approximately so, then the length of strand removed will be proportional to the integral of the product of the perturbations of y(t) with sin(2pi*u*t); that is, z=C*Int y′(t) sin(2pi*u*t) dt where Int is the integral operation, dt is a time step, u is as already defined, y′(t)=y(t)−y_bar where y_bar is the time average of y, and C is some proportionality constant. If C is chosen so that z is a measure of the percentage of a strand removed, an if all strands start with the same length regardless of which basis function they encode, then Fn=1−zn will represent the Fourier series amplitude associated with the n-th Fourier basis function. By inspection, one can see that this result can be generalized for any set of basis functions and for cases where the starting strands do not all have known lengths but have lengths whose statistics are known as a function of the encoded basis functions. The value of Fn or zn above can be recovered in post processing by recovering the strands from the reaction solution, grouping them by their sequence pattern (e.g., whether they have repeating pattern e.g., 11110000 representing sin(2pi*x) or repeating repeating pattern 11001100 representing sin(2*2pi*x), etc.) and measuring their length using gel electrophoresis, column filtration, or any of the other commonly-used such techniques (for finding molecule length) of biotechnology and laboratory chemistry. The average length of each such group, when subtracted from the average starting length of strands with that same sequence pattern, will give an indication of the amplitude of the corresponding Fourier component of the chemically-recorded environmental variable. The time-series of the recorded environmental variable can then be reconstructed by reversing the Fourier transformation computationally.

In some specific embodiment of the chemical recording mechanism described immediately above (the Fourier-type chemical recording mechanism), an ensemble of DNA sequences are synthesized, which strands of a single class all having the same repeating pattern and approximately the same length and with the patterns selected such that they encode a binary sequence (by, for example, having A represent binary one and G, T, or C representing binary 0). In some of these embodiments, the DNA is synthesized using an organism (e.g., recombinant DNA), in some it is chemically synthesized. In some such specific embodiments, an enzyme (and the associated chemicals it requires, such as buffers, possible a source of energy for operation, etc.; all of which would typically come in a reaction kit) is introduced to the ensemble of DNA strands together with a fluctuating concentration of ion or other additive that affect the relative rates at which different types of nucleotides are removed from the chain by the enzyme (the analog of CoCl₂ for addition, except for removal). The environmental stimulus (i.e. the concentration of the regulating ion, or of temperature, etc.) is then fluctuated over time (locally or by way of traverse of the formulation through a spatially-varying environment) causing the relative removal rates to fluctuate and the recording mechanism described above to occur. The reaction is then stopped, the strands separated by length, and each length-group sequenced to give a correlation of sequence with length and, therefore and by inspection, the Fourier series amplitudes.

In some specific embodiments like the ones above, nucleotides are removed from strands by an enzyme whose removal rate is not selective for nucleotide type (it lacks that particular environmental sensitivity), but the removal process competes with an addition process that introduces environmental sensitivity. In some of these embodiments, the nucleotides added back are the ones removed, so if the extension process features selectivity that is environmentally sensitive, strand length will depend on both a strand's starting sequence (which Fourier basis function it represents, for example) and on the environment. As a result, ending strand length as a function of strand starting sequence (the yet-to-be-edited remainder) will provide some measure of the Fourier composition of the environmental signal, after appropriate post-processing of the data (e.g., model-based post-processing).

In some embodiments of a chemical recorder of the presently disclosed subject matter, the formulation is modified by having a variety of substrates strands of single-stranded DNA (ssDNA), with the sequence of each type in the variety coding a particular Fourier series basis function (or some other basis vector or basis function) and with their average lengths possibly being longer than those in the experiment described in EXAMPLE 1, by reducing the concentration of TdT (and its accompanying chemicals) relative to ssDNA substrates (in some embodiments, in some it is left unchanged), and by introducing an enzyme (and its partner reactants) that causes a removal reaction that competes with the extension reaction caused by the TdT. Since the relative concentrations of nucleotides in solution will depend on the types removed by the removal enzyme, the composition added back to the strands will depend on both the environmental signal (through the TdT) and the local values of the Fourier series basis functions.

In some embodiments, the Fourier-type chemical recording mechanism described above is used as a sophisticated trigger mechanism that is made sensitive to spatial path or unique temporal signals; in some, it is used as a data recorder; in some, it is used as a data storage mechanism; in some, it is used in the context of targeted therapy; in some, it is used to implement in an organism an inherited or inheritable memory of the sort articulated in U.S. Patent Application Publication No. 2009/027503, Tanner et al., 2009, or Tanner et al., 2010; in some, it is used for other applications. In some embodiments, the Fourier-type chemical recording mechanism is used to implement terrain-contour-matching (TERCOM) (or digital scene area correlation (DSMC)) approaches to targeted drug delivery of the various sorts articulated in the

In some embodiments, interspersed through the strands in an ensemble are regions of DNA that code for the synthesis of proteins or for some other metabolic or metabolic-like operation. In some embodiments, each strand has multiple copies of the same encoding region interspersed through it. In some embodiments, all strands have the same (or nearly so) length, strands of each type have the same coding regions but the coding regions are different for different types of strand (different basis vectors), and all start with the same number of coding regions. In these various embodiments, even if the rate at which the coding regions are consumed is not affected by environment, the rate at which the in-between regions are consumed will be, so the number of coding regions present in the mixture for each “Fourier mode” (or other coded basis function) will depend on the particular sequence of environmental shifts experienced (that is, the level of expression of the coding regions will depend on the Fourier spectrum of the environmental signal or on the time-evolution of the environmental signal). In such a case, the number of copies of the coding region of the sorts found in each particular strand will reflect the amplitude (or e.g., “one minus it”; that is, the fewer the remaining copies, the stronger the signal was; etc.) of the corresponding Fourier mode in the environmental signal.

This concept can be generalized to a variety of signal expression approaches. For example, if florescent tags of different colors are attached to short segments of single-stranded DNA or RNA that complement the “encoding regions”, then these little tags will interact with the coding regions (stick to them); if the tags in free solution are then removed and the sample illuminated, the intensity of each the fluorescent tag's color will indicate the strength (or weakness) of the corresponding Fourier mode in the environmental signal.

In some embodiments, the same bits of “protein coding sequence” are found in all of the strands (despite their representing different basis function in their non-coding (or junk, the parts where the sin(2pi*x) etc. are encoded) regions, but they are found in different numbers for different basis functions in such a way that the scalar level of expression of the “coding sequence” still indicates the lengths of the various strand types and, so, the Fourier spectrum.

For example, if there are four types of strands, they may occur in concentrations of relative frequencies of 1, 10, 100, 1000 for sin(2pi*x), cos(2pi*x), sin=(2*2pi*x), cos(2*2pi*x); then, they level of the expression is a 4-digit base-ten scalar that can be converted back into the amplitudes associated with each of the individual basis functions by inspecting the value at each place in the number. For example, a signal level of two-thousand and twenty-three (2023) might be [structured] to correspond to 2 cos+0 sin+2 cos 2+3 sin 2.

In some embodiments of the presently disclosed subject matter as described above for Fourier chemical recording using removal, the starting strands are DNA; in some RNA; in some, single-stranded DNA; in some, polypeptides; in some, polysaccharides; in some, some other long chain polymer; in some, strings of magnetic beads; in some, a digital stream; in some, some other chain of units.

In some embodiments of the various sorts described above, the recorded or detected environmental variables are one or more of: temperature, ion concentration, Mg ion concentration, Ca ion concentration, Co ion Concentration, Zn ion concentration, K ion concentration, Na ion concentration, Cl ion concentration, pH, mechanical stress, light level, light frequency, sound level, pressure, salinity, sugar level, oxygen level, carbon dioxide level, ammonia level, hormone level, neurotransmitter level, cold, flavor, scent, angle, gravity, acceleration, radio-frequency energy level, ultraviolet light level, infrared light level, neutral particles arrival rate or dosage, collision energy, touch, pain, charge, reactivity, stiffness or modulus, yield strength, fracture toughness, audible sound level, infrasound level, ultrasound level, altitude, relative altitude, GPS signal strength, cell phone signal strength, some other such environmental variable, or any other such environmental variable.

In some embodiments, some other nonselective passive ion transport channel (or some other appropriate such mechanism) is substituted for TRPM4 in one or more of the specific formulations detailed herein; in some of these, TRPM8 is used in lieu of TRPM4.

In some embodiments, a different chemical recording formulation that that described in the particular embodiments is used. In some embodiments, the chemical recording formulation is unencapsulated; in some, it is encapsulated. In some, it is encapsulated by a vesosome such as is described in U.S. Patent Application Publication No. 2009/0275031, which is incorporated by reference in its entirety. In some embodiments, the chemical recording formulation is that described in Tanner et al., 2010.

In some embodiments, a chemical recording formulation different from those described in Tanner et al., 2010 or Bahn et al., 2019 is used. In some of these embodiments, the chemical recording formulation works on the principle of removal, rather than addition, of monomers or units. In some embodiments, the chemical recording formulation creates in a polymer sequence (or similar sequence of units in some chain of units) a record of the Fourier series of an environmental signal (or some others such vector basis or set of basis functions) rather than the time series. In some embodiments, each monomer (or other type of unit) may be classed into one of two types so that the sequence of the polymer chain (or other chain of units) may be represented as a bit sequence or as a binary number; for a further articulation of this “bit sequence” representation as described herein. In some embodiments, a plurality of types of polymer chains (e.g., a plurality of single-strand DNA, RNA, DNA, polypeptides, or other polymers or chains of units) are present in a solution; in some of these, the sequences of each of the type are selected so as to constitute one of the basis functions of the Fourier series (e.g., sin(2pi*x), cos(2pi*x), sin(2*2pi*x), cos(2*2pi*x), etc.; as an example, the sequence with repeating DNA sequence pattern AAAATTTT might represent bit sequence pattern 11110000 which represents temporal function sin(2pi*u*t) or spatial function sin(2pi*x) (or, in some other embodiments, some other such set of basis functions) and an enzyme or other agent which removes monomers from polymers is present, with the rate of removal accomplished by said enzyme being sensitive to one or more environmental variable and having a different level of sensitivity for different monomer types. For example, in some embodiments, the reaction rate for the removal process may be sensitive to the concentration of one or more ions in the reaction solution for the removal of one type of nucleotide (“species A”) but not for that of another type (“species B”) (in such embodiments, the reaction rate sensitivity to the environment acts as a generalized “valve” of the sort articulated in U.S. Patent Application Publication No. 2009/0275031, as occurs in Bhan et al., 2019); or, there may be some other such differential sensitivity of removal rate to environment that gives differing relative rates of removal for differing monomer species. Or, as another example, in some embodiments, the concentration of the plurality of strands which are being “eaten” by the enzyme may vary in response to an environmental variable so that the ratio of enzyme to strand varies with environment for each strand type; in some of these, it may be regulated by some type of general “valve” of the types outlined in U.S. Patent Application Publication No. 2009/0275031. Or, in some embodiments, the strands of each type may be kept together in vehicles unique to each type and access to the interior of these vehicles by the enzyme or other removal agent is regulated by some type of general “valve” of the sort articulated in U.S. Patent Application Publication No. 2009/0275031, so that, again, the ratio of removal enzyme or agent to strand varies for each strand type in response to environments. Regardless of the particular details of the type of “valve” implementation, the net effect will be that the amount by which each strand is shortened will take the form of an inner product of the time series (or spatial series/pathwise series) of the environmental variable with the time series (or spatial series/pathwise series) of the function represented by that particular strand. So, for example, is the signal is y(t) and the strand sequence is a binary representation of sin(2pi*x) and the nominal removal rate is u so that sin(2pi*x)=sin(2pi*u*t), or approximately so, then the length of strand removed will be proportional to the integral of the product of the perturbations of y(t) with sin(2pi*u*t); that is, z=C*Int y′(t) sin(2pi*u*t) dt where Int is the integral operation, dt is a time step, u is as already defined, y′(t)=y(t)−y_bar where y_bar is the time average of y, and C is some proportionality constant. If C is chosen so that z is a measure of the percentage of a strand removed, an if all strands start with the same length regardless of which basis function they encode, then Fn=1−zn will represent the Fourier series amplitude associated with the n-th Fourier basis function. By inspection, one can see that this result can be generalized for any set of basis functions and for cases where the starting strands do not all have known lengths but have lengths whose statistics are known as a function of the encoded basis functions. The value of Fn or zn above can be recovered in post processing by recovering the strands from the reaction solution, grouping them by their sequence pattern (e.g., whether they have repeating pattern e.g., 11110000 representing sin(2pi*x) or repeating pattern 11001100 representing sin(2*2pi*x), etc.) and measuring their length using gel electrophoresis, column filtration, or any of the other commonly used such techniques (for finding molecule length) of biotechnology and laboratory chemistry. The average length of each such group, when subtracted from the average starting length of strands with that same sequence pattern, will give an indication of the amplitude of the corresponding Fourier component of the chemically recorded environmental variable. The time-series of the recorded environmental variable can then be reconstructed by reversing the Fourier transformation computationally.

In some embodiments of the chemical recording mechanism described herein (e.g., the Fourier-type chemical recording mechanism), an ensemble of DNA sequences are synthesized, which strands of a single class all having the same repeating pattern and approximately the same length and with the patterns selected such that they encode a binary sequence (by, for example, having A represent binary one and G, T, or C representing binary 0). In some of these embodiments, the DNA is synthesized using an organism (e.g., recombinant DNA), in some it is chemically synthesized. In some such specific embodiments, an enzyme (and the associated chemicals it requires, such as buffers, possible a source of energy for operation, etc.; all of which would typically come in a reaction kit) is introduced to the ensemble of DNA strands together with a fluctuating concentration of ion or other additive that affect the relative rates at which different types of nucleotides are removed from the chain by the enzyme (the analog of CoCl₂ for addition from Bhan et al., 2019, except for removal). The environmental stimulus (e.g., the concentration of the regulating ion, or of temperature, etc.) is then fluctuated over time (locally or by way of traverse of the formulation through a spatially-varying environment) causing the relative removal rates to fluctuate and the recording mechanism described above to occur. The reaction is then stopped, the strands separated by length, and each length-group sequenced to give a correlation of sequence with length and, therefore and by inspection, the Fourier series amplitudes.

In some embodiments of the various embodiments described above, the monomer-removal enzyme used is a nuclease, which removes nucleotides.

In some embodiments of the sorts described above for “Fourier chemical recording” using “removal”, the starting strands are DNA; in some RNA; in some, single-strand DNA; in some, polypeptides; in some, polysaccharides; in some, some other long-chain polymer; in some, strings of magnetic beads; in some, a digital stream; in some, some other chain of units.

In some embodiments of the various sorts described above, the recorded or detected environmental variables are one or more of: temperature, ion concentration, Mg ion concentration, Ca ion concentration, Co ion Concentration, Zn ion concentration, K ion concentration, Na ion concentration, Cl ion concentration, pH, mechanical stress, light level, light frequency, sound level, pressure, salinity, sugar level, oxygen level, carbon dioxide level, ammonia level, hormone level, neurotransmitter level, cold, flavor, scent, angle, gravity, acceleration, radio-frequency energy level, ultraviolet light level, infrared light level, neutral particles arrival rate or dosage, collision energy, touch, pain, charge, reactivity, stiffness or modulus, yield strength, fracture toughness, audible sound level, infrasound level, ultrasound level, altitude, relative altitude, GPS signal strength, cell phone signal strength, some other such environmental variable, or any other such environmental variable.

Described herein is also an experimental setup and operating concept for an experimental apparatus that implements a demonstration of a chemical recording formulation being used to record the spatial path traveled by a slug of material by recording the environmental sequence experienced. In some embodiments, that description called for the slug containing the reaction mixture to be preceded and followed by water used to prime the line and to serve as the pusher. In some alternatives to those embodiments, the slug may be preceded and followed by a stream of oil, so as to prevent the solutes in the slug from leaving the slug via diffusion, a concept employed in certain microfluidics devices. In some embodiments of the apparatus, the streams that precede or follow the slug are composed of mineral oil, particularly, which serves to prime the line and as a pusher. The operation of this embodiment is as already described in the prior filing and below, except with the substitution of the mineral oil.

Second, sticky-ended DNA can be employed as a ‘monomer’ for incorporation into long-chains of DNA (the ‘polymer’) in an environmentally-sensitive DNA synthesis reaction like that described for TdT herein below.

In U.S. Patent Application Publication No. 2009/0275031, the notion of “valves” which regulate the process of probabilistic addition of monomers to a polymer was introduced. This is exemplified in an experiment using TdT as the enzyme that performed the addition of monomers (DNA nucleotides) to a polymer strand (ssDNA). In some embodiments disclosed herein, the ‘monomers’ are “sticky-ended” double-strand DNA segments with overhangs. T4 DNA ligase can be used as the enzyme which stitches these ‘monomers’ into a polymer, performing a role performed in Bahn et al., 2019 by the TdT for nucleotides. The regulation of the relative probabilities of adding one species of monomer versus another to the polymer strand was governed by controlling access to the nucleotides via a thermally-sensitive pores (variable-porosity liposomes were used as the “valve”). The regulation can also be accomplished using sensitivity of the reaction rate for monomer addition to a solute in the surroundings (Co ions). In some embodiments articulated herein, the reaction rate for the “sticky ended” monomers being appended by the T4 DNA ligase is similarly regulated by the concentration of a co-present solute. Specifically, T4 DNA ligase in both its wild and salt-accommodating forms have a level of activity for ligating “sticky end” DNA that is sensitive to salt concentration (i.e., the activity decreases when the salt level increases; see e.g., vendor notes from NEB). Also, the rate of activity is higher for properly matched sticky ends than for those that either have a missing nucleotide in the overlap region (e.g., a 2 nt overhang matching with a 1 nt overhang) or mismatched base pair sequences in the overlap region (a 2 unit overhang that has a not-fully-complementary sequence to the overhang it is intended to stick to). So, by introducing two species of sticky-ended DNA, a first species (call it “1”) with a “left” overhang that complements the “right” overhang, and a second species (call it “0”), with a “left overhang” that complements the right overhang of the first species but has a “right” overhang that is either shortened by one nucleotide of has a sequence that is only partially-complementary with the “left” overhang, it is possible one may set up a situation where the relative rates of addition of the overhangs differ and where this difference in relative rates can potentially be governed by the salt level. Or, if the maximum rate of ligation (rate of activity) is accomplished near the melting temperature of the overhang region, the sequences of the overhang regions may be engineered to cause the relative rates of addition to have differential sensitivity to temperature by, for example, causing one melt temperature to be above the nominal incubation temperature and the other to be below it so that perturbing the incubation temperature up accelerates the rate of ligation (the activity) for the former but slows it for the latter and a perturbation of the incubation temperature down does the reverse. Beyond these changes, the operations of the chemical recorder and spatially-sensitive polymer synthesizer work in the same way already articulated in the June 2020 filing by the present author (and below) and in the other filings and referenced sources.

Furthermore, in some embodiments, a sensitive particle is described and is considered an exemplary embodiment of the presently disclosed subject matter.

In some of these embodiments, the chemical recording formulation works on the principle of removal, rather than addition, of monomers or units, and, in some embodiments, is referred to as “the Fourier-type chemical recording mechanism”.

It should be noted that in the discussion below, reference is made to DNA, but it should be understood that the same concepts may be applied to RNA, single-stranded DNA, or to other polymers so long as the appropriate factors for monomer removal are present. Additionally, whereas reference is made to nuclease, this should be understood to include RNase of various types in addition to endo- and exo-DNases of various types.

Also provided herein are methods for implementing chemical recorders of the presently disclosed subject matter, wherein monomers are removed from a strand of nucleic acid or some other polymer in such a way that the removal process is sensitive to the environment in some fashion and differentially so depending on the monomer removed (monomer removal rate depends on the monomer type and the environmental condition, with the sensitivity to environment different for different monomers or ending sequences of monomers). In the prior text, a method of recording was described wherein sequences were preformed and encoded information in the form of predetermined “Fourier series” or other such basis functions. In one embodiment of this filing, a strand is made by a process of unit addition to a strand that follows the “noisy” or stochastic addition approach outlined in U.S. Patent Application Publication No. 2009/0275031 (see also Tanner et al., 2009; Tanner et al., 2010; Bhan et al., 2019). In some embodiments of this approach, blocks of monomers in the form of sticky-ended DNA or individual monomers in the form of nucleotides are added in such a manner that the local concentration of a particular species of unit or monomer in the strand reflects the level of the environmental variable at the time of reading. A strand formed in such a way is then used as the strand to be “eaten” by a DNase, the rate of monomer removal for which is a function of both monomer type (or local monomer composition at the removal site) at the removal site and local environmental conditions. As already described in the prior filings by the present author and elsewhere in this document, the overall average rate of consumption of the polymer chain (the rate at which it is “eaten”) may then be expressed as the “inner product” (vector dot product) of its local composition as a function along its length and the time history of the environment during the time of the consumption of the strand. Depending on how the sensitivities of the enzyme used to add monomers to the strand during its formation vary with environment and added unit (add monomer, e.g.) type, and depending on how the sensitivities of the enzyme used to later remove monomer units from the strands vary with environment and removed unit type (e.g., local composition of strand at removal site), the “inner product” (vector dot product) of the strand composition and the changing removal environment can be set up so that the rate of removal is: (1) maximized if the time-evolution of the environment at the time of addition matches the time-evolution of the environment at the time of removal, (2) minimized if the “addition environment” time-evolution matches the “removal environment” time-evolution, (3) is at an extreme (maximum or minimum) only when the “addition environment” time-evolution does not match the “removal environment” time-evolution, (4) etc. Such outcomes will also be affected by whether removal occurs from the same end that addition occurred at (LIFO; last-in-first-out) or whether removal occurs from the opposite end as addition (FIFO; first-in-first-out). Overall, the key observation is that by making the formation and consumption (“eating”) of a polymer chain sensitive to environment (including free monomer concentration or access to monomers) and local removal site composition, the average rate of consumption of the chain can be made such that it depends on how closely the time evolution of the environmental conditions at the time of removal match those that prevailed at the time of formation. This can then be used to govern transcription, translation, reverse transcription, and other such processes in the manners already described in this filing and other provisional patent applications made by the present author. For example, a “noisy” information encoding tail may be added to a protein-coding DNA sequence using TdT or sticky ends and T4 DNA ligase, and this may then be removed using environmentally-sensitive removal enzymes as described herein. The time until, e.g., translation of the coding region into protein “breaks” will be, e.g., minimized when the time evolution of the environment during tail removal matches that during tail addition.

Also disclosed herein are porins, particularly those derived from bacteria, as a way of making liposomes permeable to ions used in the regulation of the formation of an information encoding polymer. As would be understood, porins can also be employed to make liposomes and other vehicles “leaky” with respect to other components of the environment. See also U.S. Pat. Nos. 4,767,615; 4,921,644; and 8,753,673 as well as U.S. Patent Application Publication Nos. 2012/0190630, 2013/0289022, and 2019/0184030, each of which is incorporated by reference in its entirety for discussions of “leaky” liposomes

In some embodiments that implement such a recording mechanism, a plurality of linear double-stranded DNA strands are suspended in a solution (referred to here as “the charge”) that is encapsulated by a liposome composed of a single-bilayer membrane (a unilamellar liposome; in some embodiments, a liposome composed of soy-based lipids) which is populated by a number of transmembrane proteins (in some embodiments, a TRPM4 polypeptide) that function as nonselective ion channels that support facilitated diffusion (that is, they allow the passive diffusion of ions) of ions and other solutes. The DNA has the feature that is has two types of regions: “coding” regions, where the DNA sequences code for specific proteins, and “junk” regions, where the DNA has a non-coding sequence. In the preferred embodiment, all of the coding regions are identical (all code for the same protein). The coding regions on a single strand are separated from each other by regions of junk DNA of fixed length. The lengths of the strands are all identical or nearly identical, and the lengths of the coding regions are all identical, the lengths of the junk regions are all identical, the number of coding regions is identical or nearly so for each strand and the number of junk regions is identical or nearly so for each strand.

In some embodiments, besides the DNA, the “charge” also contains various buffers, solutes, and other reagents that assist with chemical reactions that occur within the “charges”. In some embodiments, the charges also contain: enzymes that remove nucleotides from DNA strands (e.g., endonucleases, exonucleases, RNase, etc.); enzymes that transcribe DNA into RNA; enzymes that translate DNA into proteins; cofactors for these enzymes; monomer units such as amino acids and nucleotides.

In some embodiments, the charge includes one or more of the following reagents, which are available from NEB (comparable offerings are also offered by other vendors such as Monarch): Nuclease P1, which is a single strand-specific endonuclease that is dependent on Zinc and works on both ssDNA and RNA (and, also dsDNA in some buffers); T5 exonuclease, which works on double-stranded DNA; exonuclease V, which attacks double-stranded DNA from both the 5′ and 3′ ends, requires ATP for the reaction, also requires Mg²⁺ for activity, and is inhibited by Ca²⁺; exonuclease V (RecBCD), which acts on both single-stranded and double-stranded DNA from both the 3′ and the 5′ ends; Exonuclease VII, which attacks single-stranded DNA from both the 5′ and 3′ ends, but is not active on linear or circular double-stranded DNA; Exonuclease T (also known as RNase T), which digests only RNA or ssDNA, from the 3′ end, has a different activity for RNA vs. DNA, may have difficulty digesting RNA molecules containing C residues at the 3′ terminus, and removes ssDNA extensions, but does not work as well when two cytosine nucleotides are in a row (with a single 3′ terminal C residue reportedly reducing action and a two-terminal residue reportedly essentially stopping it); Exonuclease I, which digests only ssDNA, from the 3′ end; Lambda exonuclease, which removes nucleotides from linear or nicked double-stranded DNA from the 5′ side; exonuclease III, which degrades both strands of linear or nicked double-stranded DNA from the 3′ side; DNase I, which cleaves DNA and acts on single-stranded DNA, double-stranded DNA, chromatin, and RNA:DNA hybrids; Mung Bean nuclease, which removes both 3′ and 5′ single-stranded extensions of double-stranded DNA without attacking the double-stranded DNA, and appears to have a requirement for Zn²⁺ (Zinc) but cannot be heat inactivated; Micrococcal Nuclease, which degrades single-stranded and double-stranded DNA and RNA; Nuclease BAL-31, which degrades DNA, RNA, and linear double-stranded DNA from both the 3′ and 5′ sides, and can also act as an endonuclease that cleaves at nicks, gaps, and single-stranded stretches, and can be heat inactivated in the presence of 30 mM EGTA.

In some embodiments, the “charge” includes one or more nucleases. In some embodiments, it includes all or part of one or more of any one of the cell-free reaction kits offered by NEB or miniPCR as described in the EXAMPLES.

The DNA strands within the “charge” come in a plurality of “species”, differentiated from each other by the sequences of their “junk” regions. The sequences of the “junk” regions are chosen to constitute a set of basis functions when considered in the manner described herein below. Specifically, suppose the monomer units (e.g., base pairs or nucleotides) may be grouped into two types: those that are removed more easily by the nuclease or nucleases that cohabit the “charge” when a stimulus (e.g., an ion concentration) is present and those that are not, with the former referred to as the binary one (“1”) or “A” species and the latter referred to as the binary zero (“0”) or “B” species. Then in some of the DNA strands, the junk regions may implement a “sine” pattern, which features a long stretch of binary one followed by a long stretch of binary zero (e.g., if the “junk” regions are 8 bp long, the sequence would be 11110000). In some, it would implement a “cosine” pattern, which features a short stretch of binary one followed by a long stretch of binary zero, followed by a short stretch of binary one (e.g., if the “junk” regions are 8 bp long, the sequence would be 11000011). In some, it would implement a “two sine” pattern, which features a short stretch of binary one, followed by a short stretch of binary zero, followed by a short stretch of binary one, followed by a short stretch of binary zero (e.g., if the “junk” regions are 8 bp long, the sequence would be 11001100). In some, it would implement a “two cosine” pattern, which features a very short stretch of binary one, followed by a short stretch of binary zero, followed by a short stretch of binary one, followed by a short stretch of binary zero, followed by a very short stretch of binary one (e.g., if the “junk” regions are 8 bp long, the sequence would be 10011001). And so on. Note that it may also be convenient to represent the binary sequences as vectors, so 10011001 is represented as [1,0,0,1,1,0,0,1]^{T}.

It should be apparent by inspection that if the stimulus which accelerates the removal of nucleotides from the DNA strands has some sort of temporal profile, the average rate of removal will vary across different species of junk region, depending on the species. Specifically, if the stimulus has some temporal profile and the sequence of the “junk” region has some sort of profile, then the perturbations to the average rate of removal will depend on some inner product of these. For example: if the level of a stimulus reactant or catalyst varies in the manner of a “two cosine” wave over a time-frame equal to the average time taken to remove 8 base pairs (averaged across all species of nucleotide), it might be represented as the binary sequence 10011001 or a vector form of that sequence [1,0,0,1,1,0,0,1]^{T}. Since the rate of monomer removal increases for “binary one” monomers if the stimulus is present (or at a high level), but not for “binary zero” monomers, the perturbation to the rate of removal experienced by the strand will be the inner product of the two “two cosines” (that is, of 10011001 and 10011001), which in this instance is 4. However, a “junk” region that featured a “[one]sine” sequence 11110000 would have an inner product of only 2, as would a “[one] cosine” (11000011) and a “two sine” (11001100). So, it is apparent that a strand featuring “two cosine” as its “junk” DNA “motif” will be consumed my the nucleases more quickly than one featuring “two sine”, “[one] cosine”, or “[one] sine”.

Since the “coding regions” are separated from each other within the strands (or from one or more of the ends of the strands) by the “junk DNA”, it is apparent that the number of instances of “coding” region present in the “charge” will decrease over time and that the time-average rate of this decrease will depend on the nature of the time variation of the stimulus signal in conjunction with the relative frequencies within the “charge” of strands hosting “junk” motifs of different species. So, for example, if a “charge” contains an even (50/50) mixture of “[one] cosine” and “two cosine” modified strands and the signal is a repeating “two cosine” signal, the perturbation to the rate of consumption of the strands caused by the signal will be an average of 2 and 4, or 3; if the signal is a repeating “one sine” signal, it will be an average of 2 and 2, or 2.

That is, the strands in aggregate will be consumed faster if the signal is a repeating “two cosine” than if it is a repeating “one sine”. If the “coding” regions are evenly dispersed throughout the strand (an example of such a strand would be one with three 8 bp “coding” regions evenly interspersed amongst four 8 basepair (bp) “junk” regions, so, a 56 bp vector: [junk,coding,junk,coding,junk,coding,junk]), and the “coding” regions are continuously transcribed or transcribed and translated throughout the time the chain is being consumed, and if this transcription and translation occur at a rate unaffected by the stimulus signal that governs the rate of monomer removal, then one sees by inspection that the total dose of transcription or translation will depend on whether the signal was a repeating “two cosine” or a repeating “one sine”. If the signal variation occurs because the environment experienced by the “charge” as the “particle” traverses a path varies with the path, then the total does of transcription or translation will depend on whether the spatial variation of the environment along the path was a “one cosine” or a “two cosine”. One can see by inspection that the operating principle described for the specific example will hold in the general case of any particularly specified mixture of strands bearing different “junk” species as their motifs. Specification of a mixture will be equivalent to specifying one or more environmental sequences for which dosing is selectively greater (or selectively lesser) than it is for others. If those environmental sequences correspond to environments found in a sequence of stops along a path through a flow network or along some other such spatial path, then specification of a mixture will be equivalent to specifying one or more spatial paths for which dosing is selectively greater (or selectively lesser) than it is for others.

As another example, in some embodiments each strand of polymer is a single coding region located at a protected or inert end (either way, end cannot be attacked by the monomer removing enzyme), with a tail of “junk” DNA appended to the other end (see discussion herein below). In some embodiments, half the strands have a tail with a repeating “junk” DNA motif “[one] cosine” and half have a tail with a repeating “junk” DNA motif “two cosine”. In some embodiments, monomer removal is sensitive to a stimulus in the same way just described above, where removal is faster for one type of monomer (binary one) in the presence of the stimulus, but not for the other type of monomer (binary two). As explained in the paragraph just above, if the signal is a repeating “two cosine”, then “two cosine” tails will be consumed more quickly, on average, than “[one] cosine” tails. As a result, the coding region of the “two cosine” species of strand will be disabled (as the tail is exhausted and the enzyme starts to attack coding the region) more quickly than the “[one] cosine” species. If the signal is a repeating “[one] sine”, then “two cosine” and “[one] cosine” tails will be consumed at the same average rate. In this case, the coding regions will be disabled at the same time. As a result, the dose transcribed or translated will be greater in the presence of a “[one] sine” stimulus signal than in the presence of a “two cosine” stimulus signal, because the latter will loss half of its coding regions part way through the reaction whereas the former will retain them both until the end. This is assuming identical tail lengths. So, by this mechanism, selection of the mix of tail species compositions is tantamount to specifying one or more environmental sequences for which dosing is selectively greater (or selectively lesser) than it is for others. Again, if those environmental sequences correspond to environments found in a sequence of stops along a spatial path traversed by the “charge”, then specification of a mixture will be equivalent to specifying one or more spatial paths for which dosing is selectively greater (or selectively lesser) than it is for others.

In some embodiments of such an example, the coding region is located at one end of the strand and has appended to it on one side, a long tail of the “junk” sequence. For example, the coding region may consist of a short region of double-stranded DNA that serves as the starting point for a transcription protein complex followed by a short region of single- or double-stranded DNA that codes for a protein or RNA sequence, followed by a long stretch of “junk” DNA featuring repetitions of some “junk” sequence such as “one cosine”. If in such an embodiment, the nuclease can only attack the strand from the side that features the “junk” DNA, the long stretch of “junk” DNA serves as a type of “fuse” that allows transcription of the coding region to occur until such a time as the nuclease finally “eats through” the “junk” DNA tail and reaches the coding region (where it attacks the coding region and breaks transcription). In some embodiments, therefore, the fuse can be a self-destruct fuse that causes self-destruction (e.g., by the sensitive nuclease) of the therapeutic payload (e.g., the active region of the mRNA) for portions of the dose that go “off-course” long enough (i.e., don't follow the path to the target site). In one specific such embodiment, the “charge” includes ssDNA with a double-stranded regions on its 5′ end that codes for the start site of an RNA transcriptase, followed by a “coding” stretch of ssDNA that codes for a specific protein, followed by a long ssDNA stretch featuring repeated instances of a single “species” of “junk” DNA.

In some embodiments, rather than nucleotides being removed from DNA in the manner outlined above, they are removed from RNA transcribed from the DNA. That is, the DNA contains the mix of “coding” and “junk” regions described above, both of which are transcribed into RNA (e.g., messenger RNA), but the nucleases act on the RNA, not directly on the DNA. The action is in the same manner already described above for nucleases acting on e.g., DNA. However, instead of the environment affecting yield by affecting an input to the transcription process (the DNA), it affects yield by affecting an input to the translation process (the RNA).

In some embodiments, linear or circular segments of double-stranded DNA feature regions of “coding” DNA separated from regions of “junk” DNA, the former of which code for proteins and the latter of which contain single or repeating “junk” “motifs” that encode the basis functions of a Fourier decomposition or some other such set of basis functions (in the manner already described above and below). Translation factors (enzymes and co-reagents) synthesize strands of RNA using the instructions in the DNA. Transcription factors synthesize polypeptides using the instructions in the RNA. Nucleases attack the RNA, degrading it from one or both ends while translation occurs, the rate of attack depending on the environmental sequence and the sequence of the “junk”, in the manner already described for DNA or polymers in general. As a consequence, the dose transcribed is sensitive to environmental path.

Specifically, in some embodiments each strand of some RNA transcribed from the DNA polymer features a number of identical “coding” regions interspersed at equal distances throughout the strand and separated by “junk” regions that feature repeating “motifs” of noncoding nucleotide sequence. In some embodiments, half the strands have “junk” regions with a repeating nucleotide motif “[one] cosine” and half have “junk” regions with a repeating “junk” nucleotide motif “two cosine”. In some embodiments, monomer removal is sensitive to a stimulus in the same way described earlier, where removal is faster for one type of monomer (binary one) in the presence of the stimulus, but not for the other type of monomer (binary two). As explained herein above, if the signal is a repeating “two cosine”, then “two cosine” RNA strands will be consumed more quickly, on average, than “[one] cosine” strands. As a result, the coding regions of the “two cosine” species of strand will be disabled (as the tail is exhausted and the enzyme starts to attack the coding the region) more quickly than the “[one] cosine” species. If the signal is a repeating “[one] sine”, then “two cosine” and “[one] cosine” strands will be consumed at the same average rate. In this case, the coding regions will be disabled at the same rate. As a result, the dose translated from the RNA will be greater in the presence of a “[one]sine” stimulus signal than in the presence of a “two cosine” stimulus signal, because the latter will lose more of its coding regions during a fixed reaction time whereas the former will retain more of them. This is assuming identical strand lengths. So, by this mechanism, selection of the mix of strand species compositions is tantamount to specifying one or more environmental sequences for which dosing is selectively greater (or selectively lesser) than it is for others. Again, if those environmental sequences correspond to environments found in a sequence of stops along a spatial path traversed by the “charge”, then specification of a mixture will be equivalent to specifying one or more spatial paths for which dosing is selectively greater (or selectively lesser) than it is for others. In a variation of this, each RNA strand has a single coding region followed by a “junk” tail with repeating motifs that code for each species of RNA strand the “Fourier modes” or other such basis functions. The operation is similar to that already described for a similarly featured DNA: the environment regulates through consumption of the tail how long translation of the coding region will occur before the coding region is no longer protected by the tail from being degraded. If one assume that transcription occurs at a fixed rate and that translation of each generated unit of transcription product occurs for a duration set by the combination of the environmental sequence and the “junk” nucleic acid sequence (“motif” and length), it is apparent by inspection and for the reasons already articulated in other examples that the dose is regulated by the environmental sequence (or spatial path, if the spatial path itself determines the environmental sequence).

In some embodiments of the type where the environment regulates the attack of transcribed RNA, the mixture of species present in reaction is controlled not be the relative presence of different strands (linear or plasmids) of DNA, but, rather, by the relative presence of different segments of DNA on the same strand. These segments are the equivalents of the individual species of strand articulated in the earlier examples and each generates through transcription an independent strand of RNA (so, there is a mix of RNA strands that corresponds to the mix of DNA segments). However all of the specification of the “mix” occurs in the relative frequencies of the segments in the single strand of DNA.

In some embodiments, a liposome with a bilayer composed of soy-based lipids and populated with TRPM4 trans-membrane proteins that support nonselective ion diffusion encapsulates a “charge” of aqueous solution that contains: (1) a circular DNA plasmid; (2) a reagent mixture that includes a cell-free protein synthesis composition (which includes amino acids, nucleotides, tRNA, transcription enzymes, translation enzymes, an energy source such as ATP or NAD, if needed, and other such factors; in the specific preferred embodiment, PUREEXPRESS® or NEBEXPRESS® from NEB, or the comparable reaction mixture used in the “Fundamental Dogma” kit from vendor microPCR); (3) a reagent mixture that includes an RNA-degrading exonuclease that does not attack double-stranded-DNA (in a specific embodiment, Exonuclease T, as offered by NEB; (4) appropriate buffers (in a specific embodiment, the buffers recommended by NEB for use with the reagent kits); and (5) other reagents as needed. The circular plasmid is composed of a number of segments, each of which has the structure: (i) “coding” region, (ii) a “junk” region, and (iii) a “terminator” that causes transcription into RNA to end when reached by the transcription enzyme. The first contains a sequence of DNA that includes a promoter site for transcription, a start codon, a sequence that codes for a polypeptide, an end codon. The second contains a sequence formed as a repeating “motif” of junk DNA that encodes a Fourier mode in the manner already described in other parts of this document (e.g., if nucleotides of one sort represent binary one and those of another represent binary zero, the sequence might be 11110000 to represent “[one] sine” and “11000011” to represent “[one] cosine, and 11001100 to represent “two sine” and 10011001 to represent “two cosine”, etc.; in a specific embodiment, the four aforementioned motifs are used and pyramidine nucleotides are used to represent binary zero in the RNA while purine nucleotides are used to represent binary one, this being done because the presence of pyrimidine reduces the activity level of Exonuclease T, according to literature offered by NEB). When the RNA is formed via transcription, it is this tail that will protect the “coding” portion of the RNA from attack by the RNase for a length of time governed by the inner product of the environmental signal with the tail motif sequence (see discussion elsewhere), causing the dose of expression to be regulated by the environmental path or sequence (again, see discussion elsewhere in this document and in other filings by the present author). As noted in the literature, a double CC nucleotide sequence effectively stops Exonuclease T, so this is relied upon to help to prevent attack of the tRNA needed for translation, but if attack of the tRNA does occur, this can be accounted for by supplying sufficient tRNA for some to be lost to the RNase while the RNA strands (“mRNA”) are acted upon by the RNase during the environmental sequence (that is, while the “inner product” is performed). In a specific embodiment (which is the preferred embodiment for this document), the “coding region” codes for the protein expressed in the “Fundamental Dogma” kit offered by vendor microPCR (the level of production of which can be measured using kits offered by that vendor); in some embodiments, other polypeptides are coded for. In a specific embodiment (which is the preferred embodiment for this filing), the “coding region” also includes the other components suggested by NEB in the instructions (as of the date of this filing) for its cell free protein synthesis kits (e.g., PUREEXPRESS®) for inclusion in a region of template nucleic acid polymer (e.g., template DNA) to be transcribed; among these include a promoter region, a start codon, the code for the polypeptide, and stop codon a ‘T7 terminator’ or other such terminator that encourages the termination of transcription; in some embodiments, other or additional features are included. In a specific embodiment, only “[one] cosine” and “two cosine” are present in the composition, so the composition will be selective for (more dose) “[one]sine” signals relative to “[one] cosine” signals.

In some embodiments, the concept of operation is that there is a differential rate of removal from the “junk” tail of purines and pyrimidines by Exonuclease T (RNase T) and that enzyme is also sensitive to the presence of certain factors, specifically divalent cations such as the Mg ions provided in, for example, NEW “NEB 4” buffer (a.k.a. “Cutsmart”). It is presumed that just as the cobalt ion levels can affect the relative reaction rates for TdT, that the relative rates of removal by Exonuclease T of different nucleotides (e.g., pyrimidines and purines) are sensitive to changes in the divalent ion concentration (e.g., sensitive to Mg ions, or Co ions, or Ca ions, or Zn ions, etc.; with that sensitivity discoverable through a literature review or experiment; see e.g., Bhan et al., 2019). The remaining operating concept is as already articulated throughout the present disclosure (the “inner product” and “Fourier mode” discussions).

Also, as mentioned in passing earlier, Exonuclease T requires a divalent cation. Optimal activity is achieved at 2-5 mM Mg²⁺ or 1 mM Mn²⁺ and that Co²⁺ could also partially satisfy the need for divalent cations. They observed that Cu, Zn, Ba, Ca, Cd, and Hg ions showed no activity and that purified RNase T is inhibited by increasing ionic strength, with 50% inhibition at 100 mM KCl and essentially complete inhibition at 250 mM KCl. pH was optimal at 8-9, with 50% activity at pH 7.2 and 9.6. Temperature also has an effect.

In some embodiments, rather than trimming the RNA, DNA is trimmed. As already noted, this might occur by trimming a ssDNA “junk” tail of “coding” dsDNA or by trimming from one or both ends dsDNA that has “coding” regions interspersed between “junk” regions that feature “Fourier mode” encoding. In some embodiments, there are multiple DNA strands of different “junk” species, and the environmental sequence determines how much of each strand is consumed thanks the “inner product” effect already describes. So, environmental sequence controls the dose expressed (as already described for these embodiments and others). This is true whether transcription and translation occur during the DNA shortening process (during the “inner product” operation) or after, as can be seen by inspection given the articulations already given above and below.

In some embodiments of such an alternative formulation, the “charge” within the liposome or other vehicle (or, e.g., within an unencapsulated slug) contains the following: (1) a mixture of linear double-sided DNA strands, each of one of a handful of different “species”, with “species” reflected in a repeated “junk” “motif” interspersed throughout the DNA and separating “coding” regions, identical on all of the DNA strands, that serve as templates for transcription (and subsequent translation) by a cell-free protein synthesis kit; (2) a reagent mixture that includes a cell-free protein synthesis composition (which includes amino acids, nucleotides, tRNA, transcription enzymes, translation enzymes, an energy source such as ATP or NAD, if needed, and other such factors; in the specific preferred embodiment, PUREEXPRESS® or NEBEXPRESS® from NEB, or the comparable reaction mixture used in the “Fundamental Dogma” kit from microPCR); (3) a reagent mixture that includes a dsDNA-degrading exonuclease (in a specific embodiment, Exonuclease V, which attacks double-stranded DNA from both the 5′ and 3′ ends, requires ATP for the reaction, also requires Mg²⁺ for activity, and is inhibited by Ca²⁺; in some embodiments, another nuclease can be used); (4) appropriate buffers (in some embodiments, the buffers recommended by NEB for use with the reagent kits); and (5) other reagents as needed.

As already described in discussion of the “Fourier” concept, the dsDNA in this embodiment is composed of regions that “code” for activity, and those that are “junk” that encodes “Fourier modes”. The “coding” regions are separated from the “junk” regions by a “terminator” that causes transcription into RNA to end when reached by the transcription enzyme (this assure the “junk” is not transcribed), and consists of a sequence of DNA that includes a promoter site for transcription, a start codon, a sequence that codes for a polypeptide, an end codon, and then the terminator. The “junk” regions contains a sequence formed as a repeating “motif” of junk DNA that encodes a Fourier mode in the manner already described in other parts of this document (e.g., if nucleotides of one sort represent binary one and those of another represent binary zero, the sequence might be 11110000 to represent “[one] sine” and “11000011” to represent “[one] cosine, and 11001100 to represent “two sine” and 10011001 to represent “two cosine”, etc.; in a specific embodiment, the four aforementioned motifs are used and pyramidine nucleotides are used to represent binary zero in the RNA while purine nucleotides are used to represent binary one, this being done on the presumption that the rate of degradation by the nuclease is affected by both the environmental signal (e.g., the level of some specific ion; in a specific embodiment, Ca²⁺) and by the species of nucleotides being removed, so that an “inner product” of the fashion already described occurs. In a specific embodiment, three coding regions are separated from each other and from the ends by four 24 bp “junk” regions that repeat 8 bp motifs. In a specific embodiment, only “[one] cosine” and “two cosine” are present in the composition, so the composition will be selective for (more dose) “[one] sine” signals relative to “[one] cosine” signals. In some embodiments, the “coding” regions have the features that enable them to be used as templates for protein synthesis, (specifically, in some embodiments of this variation, they follow the pattern recommended by NEB for its cell free protein synthesis kits). In some embodiments, a therapeutic or detectable polypeptide is generated (specifically, in the preferred embodiment of this variation, the polypeptide generates is that generated by the microPCR “Fundamental Dogma” experiment kit, which is detectable and has an established base of protocol for its detection and dose measurement. The operation of this formulation is apparent by inspection and is of the type that has already been articulated in other parts of this text, and in the prior filing, and can be summarized as “the inner product between the signal and the Fourier modes present in the junk DNA regulates the dose generated by regulating the amount of coding region digested or disabled during a given time period”.

In some variations of the above, strand digestion (by the enzyme), transcription, and translation, occur simultaneously. In some embodiments they occur sequentially, and the buffers are modified as necessary between steps. In some embodiments, different species of the DNA contain different species of “coding” region, so the composition of the product changes with environmental sequence in addition to, or as an alternative to, the dose. In some embodiments, translation does not occur, and the “output” of the formulation (the does or composition of which is sensitive to the environmental sequence) is the RNA generated by transcription. In some embodiments, the DNA is a double-stranded “coding” region with a single-stranded “junk” tail, as described in other text in this document.

In some embodiments, an educational “kit” to demonstrate “bionano TERCOM” is realized, and that kit consists of the spatial-path experimental apparatus described herein, plus a particle-encapsulated or unencapsulated formulation of one of the types described just above or elsewhere in this document, which is passed through said experimental apparatus during an educational experiment.

In some embodiments where RNA hosts the “Fourier modes”, the translation operation is performed as a final step, after the experience of the environmental path. The transcription process generates the RNA during or prior to the environmental path. The trimming of the “junk” tails proceeds during the environmental path, which runs for a fixed time. Depending on the sequence experienced, some species of RNA strands are fully consumed or have their “coding” regions damaged while others have some “junk” tail left and, therefore, their “coding” regions left. As a result, the relative composition of the RNA mixture by “junk” species will differ at the end (some “junk species will be missing or have suppressed presence).

So, depending on environmental sequence, the total dose of polypeptide-coding RNA available to the subsequent translation stage will depend on environmental sequence. Or, in some embodiments, each species of tail has its own species of “coding” DNA associated with it on the strand, so the relative mixtures of polypeptides that results from transcription will vary with environmental path (as is intuitively clear by inspection from the discussions elsewhere in this document). If the “coding” species are a therapy-inhibitor pair (one inhibits or neutralizes the other), then that ratio will also govern the uninhibited or unsuppressed dose, as is again clear by inspection.

In some embodiments, some other nucleotide mapping is used to represent binary one and binary zero, and the particular choices are made by first performing an series of experiments on long strands of single monomer types to discover the removal rates under different conditions. These experiments may be performed on not only exonuclease T but on other candidate enzymes to find the best mapping of the binary numbers to nucleotide and the best nuclease for use in this application.

In some embodiments, it may be necessary to use engineered nucleases and transcription and translation enzymes so that these can coexist while maintaining their activity. Or, it may be necessary to stage partial digestion, transcription, and translation.

In some embodiments, the “charge” of the liposome, above, is left unencapsulated as a slug of reaction mixture that is passed through an experimental apparatus like that outlined below.

In some embodiments, within a vesosome, the interstices of which are populated by the DNA and RNA reaction mixture of the sort described above (the one with RNA encoding the “Fourier” modes; in some specific embodiments, the specific one of these given earlier as an example embodiment), two or more species of thermally-sensitive liposomes or vehicles, are found, and these each contain a corresponding species of inhibitor of RNase activity, the inhibitive effects of which are different for different species of nucleotides between the different species of inhibitor. The mixture ratio of the two species of inhibitor varies depending on the environment sensed by the liposomes, so the relative removal rates for the different species of nucleotide also varies with changes in environment. In this way is accomplished the “rate of shortening of tail or strand is an inner product” effect already described in other parts of this document. The liposomes and inhibitors replace the sensitivity of the RNase to environment. In some such embodiments, the liposomes release an ion that affects the nucleotide removal process, rather than a protein-type inhibitor.

In some specific embodiments, DNA from which RNA is transcribed has after the coding region a terminator sequence, the existence of which improves the rate of transcription of the RNA, and after that, a long tail that encodes a “Fourier” mode. The tail is attacked from its end by a nuclease at a rate governed by the tail sequence and the environment. Meanwhile, transcription of the “coding region” proceeds. Eventually, the tail is consumed and the nuclease attacks the terminator sequence. With the loss of the terminator sequence, the transcription rate is greatly reduced, effectively stopping the transcription relative the rate at which it occurs for other species of strand in the mixture (which have different tail “junk” “motifs” and, so, slow down or stop at different times). In the manner already described, by selecting the mixture of species of strand, the dose of transcription product generated can be made sensitive to environmental sequence.

In some specific embodiments, RNA which is transcribed from DNA has after the protein coding region a stop codon and, after that, a long tail that encodes a “Fourier” mode. The tail is attacked from its end by a nuclease at a rate governed by the tail sequence and the environment. Meanwhile, translation of the “coding region” proceeds. Eventually, the tail is consumed and the nuclease attacks the stop codon. With the loss of the stop codon, the translation is disrupted rate is greatly reduced, effectively stopping the translation relative the rate at which it occurs for other species of strand in the mixture (which have different tail “junk” “motifs” and, so, slow down or stop at different times). In the manner already described, by selecting the mixture of species of strand, the dose of transcription product generated can be made sensitive to environmental sequence.

In a variation of the immediately above embodiment, the nuclease attacks the RNA from the end bearing a promoter sequence or start codon rather than from the end bearing a stop codon; a “junk” tail which encodes a “Fourier mode” precedes the promoter and/or start codon. When the nuclease eats through the tail and reaches the promoter or start codon, it consumes these and translation is stopped or slowed (since there is no promoter or start codon). In the manner already described, by selecting the mixture of species of strand (by, for example, selecting the mix of species of segments in the DNA which guides the formation of the RNA; or, by selecting the mix of species of linear DNA supplied to the reaction), the dose of transcription product generated can be made sensitive to environmental sequence.

In some embodiments, a plurality of types of coding region are present in the DNA, rather than a single type. In some embodiments, the coding regions are separated from each other by “junk” DNA regions of various length, rather than by “junk” DNA regions of fixed length. In some embodiments, the lengths of the strands vary. In some embodiments, the lengths of the regions of “coding” DNA vary. In some embodiments, the lengths of the regions of “junk” DNA vary. In some embodiments, the compositions of the regions of “junk” DNA vary. In some embodiments, the compositions of the regions of “coding” DNA vary.

In some embodiments, the liposome is composed of soy-based lipids; in some, it is composed of other types of lipids. In some embodiments, the liposome has a porosity sensitive to temperature. In some, it has a porosity sensitive to pH. In some it has a porosity sensitive to some other environmental variable, measure, or stimulus. In some, in lieu of a liposome, some other type of “hollow shell” vehicle is used. In some embodiments, the vehicle is a polymer shell; in some, it is a metal shell; in some, it is a ceramic shell; in some it is a hydrogel shell; in some, it is composed of some other material. In some embodiments, some other semipermeable shell is used. In some embodiments, the liposome is substituted for with a piece of sponge-like material, which need not be hollow, since its holds the “charge” as a sponge would. In some embodiments, a spherical ball of hydrogel is used as such a “sponge”. In some, a spherical ball of polymer is used. In some, some other sponge-like material is used. In some embodiments, some other substitute for the liposome is used, it providing the same function of retaining the “charge” in some way separate from the surroundings of said “charge”. In some embodiments, the vehicle is a sealed dialysis tube or bag, or a similar sealed vehicle composed of semipermeable membrane or semipermeable material.

In some embodiments, the transmembrane proteins are TRPM4 polypeptides as described herein. In some, the transmembrane proteins are TRPM8 polypeptides. In some, another type of transmembrane protein is employed. In some, embodiments, a mixture of different transmembrane proteins and other such functional units is interspersed throughout the lipid bilayer or other such shell, forming a “fluid mosaic” bilayer, etc. In some embodiments, some other type of particle or constituent serves as one or more pores that offer one or more diffusion pathways for ions or for some other signaling agent such as some solute (e.g., salt, sugar, proteins, etc.). In some, the “pore” or “valve” (see U.S. Patent Application Publication No. 2009/0275031, incorporated herein by reference in its entirety) is a protein or other constituent that is sensitive to light, sound, heat, mechanical stimulus, or some other such stimulus.

In some embodiments, the “vehicle” is a sealed piece of semipermeable membrane with “pinholes” (these may be punched, drilled, laser-drilled, chemically etched, etc.) larger than the cut-off-diameter of the semipermeable material made throughout it. In some embodiments, these pinholes are filled with a glue or filler that dissolves over time or in the presence of some stimulus, so that the effective cut-off-diameter of the vehicle shell suddenly increases after some time or exposure to some stimulus. In some embodiments, this may be used to make a vehicle that allows, for example, ions to pass through the membrane continuously while synthesized polymers are retained within it until such a time as a stimulus occurs or enough time has passed, at which point the synthesized polymers are released through the newly-open “pinholes”.

In some embodiments, the nucleases act only on DNA (they attack DNA that is being transcribed into RNA, not the RNA). In some embodiments, the nucleases act only on RNA that has been transcribed from DNA (they do not attack the DNA, only the RNA transcribed from it). In some embodiments, the nucleases act on both DNA that is transcribed into RNA and said RNA that is transcribed from said DNA (thy attack both the DNA and the RNA). In some embodiments, neither DNA nor RNA have monomers removed; instead, a protein translated from the DNA/RNA has its monomers removed. In some embodiments, some other sets of chemical reactions which can be articulated as transcription and translation are affected in some manner by the environment, with that manner having the same type of environmental or spatial sensitivity described above.

Frequent mention is made herein of DNA. In some embodiments, RNA or another polymer is used. The descriptions of operation for these (e.g., the descriptions of how the inner products are realized) should be understood to be fundamentally the same as the examples given for DNA, just with a different polymer, and the use of the specific example of DNA should not be interpreted to mean that the concepts as described are only for DNA. Instead, they should be considered to be inclusive of all polymers or other strand-like assemblies where the concepts of operation can apply.

In some embodiments, the output of the “charge” (the “therapeutic agent”) is RNA. In some, it is a polypeptide or protein complex. In some, it is some other polymer; in some, it is some other reaction product.

In some embodiments, including some that are variations of those where DNA hosts the “Fourier modes” and some where RNA does, there is present in the “charge” reverse transcription factors. These factors reverse transcribe the RNA output by the transcription process. Since the nucleases and their “inner product” operation (see discussions elsewhere) cause the dosage of transcribed RNA “coding regions to depend on the environmental path taken (the time series of some environmental signal) by the mechanism already articulated, the dosage (or composition) of transcribed RNA after (or during) partial digestion and transcription depends on the environmental path taken. If the reverse transcription factors create copies of the RNA in DNA (either in new, stand-alone DNA or by inserting the reverse transcriptions into the linear or circular strands used as the templates of the “charge” reaction via mutagenesis or similar; see earlier discussions), the reaction mixture can store a “memory” of the environmental sequence it experienced (the concept of engineering “genetic memory” is discussed in U.S. Patent Application Publication No. 2009/0275031) In this way, as is apparent, the reaction mixture can remember the environmental path it has experienced. In some embodiments, mutagenesis factors are used to insert into the circular or linear DNA supplied as the reaction template nucleotide sequences that record the RNA and, therefore, the environmental history.

In some embodiments, the reverse transcription factors are introduced (either simultaneously with, or after, the partial digestion and transcription phases) to the “charge” (reaction mixture) of the detailed example from above that featured circular DNA as a template, RNA as the “Fourier” mode host for the “inner product”, and exonuclease T. In some embodiments, the reverse transcription factors are introduced (either simultaneously with, or after, the partial digestion and transcription phases) to the “charge” (reaction mixture) of the example above which used linear dsDNA and exonuclease V.

By way of example and not limitation, Protoscript II Reverse Transcriptase (NEB) is introduced to “charge” from embodiments disclosed herein, and it generated cDNA in a dose (or relative composition of mixture) that depends on the environmental sequence experienced by the “charge” as a result of the RNA dose so depending.

In some embodiments, one of the site-directed mutagenesis kits offered by one or more vendors is used to insert into the DNA plasmids the record of the RNA that was synthesized and partially digested in a fashion that implemented the “inner product” discussed throughout this text or that was synthesized as a time-record of the environment as discussed in the earlier filings by this author and in the part of this text that discussed “sticky ends”.

In some embodiments, the presently disclosed subject matter relates to the fabrication of environmentally-sensitive materials and particles useful for the purposes of targeted drug delivery, but is also applicable more generally to the design and programming of any tailored system to respond in an appropriate way to changes in environment. In such embodiments, a core idea of the presently disclosed subject matter is to assemble from lipid bilayers, polymers films, etc. a composite material composed of layers of materials, mixtures of material elements, nested or neighboring shells, etc. in such a manner that the assembly exhibits a unique response when subjected to a particular environment and does not exhibit that response when subjected to a different environment, the composition being selected via a mathematical methodology.

Physical Problem. In some embodiments of the presently disclosed subject matter, in any of its various embodiments, a material particle or assembly is generated which exhibits a measurable sensitivity to its environment or to the sequence of environments to which it is subjected. In some embodiments, the objective is to cause a particle to release a carried payload such as a drug when it is in a specific location within the circulatory system of an animal or plant; in a related application, it is to cause some change in conformation or other externally-sensible feature of the particle which has the effect of steering the particle on the basis of environment as it makes its way through the circulatory system.

Mathematical Problem. The specific mathematical problem of interest is for the case of a continuous-transition, discrete-state Markov chain of with a single chain of finite extent and no periodic states; this is ergodic with stable long-run probabilities (if the chain is very large or infinite, it can be divided into a set including the initial state within which there is a high probability or remaining over some arbitrary time horizon, and a set which there is a low probability of entering). Such a chain is representative of the movement of blood and the particles it carries through the circulatory system. At each juncture, there is some probability of taking one branch over the other, but the system is closed so that every juncture can eventually be visited after a long enough period of time. A particle or system can move through this network with the particle or system having some means of causing changes in its internal state (e.g., its porosity, whether a sensing mechanism is on, etc.) or external state (e.g., charge) when exposed to a specific sequence of environmental conditions.

In this case, the control problem is differentiated from traditional deterministic control in the face of random uncertainty by the fact that the actuator authority will never be sufficient to drive the system along a specific desired trajectory. Instead, the best that can be hoped for is to influence the transition probabilities with the goal of having the statistics of the system's trajectory be as close as possible to some desired ideal (e.g., maximize the frequency some specific desirable site is visited or minimize the time spent over some undesirable site).

Physical Compositions. A number of approaches familiar to those skilled in the state-of-the-art of chemistry, biology, and bottom-up and top-down nano- and micro-fabrication to physically generate particles which can be made to be responsive to the environmental experience.

In general, a composite tailored material can be modeled as a network of mass flow elements that is analogous to a network of electronic devices, with porous materials analogized as electrical resistors, contained volumes analogized as capacitors, stores of chemicals analogized as batteries, etc. This understanding of mass transport networks will be familiar to those skilled in the state of the art of mass transport. Like electrical networks, mass transport networks can be connected into circuits that encode certain computational functionalities, and the parameters of the elements of these networks can be selected to exhibit specific types of functionality, a process that is analogous to coding in hardware.

For the specific problem of interest, it is desirable to realize through a simple fabrication process a material that releases a dose of a drug when subjected to a specific environment or sequence of environmental conditions that would be representative of travel to a particular region of the body, but which does not release the drug when subjected to other sequences, which are representative of travel to other regions of the body.

In some embodiments, a particle is formed as a composite shell composed of alternate layers of material that is resistive to the flow of a solute and of a material that is stores the solute, with this shell enveloping a volume that is high is concentration of the drug. The first material represents a resistance, the second a capacitance, and the enclosed volume a battery or large charged capacitor. The enveloped volume might be a volume of liquid with a high concentration of drug contained within it, a soluble particle or droplet of drug, a particle of soluble or permeable solid impregnated with the drug, etc. The capacitive volume might be a thin film of water or some other solvent; a sponge-like layer capable of absorbing water or some other solvent, like that used for contact lenses; or some other material that can serve as a capacitive volume. The resistive material can be a lipid bilayer like those used to form liposomes, a porous polymer, a thin film of neighboring or overlapping solid chips, or some other such material.

By using different types of resistive material, each of which exhibits a more or less porosity, depending on the environment to which it is exposed, the composite shell can be made to function in such a way that it is highly likely to releases at a target location and is highly unlikely to release at other locations.

The operation of a particle of this form is as follows: when the particle is not in the region of interest, but is moving along a trajectory through the circulatory system that will lead it to do so, layers from the innermost to the outermost will become porous sequentially such that the capacitive layer between each is able to charge. So, for example, the first resistive layer will become porous when exposed to an environment like that along the first leg of travel to the target site, allowing the first capacitive layer to charge up to the concentration within the core volume. Then, when the next leg is reached, the next resistive layer will become porous, and the second capacitive layer will charge. This process continues until the particle is at the delivery site, at which point the outermost layer becomes porous, allowing the dose of drug stored within the various capacitive layers to discharge out of the particle and into the environment. After the particle leaves the target region, the layers become impermeable and the process starts again. If the particle takes a track that does not lead to the desired target area, the number and sequence of layer openings will not be correct and it will not be possible to achieve a release except in rare circumstances, these forming false positive events in a statistical sense.

A number of variants of this embodiment can be implemented, all varying slightly in their method of operation, but being substantially similar to that describe above. In some embodiments, the innermost layer is closed when the outermost is open, causing only the dose within the capacitive layers to be released; in another, all layers are open at the time of release so that a dose flows from the central volume to the surroundings. In some embodiments, when on the path leading to the target site, the layers open sequentially and remain open in all environments downstream of the one first opening, causing each capacitive layer to charge to the same concentration as the central source volume; in another, only one resistive layer is open at a time, causing the charge within the capacitive layers to cascade from one capacitive layer to the next, dropping by roughly half with each cascade; in others, layers open out of sequence but remain open so that when the target area is reached, the source is connected to the target environment through a high-porosity pathway; in others, some combination of these happens.

In some embodiments, the source volume of solute is located in a central volume; in others, it is one or more layers interleaved with the capacitive and resistive layers. In others embodiments, small spherical source volumes are each enveloped by one or more resistive layers and distributed throughout a capacitive volume that is itself bounded by resistive layers; in some of these the encapsulated source particles are contained within a larger spherical volume; in others, they are contained in a thin layer between two spherical shells or planar or cylindrical layers.

In some embodiments, the source of drug can be distributed throughout the various capacitive layers so that these charge individually and continuously, but only become interconnected when a specific environmental sequence is experienced. In this embodiment, which has the advantage of being easy to fabricate, capacitive layers only charge up substantially when they have had a long period of being sealed on each side by impermeable resistive layers; this occurs when they are on tracks that are not those leading to the target location. When the particle is on a track towards release, the capacitive layers communicate with each other and eventually release as a group.

In some embodiments, release does not depend on the sequence of environments being experienced, but only on a very specific environment being experienced. Some of these embodiments can involve a volume encapsulated by a single resistive film or multiple layers of film like those already described, but all of the same type. Others can involve a series of capacitive layers separated by resistive layers, all of the same type. In these embodiments, the resistive film is more porous in environments like those found in the vicinity of the target area and less porous in other environments. This differential of porosity leads to the drug or other solute being released preferentially at the target location.

The fabrication of all of these embodiment and others can be achieved by way of a number of different fabrication techniques. In some embodiments, lipid bilayers like those used in liposomes or synthetic biology are used; in others, polymer films or particles are used; in others, other techniques are used.

In some embodiments, fabrication is achieved by structuring the particle as a multi-layer liposome carrying a water-soluble drug, with each resistive layer formed by a variable-porosity lipid bilayer and each capacitive layer formed by a thin film of water. In some embodiments, such liposomes are formed by a process of lipid bilayer formation and extrusion to a given diameter, which correlates with number of layers. In some embodiments, if different formulations are used for each bilayer, the processes of encapsulation and extrusion can be staged in sequence to give a multi-layer liposome with the desired mix of layers. In some embodiments, also with different formulations for each layer, the different films are formed at once and their sequence of ordering from inner layer to outer layer is random for each individual liposome.

In some embodiments, a lipid bilayer serves as capacitive layer and a water film serves as the resistive layer; this variation is relevant when a drug is insoluble in water but can be dissolved in an oil or the lipid bilayer. In some embodiments, the lipid bilayers can form up as spherically-symmetric spherical shells; in others, as cylindrical rolls; in others, as sheets; in others as some other shape. Formation and sorting of these various shapes will be familiar to those skilled in the state of the art.

In some embodiments, the particle is formed from polymers by way of a top-down process. In some embodiments, a film of one type of polymer is deposited as a capacitive layer and a film of another type of polymer is deposited on top of this film as a resistive layer. The resulting multi-layer film is then rolled up as a cylindrical roll.

In some embodiments, film is rolled around a cylindrical core that is composed of or impregnated with the drug; in embodiments, the cylindrical core is a polymer impregnated with the drug or a metal coated with or impregnated with it. These embodiments are similar in nature to drug-eluting stents, which have a high-concentration of drug separated from the environment by a polymer through which the drug diffuses, except that in the embodiments described here, the resistance of the barrier material is a function of environment so that elution only occurs in the presence of a specific environment or sequence of environments.

In some embodiments, which form an alternate application to the preferred application described here, a drug-eluting stent or similar biomedical device is coated with a film made of a polymer or other suitable material, which film is sensitive to environment. The sensitivity to environment of the film is selected in such a way that the stent or other device only elutes when certain environmental conditions are present in the region of the blood stream where the stent is located. If other conditions exist, these film will be relatively more impermeable. In this way, the release of the stent can be shut off if the person in which it is placed is subject to some experience which would make the release of the drug dangerous. As a concrete example, if there would be a negative interaction with the drug and other drugs that can be in the blood stream, elution will be turned off if those other drugs are detected by the film. Or, as another concrete example, a stent that can elute a blood thinner does so when chemical or environmental indicators suggestive of an impending blockage are detected, but otherwise does not; or normally does so, but stops when indicators suggest that continued release would be dangerous.

In some embodiments, the capacitive layer itself is impregnated with particles of drug and the roll has no core or some inert core. As a specific example, a polymer with a high water content, such as those used in contact lenses, can serve as the capacitive layer and can be impregnated with particles that elute a drug into it. This polymer layer can be capped by resistive films of variable porosity which regulate release of the drug that accumulates in the capacitive layer from it into the environment. The two- or three-layer film can then be rolled up as a cylinder and cut into short segments giving short cylindrical particles as the drug-delivery particle.

In some embodiments, polymer films are formed by way of a process known as spin-coating. In this process, a solid substrate like a silicon wafer is spun and the polymer in an uncured form, or its precursor, is poured onto the spinning wafer, forming a thin film. The thin film is then cured into a solid or rubber-like elastic solid via some process like exposure to UV radiation, heat, chemicals, etc. A second layer is built upon the first in the same manner. In some embodiments, each one or more of the films has embedded within it chemical or particulate additives. In some embodiments, these are added in bulk before curing; in others, they are added by a process of patterning and impregnation after the formation of the film but before curing; and in still others they are added after curing. In some embodiments, after all of the layers are complete, the multi-layer sheet of film is cut into small pieces which are allowed to self-roll; in others, the sheet is rolled into a long thread-like cylinder and then cut into short pieces; in others, some other technique is used to form the sheet into individual particles.

These various fabrication techniques will be familiar to those skilled in the state of the art. The techniques for forming and rolling the spin-coated films have been demonstrated by researchers at the Massachusetts Institute of Technology in the context of fabricating polymer bands which change color under strain.

In the polymer-based embodiments described above, the form of the particle is a cylinder, but In some embodiments formed using similar materials or by way of similar processes, the shapes can take the form of spherical particles, sheets, etc., depending on the details of the processing technique used.

In some embodiments, which are variations on the former, a single film composed of a matrix and an additive is used, with one serving as the capacitive medium and the other serving as the resistive medium. The additive can be a powder of particles or fibers, or a connected mesh or truss-structure, or some other such material. The matrix can be a solid, a gel, or some other such material. Depending on the environment, the conformation of the particle can change is some desirable way. For example, in some embodiments, a particle composed of a mesh of environmentally-sensitive fibers can be embedded within a compliant polymer can contract when environmental conditions cause the mesh fibers to shorten, closing up the porous spaces between the nodes of the mesh and limiting diffusion out of the particle and also changing its size. In some embodiments, source particles can be distributed throughout a sponge-like matrix enveloped by a stiff film and environmentally-driven expansion or contraction of the matrix can change its resistance to movement of solute through it.

In some of the above embodiments and in some other embodiments, the films are composed of locally distinct regions, each with their own features such as level of sensitivity to various different environmental signatures. In some embodiments, the patterning is accomplished by a top-down process of masking the polymer, applying a paste of the dopant or submerging the masked film and substrate into a solution containing the dopant and allowing it to diffuse into the polymer. In another, different sources of dopant are printed onto the film via ink-jet printing, screen-printing, or some similar method that deposits droplets or coats of this material; the dopant is allowed to leach from the coating into the polymer; and the coating is then stripped. These various top-down approaches are similar in general concept to doping a solid film in, for example, semiconductor fabrication. In another class of embodiments, the film is itself composed of a number of subparticles that have been assembled and fused or bonded by way of some intermediate matrix or bonding material; this approach is sometimes seen in large-scale rubber mats that are from chopped and fused multi-colored rubber pieces. In yet another class of embodiments, the films are formed by depositing droplets of different liquid polymer precursors or mixes, allowing these droplets to spread up against each other as the liquid levels, and then curing the polymer. In still other embodiments, the film starts as a variety of beads that are mixed and spread and then pressed into a film using heat and pressure or some other such mechanism; at a larger scale, this approach is found on some arts and crafts techniques.

Regardless of the embodiment, the key feature is that the film is formed of neighboring regions that each have different responses to their environment so that the overall porosity of the film will be a scalar function of some vector of environmental variables or sequence of environmental variables.

In some embodiments, the presently disclosed methods relate to formulating and fabricating environmentally-sensitive drug particles which exhibit location-specific release within the circulatory system after the fashion of terrain contour matching. In some embodiments, the presently disclosed subject matter addresses targeted drug delivery using micro- or nano-fabricated particles that estimate of their own location within the body and release drugs near target locations selected on the basis of offline medical imaging.

Targeted delivery techniques commonly use chemical targeting via cell-borne receptors, genetics, or an ex vivo stimulus such as heat or radio waves that prompts spatially-localized release.

With respect to the presently disclosed subject matter, particles or formulations that estimate their own location within the body by correlating vectors of sensed environmental variables (e.g., temp., pressure, salinity, sugar levels, pH, etc.) against a carried map release their drug in the vicinity of a target site on the basis of this location estimate; this approach closely related to terrain contour matching (TERCOM), used in aircraft navigation. In some embodiments, top-down particle formulations are realized: thin sheets of permeable hydrophilic gel, clad with a quilt of polymer materials and charged with an eluting drug, are fabricated by spin-coating and dicing, then allowed to form swiss rolls; in another embodiments, multilaminar shells of environmentally-sensitive lipid bilayer, separated by thin films of water and enveloping droplets of drug in solution, are fabricated chemically. In both such embodiments, the barrier layers are quilted irregularly from a variety of semipermeable materials, each sensitive to a different stimulus, the path length for diffusion to the surroundings (the ‘Manhattan distance’) is a scalar function of the environmental vector in a manner approaching a perceptron.

In designing the detailed composition of such materials, the circulatory system is modeled. In some embodiments, the circulatory system is modeled as a parameterized, closed, lumped-element flow network of resistors, capacitors, and fins, driven by an actuator disc, and embedded within a field, itself modeled as a coarse unstructured mesh of heat-generating and reactive malleable solid, with which it locally exchanges heat and solutes, giving rise to predictable variations in the local circulatory environment. Its traverse is modeled as a discrete-state, continuous-transition Markov process with region of the body (mesh element) as Markov state. The Markov process is cast as a source of symbol sequences representing the path taken during a circuit through the circulatory network; the lumped-element model is cast as a transducer which maps place to environment.

This modeling approach requires the estimation of model parameters. In some embodiments, parameters of the model can be estimated from synthetic tissue analogs used for surgical device development, and particle design (specification of the pattern and composition of the particle layers) is cast as a problem of robust optimization of material parameters with a goal of balancing type I and II errors in release.

The particular sequence of environments that a particle experiences during a circuit of the circulatory system can depend on the context; that is, it can depend on whether the individual is sitting, standing, recline, etc. and on whether they are at high altitude, low altitude, in a warm environment, in a cool environment, mostly indoors, mostly outdoors, etc. If a particle or formulation is engineered only release under certain contexts (in addition to at a specific location), then the release will be targeted to location or context, or both.

By way of example and not limitation, the polymer coating of a drug eluting stent can be engineered to limit the rate of elution when certain environmental conditions are experienced by it (or when not). Because the stent does not move, the shifts in its environment will be related to shifts in context rather than location, so the environmental selectivity of the release mechanism will have the effect of being be selective for context rather than location.

In some embodiments, such as those where a particle is traversing the circulating system, the particle or formulation can be engineered to release only when in the desired location and under the desired context. Or, in some of these other embodiments, the context can be used to prevent release except under certain conditions.

Besides being useful for enhancing the effectiveness of a therapeutic, such a mechanism can allow the implementation of a digital rights management mechanism for engineered therapies, be they genetic or chemical. For example, in some embodiments, an engineered or tailored material is used to limit release so that is only occurs under authorized conditions; in others, an engineered organism or synthetic biological system can perform similarly.

In still other embodiments, release can be structured to occur only when both a certain context and a certain key are present, be that release specific to location as in targeted-release or fixed in the circulatory system as with a stent. In some such embodiments, the key can take to form of another chemical or marker being present; in some of these, a specific RNA strand can serve as key, for example. In all of these embodiments, and in other similar embodiments, the tailored material or mechanism can be said to be implementing drug delivery under a permissive action link.

### Conformation-Changing Peptides and Polymers.

In some embodiments, the presently disclosed subject matter relates to targeted drug delivery using synthesized peptides or other synthetic polymers or particles that evolve their conformation in response to the local environment found at their location within the body, or on the basis of their being exposed to a sequence of such environments, in such a manner that the release of a carried drug by the particle or the therapeutic activity or chemical activity of the particle is made a function of location within the body or circulatory system.

Targeted delivery techniques commonly use chemical targeting via cell-borne receptors, genetics, or an ex vivo stimulus such as heat or radio waves that prompts spatially-localized release. As set forth herein, in some embodiments particles or formulations release their drug in the vicinity of a target site by estimating their own location and releasing when in the target vicinity. These techniques are analogous to terrain contour matching (TERCOM) and digital scene matching area correlation (DSMAC), techniques used in aircraft navigation.

Here, a particle changes its conformation in response to environment, and the chemical activity or therapeutic activity can be made to be a function of the particle's location within the body. As with the approaches described in the referenced applications, the particle effectively estimates its location by way of correlation with measured environmental signatures and then links its activity to this estimate or to sequences of the same.

In some embodiments, a large molecule drug with this engineered sensitivity is designed to be inert when not is the physical vicinity of a target location and active when in that vicinity. In some embodiments, the particle serves as a ‘sabot’ for a particle of drug, releasing itself from the active particle when in the proper location. In this second instance, the delivery functionality is separated from the therapeutic functionality.

It is noted that in any of the embodiments of the compositions and methods of the presently disclosed subject matter, the particles, delivery vehicles, and/or formulations need not alter their compositions and/or conformations in response to variations in vivo environmental stimuli in a binary manner. Thus, when the present disclosure refers to a change in composition and/or conformation that results in one or more desired activities, that desired activity need not be entirely absent when the compositions and/or conformations are in the “inactive” form. Rather, contemplated within the scope of the presently disclosed subject matter are compositions and/or conformations in response to variations in vivo environmental stimuli that can be matters of degree. By way of example and not limitation, an “active” composition or conformation can be one that releases detectably more of a drug and/or that has some quantitatively or qualitative increased level of activity although the composition or conformation could have some detectable level of activity when in the “inactive” form provided that whatever difference exists between the active and inactive forms can be measured and/or has some biological and/or therapeutic relevance.

Different embodiments can make use of different combinations of environmental variables. In some embodiments, particles are sensitive to one or more of temperature, pressure, salinity, sugar levels, pH, etc., or are sensitive to one or more other environmental variables, or to combinations involving one more of all of these. The particles can be considered to be estimating their own location within the body by correlating vectors of these sensed environmental variables with carried map represented by the design parameters of the particle, making this approach similar to terrain contour matching (TERCOM) or other similar techniques such as digital scene matching area correlation (DSMAC).

In some embodiments, the particles are realized in a bottom-up fashion. In some embodiments, they are realized via chemistry or multistep combinations of chemistry and processing. In some embodiments, they are realized via a process of polymer synthesis. In some embodiments, the polymers are long chains. In some embodiments, they are realized via a process of peptide synthesis. In some embodiments, they are realized via a process of protein synthesis. In some embodiments, they are realized via synthesis of a protein complex. In some embodiments, they are realized via some other bottom-up chemical or nanofabrication process.

In some embodiments, the particles are realized in a top-down fashion. In some embodiments, they are realized by implementing chips using top-down micro- or nanofabrication processes; in some embodiments, these chips are biodegradable or inert; in some, they carry a releasable payload. In some embodiments, they are realized by top-down fabrication of meta-particles formed from long strands that fold upon each other in one or more ways, depending on environment, and, therefore, behave like a protein. In some embodiments, a hair-sized or smaller polymer strand is formed with regions doped or coated or otherwise treated in such a manner as to cause the strand to fold in a particular fashion. In some embodiments, the particle is a folded strand of memory material; in some, this memory material is a memory metal; in some, it is a memory metal coated with a polymer coating; in some, it elutes a drug in some folded conformations but not others, or its rate of elution is a function of its specific conformation; in some, it is a drug which elutes, and its rate of elution is a function of its specific conformation.

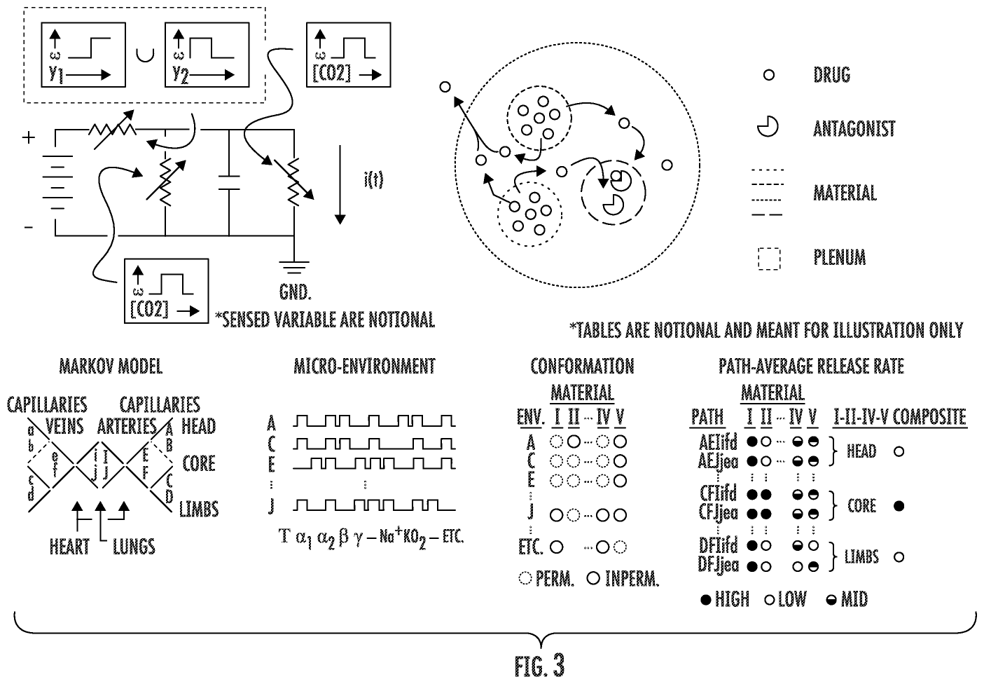

In some embodiments, the particles are designed by selecting the parameters of their design such that the particle conformation exhibits the desired behavior as a function of the environment of the circulatory system at different locations. In some embodiments, in designing the particles, the circulatory system is modeled. In some embodiments, the circulatory system is modeled as a parameterized, closed, lumped-element flow network of resistors, capacitors, and fins, driven by an actuator disc, and embedded within a field, itself modeled as a coarse unstructured mesh of heat-generating and reactive malleable solid, with which it locally exchanges heat and solutes, giving rise to predictable variations in the local circulatory environment. In some embodiments, the traverse of the body or circulatory system or other system is modeled as a discrete-state, continuous-transition Markov process with region of the body (mesh element) as Markov state. The Markov process is cast as a source of symbol sequences representing the path taken during a circuit through the circulatory network; the lumped-element model is cast as a transducer which maps place to environment. A schematic diagram of the presently disclosed subject matter is shown in FIG. 3.

This modeling approach requires the estimation of model parameters. In some embodiments, parameters of the model are estimated from synthetic tissue analogs used for surgical device development, and particle design (specification of the pattern and composition of the particle layers) is cast as a problem of robust optimization of material parameters with a goal of balancing type I and II errors in release or activity.

The particular sequence of environments that a particle experiences during a circuit of the circulatory system can depend on the context; that is, it can depend on whether the individual is sitting, standing, recline, etc. and on whether they are at high altitude, low altitude, in a warm environment, in a cool environment, mostly indoors, mostly outdoors, etc. If a particle or formulation is engineered to only release or become active under certain contexts (in addition to at a specific location), then the release will be targeted to context, or to both location and context.

In some embodiments, a drug in the form of a synthesized peptide can have its sequence of amino acids selected such that the potential energy well of the peptide, which varies with conformation, exhibits several local minimums, one of which can be a global minimum, each separated by a barrier with its own activation energy, measured from the base of the minimum to the peak of the area. Through proper amino acid sequence selection, the relative levels of these minima and the levels of the activation energies separating them can be made to be some function of the environment of the circulatory system, with such factors as temperature, salinity, oxygen level, lighting level, etc. playing a role. If properly selected, the barriers will change over time in such a way that convergence of the conformation to a particular state will be preferred under one environment and convergence to a different state will be preferred under a different environment, the rate of convergence depending in the heights of the energy barriers. For example, the sequence can be engineered to cause the particle to take on an inert conformation in certain environments where activity of the particle is not desired and to take on active conformations in environments where the particle would be therapeutic. In some embodiments, the progression of changes in the shape of the potential energy well can be structured such that a particle can achieve a specific desired configuration, such as a therapeutic one, only if a specific sequence of environments is experienced. In some embodiments, this is accomplished by structuring changes in the heights of the activation energy barriers such that a particle must move to a second conformation from a first before it can move to a third and this can only happen along a specific path. Along other paths, the barrier between the first and second and the first and third remained large so that transition to the third cannot occur even when the barrier between the second and third is lowered, the second never having been reached. In some embodiments, other, similar, such arrangements involving more than one local minimum are used.

In some embodiments, the changes in environment experienced by the particle result from changes in environment caused by changes in context rather than by those caused by location, or by changes caused by the combination of location and context. In these embodiments, the environment can change due to, for example, an increased rate of respiration, an increased metabolism, sleeping, or some other such change in context. In some embodiments, the particle is sensitive only to changes caused by changes in location of the particle within the body.

For example, in some embodiments, the conformation of a particle at some fixed location such as at a stent is engineered to cause it to be inert and attached when certain environmental conditions are experienced but active and free-floating when others are experienced. Because the particle does not move until active, the shifts in its environment will be related to shifts in context rather than location, so the environmental selectivity of the release mechanism will have the effect of being be selective for context rather than location.

In some embodiments, such as those where a particle is traversing the circulating system, the particle or formulation can be engineered to become therapeutically active or release only when in the desired location and under the desired context. Or, in some of these other embodiments, the context can be used to prevent release or activity except under certain conditions.

Besides being useful for enhancing the effectiveness of a therapeutic, such a mechanism can allow the implementation of a digital rights management mechanism for engineered therapies, be they genetic or chemical. For example, in some embodiments, a large molecule or some other tailored particle is designed for activity or release only under select conditions such that release or activity only occurs under authorized conditions. An engineered organism or synthetic biological system can perform similarly, either through genetic mechanisms or by incorporation of the aforementioned embodiments.

In still other embodiments, activity or release is structured to occur only when both a certain context and a certain key are present, be that activity or release specific to location as in targeted-release or fixed in the circulatory system as with a stent. In some such embodiments, the key can take to form of another chemical or marker being present; in some of these, a specific RNA strand can serve as key, for example; in others, a specific molecule such as an antibody can serve as key.

In embodiments just described, where activity or release is made sensitive to context, in whole or in part, and in other similar embodiments, the tailored molecule or mechanism can be said to be implementing drug activity or drug delivery under a permissive action link.

An advantage of this mechanism is that the drug or drug delivery mechanism can be tailored to activate or release in a location within the body circulatory system without requiring specific knowledge of the disease being addressed or the cells to which the therapy is being applied and also without using external targeting aids such as heat pads or radio waves.

Additionally, particles of the sort described can be used to introduce location-sensitivity to other problems in mass transfer and chemistry or can be used for more complex targeting problems such as context-sensitive drug targeting or release.

In some embodiments, the vehicles of the presently disclosed subject matter encompass RNA, and in particular, the vehicles can function as RNA vaccines and/or cancer vaccines.

In some embodiments, the vehicles of the presently disclosed subject matter encompass and origami-folding RNA or an RNA origami as a therapeutic agent. In some embodiments, a TERCOM tail is added to the 5′-end and/or the 3′-end of the RNA origami. Thus, in some embodiments of the presently disclosed subject matter, polymers that have conformations that evolve with path (e.g., so to get to an end or functional conformation, the vehicle must take the correct path and, therefore, go through the correct intermediate conformation), the polymer is an RNA molecule that folds into a conformation. In some embodiments, a tail of the RNA gets eaten by nuclease while the active region conformation evolves with environmental path such that the vehicle must be on the correct path to both (a) not get digested to inactivity; and (b) establish the correct shape to be active. In some embodiments, where there is an active region that works by its shape and a tail, the active region does not change shape in a way that is path dependent, but the tail does. This could be in some embodiments a protein with a tail, or in some embodiments a polymer with a tail (e.g., an RNA with a tail), or in some embodiments a complex, such as but not limited to a complex with an protein that is the active therapeutic agent and RNA as a tail.

Thus, in some embodiments an RNA active region having a shape and/or an appended self-destruct fuse attached and in a liposome, optionally wherein the RNA conformation “evolves” with spatial targeting/TERCOM, the delivery vehicles of the presently disclosed subject matter offer reduced side effects for a given dose to the target site and/or allow increased dosing to the target for a given level of system dose/side effect. As such, for example, even if an RNA therapy such as a folded RNA that acts like a folded protein (as a “key” for a target site, or agonist/antagonist, etc.) has similar or even lower effect versus the corresponding therapeutic provided without the delivery vehicle of the presently disclosed subject matter (e.g., an insulin substitute), the ability of the delivery vehicles of the presently disclosed subject matter to active in a spatially-selective manner because of the TERCAM features (e.g., the self-destruct-fuse tail in liposomal delivery, or an environmentally-path sensitive RNA folding conformation trajectory that is spatially or environmentally path-dependent) can result in an improvement over the “naked” therapeutic agent due to the former having lower side effects.

In some embodiments, within a liposome there can be an RNA complexed with a protein in a way that makes the protein inactive. By way of example and not limitation, in some embodiments the RNA can comprise a first region that interacts with the active site of the protein to thereby inhibit its activity. The RNA can also comprise a TERCOM-self-destruct-fuse tail that in some embodiments is subject to degradation by a co-enveloped sensitive nuclease. In this manner, when this tail is degraded, the inhibitory region of the RNA can become non-functional, thereby leaving the protein active. In an embodiment such as this, the fuse can function as an activate fuse not a self destruct fuse since degradation of the tail de-inhibits (i.e., activates) the protein. In such an embodiment, the composition is designed such that the fastest consumption of the tail occurs when the composition is on the path to the target site as opposed to embodiments wherein the fastest consumption of the tail occurs when the composition is on a path to non-target sites (e.g., not on a path to the target set), thereby freeing it from the blocking RNA.

For this reason, existing folding RNA therapies and/or mRNA (or, generally, coding RNA) therapies (e.g., stock active regions) can be modified to include TERCOM-type functionality as set forth herein to give them enhanced selectivity and reduced side-effects even when the unmodified RNA therapies are not per se better than proteins or other such alternatives.

By way of example and not limitation, in some embodiments an RNA with an active site of fixed folding conformation can have a tail, the conformation of which evolves with environmental path (e.g., different parts of the RNA attract each other and the shape evolves along a path as the environment such as but not limited to salinity, pH, etc. evolves). In such an embodiment, only if the path to the target is taken does the tail reach a conformation that allows exposure of the active site.

In some embodiments, a polymer that is like a ball snake. In some embodiments, this is an RNA where the active site (the head) is a mock protein of fixed conformation and a tail balls around it, only unballing and revealing the mock drug if a specific environmental path is taken. The sequence for the RNA's tail would be computed by the methodologies outlined herein. In some embodiments, the sequence for its head would be that of a stock folding RNA mock of a protein.

Thus, in addition to modified mRNA vaccines, the presently disclosed subject matter can be applied to the whole category of folding RNA mocks of protein drugs that would not necessarily be particularly more effective than the proteins they mock, except that the TERCOM fuse tails of the type outlined herein can more easily be added to RNA to make the mocks spatially-targeted and therefore less likely to cause side effects. As such, in some embodiments RNA mocks of proteins with self-destruct fuses are encompassed by the presently disclosed subject matter.

By way of further example and not limitation, in some embodiments the presently disclosed subject matter relates to an RNA “mock” heparin (see Krissanaprasit et al., 2019), with a TERCOM-type self-destruct fuse tail in a liposome made of soy lipids with bacterial porins and a nuclease (e.g., RNase) inside the liposome, which is sensitive to nucleotide and/or ion concentrations. This can be similar to what is described in Salomon et al., 2020.

As a variation, a liposome formulation as described in Salomon et al., 2020 (DOTMA based) but with the addition of one or more porins can be employed. As another variation, the “active” RNA from Salomon et al., 2020, which is a vaccine-type mRNA not an folding “mock” protein-type, can be employed.

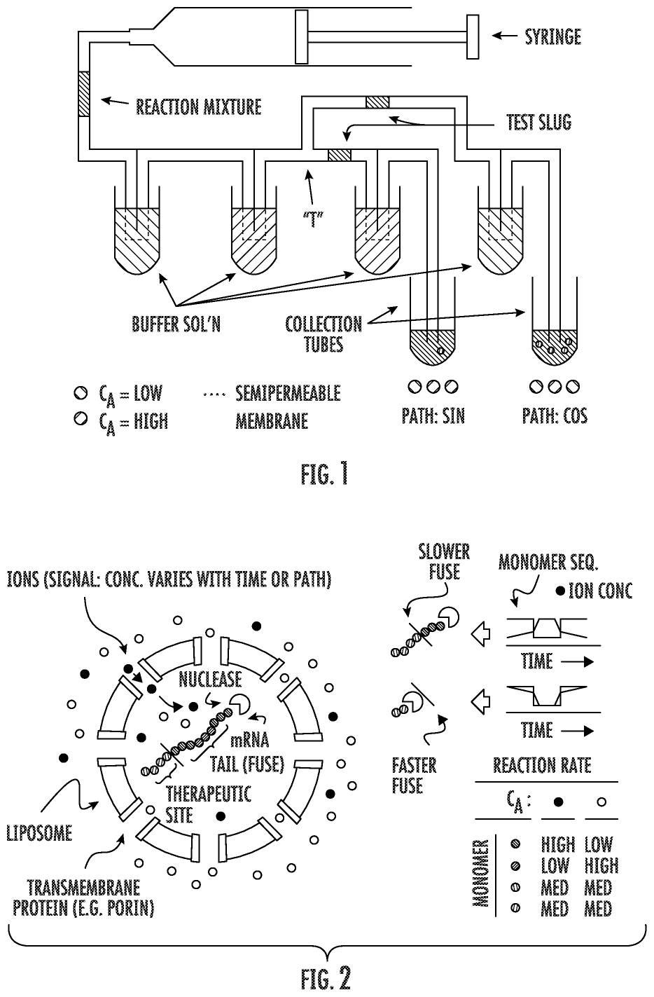

In some embodiments, a folding RNA mock heparin (see Krissanaprasit et al., 2019) can be employed. Other RNA mock proteins can also be employed, and one of ordinary skill in the art can add a TERCOM-type self-destruct fuse as disclosed herein to them. An exemplary embodiment is shown in FIG. 2.

For example, because folded RNA mock-insulin or mock-defensin can be easily modified to have TERCOM functionality as set forth herein, the presently disclosed subject matter can provide an attractive substitute for, for example, actual insulin or actual defensin because the TERCOM functionality can result in reduced side effects (e.g., by lower dosing at non-target sites). As such, the presently disclosed subject matter also provides RNA mock versions of protein therapeutics (either by encoding the same or acting as a mimetic) that have TERCOM functionality added by way of conformation evolution and/or spatially-sensitive self-destruct-fuse-tail sacrifice. Exemplary protein drugs and other drugs that can have RNA mock versions (e.g., made with RNA folding) that can be modified with TERCOM functionality include, but are not limited to insulin, defensin, novichoks, or in fact any other protein or non-protein drugs.

Also provided are chips that can evaluate selectivity of the presently disclosed nanoparticles.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Example 1

Reagents, equipment, and other supplies used for the experiment are detailed below.

Reagents. The list of reagents include: single-stranded DNA substrates; TdT enzyme and associated buffer, and ion solutions for the extension reaction; nucleotides for the extension reaction; a cleanup kit for the collected sample; DNA tails to be added via ligation in preparation for sequencing; and a T4 ligase enzyme kit plus associated chemicals, used to perform tail ligation in preparation for sequencing.

The following reagents are used during each run:

1×TdT reaction kit, large; Terminal Transferase reaction kit including TdT, 10× concentrated TdT buffer (composed of 50 mM Potassium Acetate, 20 mM Tris-acetate, and 10 mM Magnesium Acetate at pH 7.9 @ 25° C.), and 10× concentrated CoCl₂ (available as item M0315L, large, 2,500 units at 20,000 units/mL from NEB, Ipswich. Massachusetts, United States of America; also available as NEB item M0315S, small, 500 units at 20,000 units/mL; each run of an experiment uses 10 units TdT and 400 units buffer/ion solution); used to add nucleotides to ssDNA substrates during extension reaction; NEB item M0315L.

Optionally, 10×TdT reaction buffer; ten additional vials of 10× concentrated TdT buffer of type found in TdT reaction kit (composed of 50 mM Potassium Acetate, 20 mM Tris-acetate, and 10 mM Magnesium Acetate at pH 7.9 @ 25° C.); used in dialysis buffer.

Optionally, 10×CoCl₂ ion solution; custom order; ten additional vials of 10× concentrated CoCl₂ of type found in TdT react. kit; used in dialysis buffer.

1× nucleotide solution; large pkg. of mixed (A, C, G, T) dNTP nucleotide solution at 10 mM per nucleotide type (NEB item N0447L or N04475); used to provide monomers to the TdT extension reaction (NEB item N0447L).

1× substrate ssDNA oligo; oligo with Fluidigm common sequence 1, as per Bhan et al., 2019, CS1: 5′-ACACTGACGACATGGTTCTACA-3′ (SEQ ID NO: 1), purchased from ThermoFisher Scientific, Waltham, Massachusetts, United States of America; used as tails for sequencing process.

1× ssDNA/RNA cleanup kit; ssDNA/RNA clean/concentrator, Zymo Research D7010, size 20 preps; used to remove TdT, reaction buffer, cations, and free nucleotides from collected sample; Zymo Research; cat #D7010.

The following reagents are used when sequencing the samples created by the experiment:

1× tails oligo; oligo with Fluidigm common sequence two, as per Bhan et al., 2019, CS2 reverse complement: 5′-AGACCAAGTCTCTGCTACCGTA-3′ (SEQ ID NO: 2), purchased from ThermoFisher; used as tails for sequencing process.

1× T4 RNA ligase 1; T4 RNA ligase 1, 1000 units at 10,000 units per mL; used to blunt-end ligate tails to the 3′ end of the extension reaction product, as per Bhan et al., 2019 (NEB item M0204S).

1× reaction buffer for T4 RNA ligase; reaction buffer kit for T4 RNA ligase, at 10× conc., 3 mL; used in T4 RNA ligase reaction (NEB item B0216L).

1×PEG 8000; 500 g of polyethylene glycol 8,000 for biotechnology, H(OCH₂CH₂)ₙOH; used in T4 RNA ligase reaction; (item no. 97061-102 from VWR International, Radnor, Pennsylvania, United States of America).

1×1 mM ATP; Adenosine 5′-Triphosphate (ATP), 1 mL at 10 mM, storage −20° C., Mol. Wt. 551.2 daltons; used in T4 reaction for sequencing prep (NEB item P0756S).

1× Tris-HCL (ph 8.0); Tris-HCL, 25 mM (pH 8.0), (VWR item PAA2641).

1×EDTA; EDTA, free acid ultrapure, 500 gm (or substitute); used to halt T4 reaction in NEB standard protocol (VWR item 97061-406).

The following reagents are also used when sequencing the samples created by the experiment:

1×PCR mix; Phusion High Fidelity PCR Master Mix with HF Buffer, 2×, 100 reactions, 50 uL volume; used in PCR step of sequencing (NEB item M0531S).

The TdT reaction kits are expensive due to the TdT, but for the present EXAMPLES, the ratio of TdT reaction buffer solution required to TdT required is much greater than comes in a kit (roughly 10 times more required) due to it being used in the dialysis buffer. There are a number of options to acquire the extra reaction buffer: it can be cannibalized from TdT kits whose TdT is discarded; it can be purchased from NEB as a custom order; or it can be prepared from constituent reagents as set forth herein below.

The baseline experiments use 10 units of TdT per run yielding a 50 μL collect. There are four dialysis buffers, and each requires 10 times the volume of a collect, so each run uses 10× (1+4×10)=410 unit equivalents of TdT reaction buffer.

The tail ligation uses NEB “Protocol 3” available at the website of NEB for “ligation of an [DNA] oligo to single stranded DNA using T4 RNA ligase 1 (NEB item M0204)”, which has the following steps:
(1) Set up a 20 μL reaction as follows: 1× reaction buffer; 25% (wt/vol) PEG 8000; 1 mM hexamine cobalt chloride (optional), 1 μL (10 units) T4 RNA ligase, 1 mM ATP;
(2) incubate at 25° C. for 16 hours;
(3) stop the reaction by adding 40 μL 10 mM Tris-HCL pH 8.0, 2.5 mM EDTA.
   Tri-HCl may be prepared by dissolving 121.14 gm Tris in 800 mL distilled water. The pH is adjusted with an appropriate volume of HCl, and the final volume is brought to 1 L with deionized water. The solution is sterilized, for example, by autoclaving and stored at room temperature before it is used to halt T4 reaction protocol (described herein)

Equipment:

2×CMA 7 Probe 3-pk; micro-dialysis probes, package of three, with integral interconnect tubes, 7 mm shaft, and 1 mm membrane having cut-off of 60,000 Daltons; used for experiment (Harvard Apparatus, Holliston, Massachusetts, United States of America).

2× micro tee; micro “tee” interconnect for probe tubes; used to branch or join interconnect lines (Harvard Apparatus item CMAP000043.

1× connecting tubes, 10-pk; connecting tubes, 10 pc. per package; used to connect the inlet lines, outlet lines, etc. of the probes, syringe, etc. (Harvard Apparatus item CMA3409500).

1×CMA-130 stand; probe stand (Harvard Apparatus model CMA-130) including three CMA-7-compatible clips and three Eppendorf-tube holders, plus one additional clip and holder (Harvard Apparatus model CMAP000136); used for holding probes and test tubes during experiment (Harvard Apparatus items CMA8309104 and CMAP000136).

1× syringe, 1 mL; syringe, 1 mL volume; used to supply test mixture to tubes (Harvard Apparatus).

1× dry booth; MICRO-MAKE™ Doctor DryBooth paint drying booth, with 1082 cu. in. volume and at 100-105T on low heat setting; used as a laboratory incubator for test rig (item #88043 from Micro-Mark, Berkeley Heights, New Jersey, United States of America.

1× chill block; freezer block for micro-centrifuge test tubes, delivers 0° C. for 4 hours in room temp. environment; used to stop reaction at point of collection (item #215587 from Carolina Biological Supply, Burlington, North Carolina, United States of America).

1× partial immersion thermometer; thermometer with 6-7 mm diam, 6 in. length, −10 to 110° C. range, partial immersion type; used to record collection point temperature (Carolina Biological Supply item #745461).

1× incubator thermometer; thermometer with 90-110° F. range in IT steps, for use in incubators; used to record temperature in heating booth (Carolina Biological Supply item #701240).

1× indoor thermometer to record room temperature (Carolina Biological Supply item #701240).

1× vise; mini vise that holds odd shapes gently yet securely, with 1 in. capacity; used to hand-push syringes at a controlled rate (Micro-Mark item #86816).

1× electronic balance; economical compact electronic balance (Carolina Biological Supply item #702065).

1× countdown timer; single-channel electronic timer with count time of up to 99:59, up or down, three buttons; used as countdown timer (Carolina Biological Supply item #702679).

1× stopwatch; electronic stopwatch-style student timer; used to track elapsed time (Carolina Biological Supply item #973033).

1× mini PCR unit; miniPCR mini8 thermal cycler, rainbow; used for PCR step of sequencing procedure (minipcr, Cambridge, Massachusetts, United States of America).

1× Parafilm; sealing film, roll of 4 in.×125 ft.; used for sealing test tubes during handling (Carolina Biological Supply item #215600).

1× Eppendorf tubes, 1000-pk; microcentrifuge tubes, 1.5 mL, sterile, polypropylene, clear/natural, package of one thousand; used for holding buffers, etc. (Carolina Biological Supply item #215236).

1× working tube rack, 7-pack; working microtube rack for 1.5 mL and 0.5 mL tubes, 40 (32+8) seats per side, 7-pk; used for holding micro tubes during preparation (Carolina Biological Supply item #215575).

1× glass beads; 150 pc. per 1 oz pack; used as inert filling to reduct liquid volume in 1.5 mL tubes (Carolina Biological Supply item #215821).

1× storage tube rack; STYROFOAM® microtube storage racks, 100 wells; used for long-term storage of micro test tubes (Carolina Biological Supply item #215563).

1× micro pipette; 2-20 μL micro pipette; used for preparation of reagents (minipcr item QP-1001-01).

Alternatives and Substitutes. The experimental equipment described above is selected for a middle budget and features a mix of expensive miniature components and inexpensive, manual equipment. The cost to assemble the test apparatus may be reduced by using low-cost substitutes for expensive components. The ease and reliability with which the experiment can be performed may be increased by substituting expensive automated equipment for inexpensive manual equipment. A list of alternatives and substitutes follows.

Also, the reagent list above assumes that the dialysis buffer is made by adding nucleotides to custom ordered volumes of the buffer and CoCl₂ solution provided in the TdT reaction kits supplied by NEB. In the event that these cannot be ordered alone, the dialysis buffer may be prepared from scratch using stocks of Potassium Acetate, Tris-acetate, Magnesium Acetate, and CoCl₂. A list of ingredients for hand-assembled dialysis buffers is provided below.

Low-Cost Manual Substitutes for Expensive Equipment and Components.

2× positioning stand; “Triple Grip Third Hand” stand with alligator-type spring clamps; substitutes for specialized probe stand; used to hold dialysis buffer test tubes with semi-submerged probes (Micro-Mark item #21120).

1× balance; triple beam balance by Carolina Biological Supply, 610 gm capacity, 0.1 gm readability (or use 1× balance; triple beam balance, model 750-S0 by Ohaus, 610 gm capacity, 0.1 gm readability; Carolina Biological Supply item #702150); substitute for electronic balance; used for measuring reagents (Carolina Biological Supply item #702020).

More Expensive Automated Substitutes for Inexpensive Equip. And Components.

1× electronic balance; electronic balance, 150 gm, readability 0.01 gm; used for measuring dry reagents and for measuring collected mass (Carolina Biological Supply item #702010).

1× precision balance; precision digital balance with 0.010 g readability of up to 1.2 kg; substitute for scale, except can measure collected mass mid-experiment since it can tare a chill block; used to measure mass of collected streams (Carolina Biological Supply item #702542).

1× lab incubator; incubator with 0.7 cu. ft. capacity; substitute for heating booth; used to keep test rig at incubation temperature for reaction (Carolina Biological Supply item #701296).

1×CMA 470 refrigerated micro-fraction collector (Harvard Apparatus item CMA8002770); automated sample collector, with refrigeration to 6° C.; substitute for chill block; used to collect sample streams.

1× CM 142 micro-fraction collector; automated sample collector, unrefrigerated; substitute for chill block or refrigerated collector; used to automate collection of sample streams, but collects at room temperature (Harvard Apparatus item CMA8381143).

1×CMA 110 liquid switch with tubing kit; three-line manual liquid switch used to avoid introduction of air bubbles when switching between fluids; substitute for junction at syringe; used when switching between prime or flush streams and test stream (Harvard Apparatus item CMA8308200).

1×CMA 402 two channel syringe pump with two independent channels; syringe pump that rates from 0.1 μL/min to 20 μL/min (Harvard Apparatus item CMA8003100), CMA 402 micro-dialysis syringe pump with accessory kit); substitute for manual vise; use to push fluids from syringe at controlled rate (Harvard Apparatus item CMA8002773).

1× mini PCR battery pack; miniPCR power pack, 20,000 mAh Li-ion battery; used for cord-free operation of mini8 (MiniPCR item QP-1000-13).

Raw materials for hand-prepared dialysis buffer:

1×500 gm potassium acetate; 500 gm of crystal, reagent-grade CH₃CO₂K, potassium acetate; used in hand-prepared dialysis buffer as partial substitute for reaction buffer provided in TdT kits (Carolina Biological Supply item #882540).

1×100 gm tris-acetate; 100 gm of tris-acetate; used in hand-prepared dialysis buffer as partial substitute for reaction buffer provided in TdT kits (VWR catalog no. 97061-174).

1×500 gm magnesium acetate tetrahydrate; 500 gm of magnesium acetate tetrahydrate; used in hand-prepared dialysis buffer as partial substitute for reaction buffer provided in TdT kits (VWR catalog no. 97061-060).

1×500 mL cobalt chloride solution; cobalt chloride hexahydrate, COCl₂·6H₂O (available as solution, 0.1 M, laboratory grade, 500 mL, Carolina Biological Supply item #854955); also available as powder, reagent grade, 100 gm, Carolina Biological Supply item #854959); used in some reaction or dialysis buffers.

Experimental Setup.

The micro-dialysis probe stand is set up with four stations, each having a test tube and a probe, with each probe tip projecting into its test tube such that its tip is submerged when that test tube is filled.

The entire assembly is placed inside the convective heating booth (see Note 2 below) and maintained at or near 37° C. (approx. 100° F.) throughout a run of the experiment; this is done to keep the dialysis buffers and the test streams within the probes and interconnects at the incubation temperature.

The discharge streams are collected in test tubes placed in the chill block. The chill block keeps the collected discharge at a temperature of 0° C., halting the extension reaction upon collection.

The incubator thermometer is placed inside the heating booth; it is used to monitor the incubation temperature. The partial-immersion thermometer is placed in a saltwater- or ethanol-filled test tube mounted in the chill block; it is used to monitor the collection temperature. The indoor thermometer is placed near the test rig; it is used to monitor the room temperature, which the syringes are presumed to be at. The countdown timer is set to 20 minutes; it is used to provide a reminder to reset the heating booth timer every 20 minutes. The stopwatch is used to track the time elapsed since the start of the experiment; it is started when injection fluid is first pushed from its syringe and stopped when injection fluid is last pushed from the same.

The volume of dialysis buffer in each test tube should be at least ten times that of the collected volume (e.g., 0.5 mL=10×50 μL) in order to assure that diffusion from a probe stream into a dialysis buffer does not significantly alter the compositions of the latter. Depending on the probe used, more may be needed; if the water line of a 1.5 mL test tube filled with 0.5 mL of dialysis buffer cannot submerge the probe tip, it should be raised by: adding a sterile glass bead to the 1.5 mL test tubes; using more dialysis buffer in the 1.5 mL test tubes; or switching to a smaller test tube size, such as 0.5 mL. All of the dialysis buffer test tubes should contain the same volume of buffer.

As an alternative to being placed within a convective heating booth or incubator, the apparatus may be blown on by a blow dryer, placed under a heat lamp, blanketed by a heating pad, placed in a warm water bath (test tubes must be sealed in this last case), etc. In such instances, the objective of the heater remains: to maintain the rig at the incubation temperature during the course of the experiment.

Thermocouples may be used as an alternative to manual thermometers to monitor the incubation and collection temperatures, and their outputs may be recorded manually or automatically.

Reactants.

The reactants used in the experiment are described below.

The priming mixture is prepared as follows:

1.00 mL of deionized water is drawn into the 1 mL prime/flush syringe and kept at room temperature.

The probes and interconnects are primed before each experiment to eliminate air and flushed at the end of each experiment to rinse away remaining chemicals. Water is used. However, it must be free of ions and enzymes so as to avoid disrupting the DNA extension reaction. So, per the recommendation of vendors, deionized water suitable for biotech use or other special water is used.

The dialysis buffers are prepared as follows: 0.050 mL of fresh 10× conc. TdT buffer is added to each of four 1.5 mL clean test tubes.

0.050 mL of deionized water is added to two of the four test tubes.

0.050 mL of fresh 10× conc. CoCl₂ solution is added to the other two test tubes.

0.050 mL of 10 mM nucleotide solution is added to each of the four test tubes.

0.400 mL of deionized water is added to all four test tubes.

The result of preparation is two tubes of dialysis buffer containing 0.50 mL of fresh 1× conc. plain (“Mg only”) reaction buffer (composed of 50 mM Potassium Acetate, 20 mM Tris-acetate, 10 mM Magnesium Acetate) and 1 mM ea. nucleotides; and two containing 0.50 mL of fresh 1× conc. enhanced (“Mg+Co”) reaction buffer (which has an added 0.25 mM Co²⁺) and 1 mM ea. nucleotides.

The injection mixture is prepared as follows:
0.450 mL of fresh 10× conc. TdT is added to a clean 1.5 mL test tube.
0.050 mL of substrates are added to the test tube
0.500 mL of deionized water are added to the test tube

The result should a test tube containing the TdT enzymes, plain reaction buffer (“Mg-only”), and the ssDNA substrates, but no nucleotides which to append (these become available later, at the first micro-dialysis probe). The injection mixture is kept refrigerated or at room temperature to forestall any reactions until the time of use. The preparation recipe given here is for 1 mL of injection mixture, but only 50 μL is required for any one experiment.

Experimental Procedure.

The procedure for a single run of the experiment is as follows.

The experimental charges are drawn into the flush syringe and the injection syringe from their respective preparation vehicles. The apparatus is primed and brought to thermal equilibrium. The test tubes containing the dialysis buffers are mounted in their respective locations on the probe stand, with each probe's tip submerged in its corresponding dialysis buffer. The probe rig is placed inside the heating booth. The chill block is removed from the freezer and placed near the heating booth. The collection test tubes are weighed and their empty mass recorded. The collection test tubes are placed in the chill block. The heating booth is turned on. The temperature of the probe rig is allowed to rise to the incubation temperature of approx. 37° C. or approx. 100° F. The syringes are connected to the apparatus. The probe bodies and interconnect lines are primed by discharging approximately 0.10 mL of priming/flushing liquid from its syringe.

The stopwatch and timer are started, and the injection solution is discharged from its syringe at an average rate of approximately 1 μl per minute; injection is allowed to continue for 60 minutes.

After 60 minutes, the collection lines are connected to the collection test tubes. The injection solution continues to be discharged from its syringe at an average rate of approximately 1 μl per minute; injection is allowed to continue for an additional 60 minutes.

Throughout the injection period, the temperatures inside the heating booth and cooling block are monitored and periodically recorded. The heating booth operates on a 25 minute countdown timer, so every twenty minutes, its timer is reset.

After the additional 60 minutes have elapsed, the experiment is stopped. The collection tubes are relocated to a waste collection tube or beaker. The probes and interconnects are flushed with a 500 μL push of water from the prime/flush syringe.

The mass collected in the filled collection test tubes is measured and recorded; their recorded empty mass is subtracted to give the collected mass in each.

The samples in their collection test tubes are processed and sequenced.

Discharge of the injection solution from its syringe may be manual or automated. If performed manually, the syringe may be placed in a small vise which is closed slowly by performing a partial turn of its screw at a frequency (e.g., once per minute) and amplitude (e.g., quarter turn) sufficient to give the desired avg. flow rate but not so great as to cause a poor capture ratio at the probe tips or so slow as to cause unwanted upstream diffusion in the line.

The recommended model of heating booth runs on a 25 minute timer, which should be reset every 20 minutes (three times during the experiment) to ensure continuous and stable incubation. If available, a more expensive laboratory incubator may be used in lieu of the recommended model.

The recommended model of heating booth maintains a temperature of approx. 100° F. (approx. 37.7° C.) when set to its low setting, so this setting should be used for the experiments. If available, a more expensive laboratory incubator may be used in lieu of the recommended model.

Operating Concept. The operation of the apparatus during an experiment is as follows:

Initially, the apparatus has water in its primed lines, nucleotides and reaction buffer in its dialysis buffers, and terminal transferase (TdT) and substrates in the syringe; the micro-dialysis probes, dialysis buffers, and interconnects are at the incubation temperature; the syringe is at room temperature; and the collection tubes are chilled. No reactions take place because the nucleotides are kept separate from the transferase and the syringe contents.

The experiment begins when the syringe begins to push the transferase/substrate mixture into the primed lines, where it acquires the incubation temperature. The mixture makes its way to the tip of the first probe, which is submerged in the first dialysis buffer (e.g., “Mg-only”, with nucleotides). Here, the stream picks up the solutes and nucleotides required for reaction via diffusion through the semipermeable membrane, and the extension reaction begins. The transferase enzyme and substrates remain in the line, as their size is at or above the cut-off mass for the membrane. The stream leaves the tip and proceeds through the interconnect lines to the next probe. During this transit, the extension reaction continues under the conditions arranged by the transit of the first tip.

When the stream reaches the second probe tip, it dumps the solutes and nucleotides of the first buffer and exchanges them for those of the second dialysis buffer (e.g., “Mg+Co”, with nucleotides); this occurs via passive diffusion through the semipermeable membrane. The stream leaves the tip and proceeds through the discharge line to the “tee”, where it splits into two streams: one that flows into the third probe and one that flows into the fourth probe. The extension reaction continues inside the interconnect lines and tee during this transit, under the conditions arranged by the transit of the second tip (e.g., “Mg+Co”).

When the branching stream that passes from the “tee” to the third probe reaches that probe's tip, it dumps the solutes and nucleotides of the second buffer and exchanges them for those of the third (e.g., “Mg+Co”, with nucleotides); this occurs via passive diffusion through the semipermeable membrane. It then proceeds to the collection point via the discharge line. The extension reaction continues inside the interconnect lines during this transit, under the conditions set up by the transit of the third tip (e.g., “Mg+Co”).

Similarly, when the branching stream that passes from the “tee” to the fourth probe reaches that probe's tip, it dumps the solutes and nucleotides of the second buffer and exchanges them for those of the fourth (e.g., “Mg+Co”, with nucleotides); this occurs via passive diffusion through the semipermeable membrane. It then proceeds to the collection point via the discharge line. The extension reaction continues inside the interconnect lines during this transit under the conditions set up by the transit of the third tip (e.g., “Mg+Co”).

Upon reaching the collection points, the reactions in the two streams will cease due to the temperature reduction caused by the cooling block (or refrigerated collector). Depending on the length of the collection lines, the streams will discharge at a temperature between the incubation temperature of the rig and the ambient temperature, but they will cool quickly in the test tube.

The entire transit from injection point to collection point take approximately 60 minutes, so the collected discharge does not start to represent the slug of experimental reactants until 60 minutes have elapsed; it is preceded by the priming slug. After 120 minutes have elapsed, the test slug has completed its passage and the probes and interconnect lines have left in them a slug of fluid that served as a pusher. Under an alternative approach, the experiment would be run for an extended period, with several 50 uL collects made during that time.

During the course of the experiment, it may be desirable to take micro samples from the collected discharge. This may be accomplished using a micro-pipette to draw samples from the collection tube, or by switching the discharge lines to new test tubes also placed in the cooling block, or by some other method. If an automated collector is used, it may be programmed to perform collects.

The exemplary experiment outlined above is for a demonstration of path-sensitive synthesis using an unencapsulated reaction mixture. The exemplary experiment outlined in EXAMPLE 2 demonstrates path-sensitive synthesis with the reaction mixture encapsulated in a liposome.

### Example 2

The following materials and supplies are incremental ones, beyond those used in the baseline experiment, which are required by the optional additional experiment.

Reagents. The list of reagents for the optional experiment include: lipids, to form liposomes; chloroform, used as a solvent during liposome prep.; transmembrane proteins that functions as nonselective ion passive transport ion channels, used to make liposomes permeable to regulating ions; detergent, used during preparation of the proteins; dehydrate-rehydrate solution, used during liposome prep; and biobeads, a kit used for cleaning (detergent removal) during liposome prep.

1×TRPM4; TRPM4 over-expressed Lysate (adult normal) by supplier Novus Biologicals (NBL1-17336); used as ion channel in liposomes.

1× detergent; detergents used for protein purification (e.g., 1% lauryl maltose neopentyl glycol (LMNG), 2% n-dodecyl-B-D-maltopyranoside (DDM) or 1% digitonin).

1× inhibitor; flufenamic acid (FFA) used in some experiments at 20 μM to inhibit TRPM4.

1× potassium chloride (for D-R); potassium chloride solution, 0.5 M, 500 mL, laboratory grade (or substitute biotechnology grade from VWR or potassium chloride, granular, ACS grade, 500 gm, (Carolina Biological Supply item #882910); used to make D-R fluid (200 mM KCl, 5 mM HEPES, pH 7.2) for liposome making (Carolina Biological Supply item #882914).

1×HEPES (for D-R); HEPES, Sterile, 1 M, pH 7.3, biotechnology grade, 100 mL; used to make D-R fluid (200 mM KCl, 5 mM HEPES, pH 7.2) for liposome making (VWR cat. no. 97064-360).

1× soybean azolectin lipid (see note 1 below; assumed to be “Soy Phospholipid Mixture (Soy PC 3.8 mg, Soy PE 3.0 mg, Soy PI 1.8 mg, Soy PA 0.7 mg, Soy LPC 0.7 mg with 1 pct BHT),” SKU 690050, offered by Avanti Polar Lipids, Inc., Alabaster, Alabama, United States of America in powder form and in chloroform as 1×10 mg as 1 mL of with 10 mg/mL.

1× chloroform; chloroform, 1 L, purified 98-100% (VWR chloroform, 99.8 pct, stabilized for biotechnology, cat. no. 97064-678); used in making liposomes (VWR cat. no. MK443210).

1× biobeads; biobeads SM-2 kit; used during liposome prep.; Bio-rad Laboratories, Inc, Hercules, California, United States of America).

1× argon (see Note 2 below); argon gas under pressure in can (Bloxygen, San Luis Obispo, California, United States of America).

Equipment.

1× centrifuge (see Note 3 below); microcentrifuge, fixed-speed, 10,000 rpm, 4,800 g, GYRO™ by miniPCR; used during liposome prep (miniPCR item QP-1800-01).

1× petri dish; petri dish, 100 mm×10 mm, PYREX® 3160; used during liposome prep. (Carolina Biological Supply item #741160).

1× microscope slides; microscope slides, glass, standard, 25×75 mm, 0.8-1.0 mm; used during liposome prep. (Carolina Biological Supply item #631920).

1× desiccator; non-vacuum desiccator, six-in.; used during liposome prep. (Carolina Biological Supply item #742972).

Misc. Supplies.

1× filter paper; filter paper, quantitative, 7 cm, pkg. of 100-circles; used during lip. prep. (Carolina Biological Supply item #712807).

Alternatives and Substitutes.

The following optional equipment can aid in liposome prep. An extruder provides the option to form uniform-sized liposomes (see Note 4 below). A rotovap provides the option to dehydrate liposomes (see Notes below).

1× extruder; mini extruder set with holder/heating block, 610000 (available as SKU 610000-1EA: instructions, mini-extruder, two 1000 μL syringes, 100 0.1 um pore polycarbonate membranes, 100 filter supports, used in liposome prep. (Avanti Polar Lipids, Inc. item 610000-1EA).

1× rotary evaporator (e.g., Mophorn 2 L rotary evaporator R201D, 5-120 RPM, 1 L rotary bottle or Lanphan RE-501 rotary evaporator; used in liposome prep.

Constantine et al., 2016 calls for soy azolectin lipids from Avanti, but Avanti Polar Lipids, Inc. does not list product with that exact name. So, for construction of the reagent list and pricing, the offering “Soy Phospholipid Mixture (Soy PC 3.8 mg, Soy PE 3.0 mg, Soy PI 1.8 mg, Soy PA 0.7 mg, Soy LPC 0.7 mg with 1% BHT)” was used.

Constantine et al., 2016 also calls for a nitrogen stream during the first dehydration step of liposome prep. If nitrogen is unavailable, canned argon (Bloxygen) was substituted here.

Constantine et al., 2016 specifies ultracentrifugation in a 43K rpm centrifuge (Beckman 50.2Ti) for 30 min.

Constantine et al., 2016 does not use extruded liposomes, but Tanner et al., 2010 does.

Tanner et al., 2010 used a rotovap for liposome prep., although Constantine et al., 2016) does not.

Experimental Setup.

The experimental setup for this experiment is identical to that for the experiment set forth in EXAMPLE 1, except for changes related to the use of liposomes to encapsulate the polymer-synthesis formulation.

The liposomes encapsulate the formulation from the experiment of EXAMPLE 1 at the same concentration employed, and the liposomes themselves are suspended in the injection solution at same concentration that they are found in after rehydration (i.e., 2 mg of lipid added to each 20 uL of solution). So, the collect should collect the same volume of reaction mixture as in the experiment of EXAMPLE 1, and that reaction mixture should be the same.

For this experiment, the preparation of the liposomal injection mixture, composed of liposomes charged with reaction mixture and hosting passive transport channels in the bilayer, follows the same procedure described in Constantine et al., 2016 (Supporting Information) under the title “Preparation of Proteoliposomes.” However, some modifications are made to ensure that the liposomes are charged with the proper reaction mixture. Specifically, the rehydration step that forms the liposomes uses a solution that contains the contents of the injection mixture from the baseline experiment (TdT and ssDNA substrates), and nucleotides, at the required concentration, so as to ensure that the liposomes encapsulate the requisite parts of the reaction mixture.

Because a larger volume of injection liquid is pushed from the syringe during a run of the optional experiment, the there is a greater prospect of “poisoning” the dialysis buffers with ions from the sample stream (see discussion in main text). In the baseline experiments, the volume of each test tube of dialysis buffer was made at least ten times the sample volume (of 50 μL) to avoid such “poisoning”. In the case of the optional experiment, which sees a push of roughly 7×50 μL or 350 μL, a factor of 10:1 would require 3.5 mL of dialysis buffer per test tube. This would require either the use of a larger tube, the refreshing of the contents of a tube mid-experiment, the accepting of a smaller ratio of dialysis buffer to injected volume, or a reduction of the injected volume. To minimize disruption to the experimental apparatus, it is recommended that 1.5 mL of buffer be used in 1.5 mL tubes, which will give a factor of 1.5 mL/0.350 mL=4.5:1, and that the effect of this reduction be addressed with post-processing of the data. However, the other options are also viable. One that is attractive is to inject a smaller total injection volume, such as 0.14 mL, 0.050 mL, or 0.020 mL, giving a correspondingly smaller volume of collected reaction mixture.

If necessary for liposome formulation, the components of the D-R mixture of Constantine et al., 2016 (e.g., KCl, etc.) may also be included provided these can later be removed via diffusion through the passive transport channels or, if they remain encapsulated, do not affect the extension reaction.

Experimental Procedure.

The experimental procedure is identical to that of the experiment described in EXAMPLE 1 except for the following changes: (1) the injection syringe is filled with the liposome-encapsulated formulation in lieu of the unencapsulated formulation used in the baseline experiment; (2) a larger total volume is drawn into the injection syringe so that the discharged and collect volumes of the encapsulated formulation are as near to 50 μL of unencapsulated discharge as can be conveniently achieved; (3) the discharge rate is kept the same, since there is still a need to equilibrate the various parts of the streams within the probes and interconnects with their appropriate dialysis buffers, but the push time is lengthened due to the larger push volume; (4) an extra step is introduced as part of the sequencing preparation wherein the collected synthesized ssDNA strands are released from the liposomes that encapsulate them, by breaking the liposomes apart with a detergent or by some other appropriate mechanism, prior to the cleanup step that extracts only the ssDNA.

Operating Concept. The operating concept for the optional experiment is similar to that of the experiment of EXAMPLE 1, except that the reaction formulation is encapsulated in liposomes, which keep the extending substrates and the polymer synthesis enzyme (e.g., TdT) separate from the fluid that surrounds it within the probes, interconnects, and other flow passages, all the way to the collection point.

As with the experiment described above, the fluid environment in the tube which immediately surrounds the liposomes evolves along the path length due to exchange with the various dialysis buffers across the semipermeable membranes (i.e., at the probe tips). The liposomes have embedded within their lipid bilayer nonselective passive transport channels (e.g., TRPM) which allow the ions from the surrounding stream to reach the sites of polymer extension (e.g., the sites of the DNA extension reaction). However, the polymer strands (e.g., extended ssDNA) and the reaction enzymes (e.g., TdT) are too large to escape through these pores, so the reactions occur only within the liposomes and the polymer strands remain within the liposomes throughout the time of the transit from the injection point to the collection point. Since the nucleotides may also be too large to pass through the nonselective passive transport channels, these are also added to the charge stored within the liposome. As with the baseline reaction, the differing series of environmental conditions along differing pathways in the branching flow network cause the synthesized polymer strands to differ in their monomer sequence along different paths through the flow network, so the monomer sequences of the collected strands should correlate with their collection point despite the reactants that formed them being the same and despite the injection point for those reactants being the same.

In the experiment described here, the 50 μL of injection formula (e.g., TdT and ssDNA substrates) and a corresponding supply of monomers at the desired concentration (e.g., with nucleotides) are found only in the charges encapsulated within the liposomes. Since the liposomes are suspended in the stream flow at some concentration (i.e., liposomes per unit volume), and the bilayer accounts for some fraction of the volume of a charged liposome, the volume of encompassed charges will only account for some fraction of the volume of a slug of the stream. As a result, to collect a given volume of reaction formula, it will be necessary to inject some multiple of that volume of injection mixture.

Specifically, as noted below, the charge volume was estimated to account for 50% of the volume of a formed liposome (the other 40% being the bilayer) and, at 2 mg of lipid per 20 μL of solution, the liposomes were estimated to account for 23% of the volume of a slug of injection mixture (fill fraction of 23%) so the charge volume (a.k.a. the reaction volume) was estimated to account for 14% of the slug volume (i.e., the injection volume). So, the ratio by volume of liposomal mixture injected to reaction mixture collected is 1/0.14=7.1˜7, and the collection of 50 μL of reaction mixture and its products requires the injection of 7×50 μL=350 μL of liposomal injection mixture.

The above estimates for the fraction by volume of liposomes in the liposome mixture and of the fraction by volume of charge in a liposome were developed as follows. If the fineness (length-to-diameter ratio) and particle mass of a molecule or particle is known or can be estimated, and if its density as a liquid is known (and also assumed to be near its solid mass, so both may be called a condensed density), then the particle's effective diameter may be estimated from its mass by equating the product of its liquid density and volume with its particle mass. So, ρ(l/d·d) (π/4 d²)=M or d={4/π (M/φ/(l/d)}^{1/3} and 1={4/π/ρ}^{1/3} {l/d}^{2/3}; here, d is the effective diameter of the particle, l/d is its length-to-diameter ratio, M is its molecular mass, and ρ is its liquid mass density. If the diameter of a unilamellar liposome is known and its bilayer is treated as a thin shell with thickness assumed to be twice the length of a constituent molecule, then the fraction of the liposome's volume that is accounted for by the bilayer shell is f_{b}=(πD²t)/(π/6 D³)=6t/D with t=2l, and that accounted for by the charge (volume contained by the bilayer) is f_{c}=1−f_{b}; here, D is the diameter of the liposome, t=2l is the thickness of its bilayer, with l the length of one of the bilayer's molecules. Finally, the fraction by volume of liposomes in the liposome mixture may be found from the liquid density (liquid density, assumed approx. equal to the solid density) of lipids and mass of lipid added per unit sample volume by requiring pfₐf_{b}=MC_{A} so that fₐ=(MC_{A})/(pf_{b}); here, MC_{A} is the concentration by mass of lipids in the sample volume, ρ is the condensed or liquid density of the lipids, f_{b} is the fraction by volume of a liposome accounted for by its bilayer, and fₐ is the fraction by volume of a sample of the liposome mixture accounted for by the liposomes. An exemplary testing apparatus is provided in FIG. 1.

Assuming M=775 gm/mol with 1 mol=6.02×10²³ particles (see U.S. Patent Application Publication No. 2009/0275031 for “Soy PC”), assuming ρ=1.049 gm/mL, estimating l/d=5 (by inspection of the chemical formula for soy-based lipids; see U.S. Patent Application Publication No. 2009/0275031 for “Soy PC”), and assuming D=0.100 um (the pore diameter used by the extruder in Drake, 1988), one finds f_{b}=40% and f_{c}=1−f_{b}=60%. Assuming MC_{A}=2 mg per 20 π and that ρ and f_{b} are as given just above, fₐ=23%.

Through the underlying physics of the reaction, the spatial or temporal sampling resolution that can be achieved by noisy chemical recording in DNA is related to the number of strands whose sequences are averaged by the sampling process.

Suppose that, as in the experiments considered here, the addition of one particular species of monomer is much more sensitive to the presence of a particular ion and that the incorporation rates for the difference species are other wise roughly equal. In that case, the polymer may be considered to be composed of two general species of monomer: (1) the sensitive species, represented as a binary one (“1”), a coin flip that lands “heads” (“H”), or a the solute species of a dilute solution (“A”); and (2) the nonsensitive species (a.k.a. “background species”), represented as a binary zero (“0”), a coin flip that lands “tails” (“T”), or the solvent species of a dilute solution, (“B”).

If the strand grows to a length N, then, when the n-th monomer is added, the probability of it being one of the former type will be Pₙ, and that of it being one of the latter type will be (1−Pₙ); here n∈[1,n] and n≈t/T≈s/(uT), where T is the duration of the extension reaction, t is the time since the start of the extension reaction, s is the streamwise distance traveled along a path featuring a spatially-varying environment during t, (if such travel is a feature of the experiment), u is the average speed of that traverse, and Pₙ=f(s)ds, where f(s) is a probability density function. The probability Pₙ will depend on the local rate of incorporation of monomers, and that rate will depend on some mechanism that functions as a sort of “valve” (see U.S. Patent Application Publication No. 2009/0275031); examples of such “valves” include: (1) temperature-sensitive pores that regulate access to a supply of each species of monomer and, therefore, the local concentration in solution of each monomer; (2) a reaction rate constant that is sensitive to ion concentration, temperature, or some other such environmental variable; or some other such mechanism. In the discussion that follows, it will be assumed that a reaction rate sensitive to ion concentration functions as the “valve”, as occurs in both the baseline and optional experiments described, however the results that are derived will hold generally. If the environmental conditions are treated as taking on during a monomer addition interval only one of two different possible values, a baseline value and a value that includes a stimulus (e.g., “Mg ions only” and “Mg+Co ions”), so that the relative reaction rate (or the setting of some other such “valve”) takes on during that interval only one of two different possible values (e.g., “15% higher relative to that of background species” and “equal to that of background species”), then Pₙ may be modeled as taking on one of two different values, P_{L}=P₀=P̅−P′ and PH=P₀+2P′=P̅+P′; here, P₀ is the probability of a “heads” or “species A” add during interval dtₙ when no stimulus is present, ΔP/P₀=2P′/P₀ is the percentage increase in the probability of adding the sensitive species (“species A”) that occurs when the environmental stimulus is present, and P̅=P₀+½ΔP is the unweighted arithmetic average of P_{L} and P_{H}.

Now, suppose that the value of Pₙ that existed at some time tn is estimated by way of a hypothesis test. Suppose that the null hypothesis H₀ is that the Pₙ=P_{L} and the “accept” hypothesis, H₁, is that Pₙ=P_{H}. Suppose that the decision to accept or reject is made by first taking a window m monomers wide about monomer n=N·tₙ/T of each of the i-th strands, computing kᵢ/m, the fraction of the monomers in that window that are “binary one” or “species A”, averaging the kᵢ/m across I strands, to give k/m≡K/mI=1/I·kᵢ/m, testing k/m against avg(P_{L},P_{H}), some average (weighted, arithmetic, geometric, etc.) of P_{L} and P_{H}, rejecting H₁ (and accepting H₀) if k/m<avg(P_{L},P_{H}) and accepting H₁ (and rejecting H₀) if k/m≥avg(P_{L},P_{H}). The random variable K ≡Σkᵢ will have a binomial distribution with a mean of either (mI) P_{L} or (mI) P_{H} and a variance of either (mI) P_{L}(1−P_{L}) or (mI) P_{H}(1−P_{H}), both conditioned on whether the Pₙ=P_{L} or Pₙ=P_{H}; so, the random variable C_{A}≡k/m will have an approximately Gaussian distribution with mean μ_{(k/m)|Pn=PL}=P_{L} or μ_{(k/m)|Pn=PH}=P_{H} and standard deviation σ_{(k/m)|Pn=PL}={P_{L}(1−P_{L})}^{1/2}·1/(mI)^{1/2}≤0.5/(mI)^{1/2} or σ_{(k/m)|Pn=PH}={P_{H}(1−P_{H})}^{1/2}·1/(mI)^{1/2}≤0.5/(mI)^{1/2}.

If α and β are the probabilities of falsely rejecting the “accept” hypothesis given that is true (choosing H=H₁ when Pₙ=P_{H}) and falsely accepting the “accept” hypothesis given that it is false (choosing H=H₀ when Pₙ=P_{L}), respectively, then, as shown in Drake et al., 1988 (see p. 240-243 of ref.), the probability of forming a wrong estimate for Pₙ from C_{A,n} (that is, the probability that P\hat{ }ₙ≠Pₙ) is bounded such that min(α,β)≤Prob(incorrect conclusion)≤max(α,β). So, one may say Prob(incorrect conclusion)≈avg(α,β)=½(α+β).

If the pair of Gaussians are approximated by a pair of uniform distributions both of half-width a, with

$$a = \sqrt{3} σ \leq 0.866 / {( mI )}^{\frac{1}{2}} \leq 1 / {mI}^{1 / 2} ,$$

spaced a distance ΔP apart, on center, then α+β≈2(½−½ΔP/α) and ½(α+β)≈{1−ΔP/α}/2={1−ΔP (mI)^{1/2}}/2. So, there will be a negligible classification error whenever mI≥1/ΔP², and the maximum achievable spatial resolution for stimulus detection, as a fraction of the polymer length, will be given by m/N≥1/(I/N)·(1/ΔP²). By this formula, if ΔP=0.15, then to achieve a 3 bp resolution, one must average 15 strands.

### Discussion of the Examples

Summarily, targeted drug delivery has been an area of active investigation for many decades. Some approaches target cell-borne receptors; others use external stimuli such as heat or radio waves to drive spatially-localized release. As described herein, particles estimate their own location within the body by correlating their sensed fluid environment (e.g., temp., press., salinity, sugar, pH, etc.) against an embodied map and release on the basis of this estimate; the approach is related to terrain contour matching (TERCOM), a technique used in air navigation. Preliminarily explored particle concepts have included liposomes and proteins (bottom-up fab) and thin films (top-down fab). As envisioned, a mixture of drug-laden and empty permeable vehicles, each with a different environmental response, interconnect through a capacitive volume separated from the surroundings by a permeable film. In another envisioned approach, the monomer sequence of polypeptides or other polymers is selected to provide the greatest activity in preferred capillaries, the sequence of experienced environments affecting the conformation. In both, using item response theory, the mixture's or particle's composition is tailored to deliver a larger dose or greater activity to preferred capillaries. A chip concept that implements a microarray with a half-toned chemical library and material data drawn from conventional surgical analogs has also been considered as a basis for screening candidate compositions for the desired spatial sensitivity.

Overall, the presently disclosed subject matter relates in some embodiments to nanoparticles that record their experience in DNA and/or RNA, in some embodiments by estimating location within the body from vectors of sensed variables, and on the development of concepts for particles and chips. As such, in some embodiments the presently disclosed subject matter relates to nanoparticles that implement TERCOM- or DSMAC-like navigation in the body.

### REFERENCES

All references cited in the instant disclosure, including but not limited to all patents, patent applications and publications thereof, scientific journal articles, and database entries are incorporated herein by reference in their entireties to the extent that they supplement, explain, provide a background for, or teach methodology, techniques, and/or compositions employed herein.
Bae et al. (1991) “On-Off” thermocontrol of solute transport. I. Temperature dependence of swelling of N-isopropylacrylamide networks modified with hydrophobic components in water. Pharm. Res. 8(4):531-537.
Bae et al. (1991) “On-Off” Thermocontrol of Solute Transport. II. Solute Release from Thermosensitive Hydrogels. Pharm. Res. 8(5):624-628.
Bhan et al. (2019) Recording Temporal Data onto DNA with Minutes Resolution. bioRxiv preprint, 12 May 2019, ver., doi.org/10.1101/634790.
Chen et al. (1995) Novel Hydrogels of a Temperature-Sensitive Pluronic Grafted to a Bioadhesive Polyacrylic Acid Backbone for Vaginal Drug Delivery. Proceed. Intern. Symp. Control. Rel. Bioact. Mater. 22:167-168.
Chun & Kim (1996) A novel hydrogel-dispersed composite membrane of poly (N-isopropylacrylamide) in a gelatin matrix and its thermally actuated permeation of 4-acetaminophen. J. Controlled Release 38:39-47.
Constantine et al. (2016) Heterologously-expressed and Liposome-reconstituted Human Transient Receptor Potential Melastatin 4 Channel (TRPM4) is a Functional Tetramer. Scientific Reports (Nature) 6:19352.
D'Emanuele & Dinarvand (1995a) Preparation, characterisation, and drug release from thermoresponsive microspheres. Intl J. Pharm. 118:237-242.
Dinarvand & D'Emanuele (1995b) The use of thermoresponsive hydrogels for on-off release of molecules. J. Controlled Release 36:221-227.
Doelker (1993) Cellulose Derivatives. in Peppas and Langer (eds.), Biopolymers I, Springer-Verlag, Berlin.
GENBANK® Accession Nos. NP_060106.2, NP_060132.3, NP_060142.3, and NP_076985.4.
Gutowska et al. (1992) Heparin release from thermosensitive hydrogels. J. Controlled Release 22:95-104.
Harsh & Gehrke (1991) Controlling the swelling characteristics of temperature-sensitive cellulose ether hydrogels. J. Controlled Release 17:175-186.
Heller et al. (1988) Chemically Self-Regulated Drug Delivery Systems. in Polymers in Medicine III, Elsevier Science Publishers B.V., Amsterdam, pp. 175-188.
Hoffman (1987) Applications of Thermally Reversible Polymers and Hydrogels in Therapeutics and Diagnostics. in Third International Symposium on Recent Advances in Drug Delivery Systems, Salt Lake City, Utah, Feb. 24-27, 1987, pp. 297-305.
Hoffman (1988) Thermally Reversible Hydrogels Containing Biologically Active Species. in Migliaresi et al. (eds.), Polymers in Medicine III, Elsevier Science Publishers B.V., Amsterdam, pp. 161-167.
Hoffman et al. (1993) Characterizing Pore Sizes and Water ‘Structure’ in Stimuli-Responsive Hydrogels. Center for Bioengineering, Univ. of Washington, Seattle, Wash., p. 828.
Hsiao et al. (2017) Automated Modeling of Large-Scale Arterial Systems. in MIT Microsystems Technology Lab (MTL) Annual Research Report, page 21.
Johnston et al. (1992) Sustained delivery of interleukin-2 from a poloxamer 407 gel matrix following intraperitoneal injection in mice. Pharm. Res. 9(3):425-433.
Katono et al. (1991) Thermo-responsive swelling and drug release switching of interpenetrating polymer networks composed of poly(acrylamide-co-butyl methacrylate) and poly (acrylic acid). J. Controlled Release 16:215-228.
Kim et al. (1992) Hydrogels: swelling, drug loading, and release. Pharm. Res. 9(3):283-290.
Kobayashi et al. (1982) Diffusion of Beta-Lactam Antibiotics through Liposome Membranes Containing Purified Porins. Antimicrobial Agents and Chemotherapy 22(5):775-780.
Kono et al. (1994) Temperature-sensitive liposomes: liposomes bearing poly (N-isopropylacrylamide). J. Controlled Release 30:69-75.
Krissanaprasit et al. (2019) Genetically Encoded, Functional Single-Strand RNA Origami: Anticoagulant. Advanced Materials 31(21) 1808262.
Lassoued et al. (2017) A Hidden Markov Model for Route and Destination Prediction. 2017 IEEE 20th International Conference on Intelligent Transportation Systems (ITSC). DOI 10.1109/ITSC.2017.8317888.
Macdonald et al. (2002) Patch-clamp Experiments with Porins Extracted from a Marine Bacterium (Photobacterium profundum strain SS9) and Reconstituted in Liposomes. Cell Biochem Biophys 37:157-167.
Okano (1995) Molecular Design of Stimuli-Responsive Hydrogels for Temporal Controlled Drug Delivery. Proceed. Intern. Symp. Control. Rel. Bioact Mater. 22:111-112.
Okano et al. (1995) Temperature-responsive poly(N-isopropylacrylamide) as a modulator for alteration of hydrophilic/hydrophobic surface properties to control activation/inactivation of platelets. J. Controlled Release 36:125-133.
Paavola et al. (1995) Controlled release of lidocaine from injectable gels and efficacy in rat sciatic nerve block. Pharm. Res. 12(12):1997-2002.
Palasis & Gehrke (1992) Permeability of responsive poly (N-isopropylacrylamide) gel to solutes. J. Controlled Release 18:1-12.
PCT International Patent Application Publication Nos. WO 2000/066085, WO 2019/217601.
Peppas (1993) Fundamentals of pH- and Temperature-Sensitive Delivery Systems. in Gurny et al. (eds.), Pulsatile Drug Delivery, Wissenschaftliche Verlagsgesellschaft mbH, Stuttgart, pp. 41-55.
Salomon et al. (2020) A liposomal RNA vaccine inducing neoantigen-specific CD4⁺ T cells augments the antitumor activity of local radiotherapy in mice.
Oncoimmunology 9(1):1771925.
Tanner (2009) Recording Information into DNA. Ph.D. Dissertation, Duke University (available on the World Wide Web at hdl.handle.net/10161/1331).
Tanner et al. (2010) Experimental demonstration of lossy recording of information into DNA. in Proc. SPIE 7679, Micro- and Nanotechnology, Sensors, Systems, and Applications II, 77920; doi:10.1117/12.858775.
Tung (1994) Rheological behavior of poloxamer 407 aqueous solutions during sol-gel and dehydration processes. Intl J. Pharm. 107:85-90.
U.S. Patent Application Publication No. 2009/0275031.
U.S. Pat. Nos. 4,713,448; 4,913,743; 5,099,013; 5,128,326; 5,143,724; 5,246,698; 5,266,563; 5,399,351; 7,780,979.
Yoshida et al. (1994) Positive thermosensitive pulsatile drug release using negative thermosensitive hydrogels. J. Controlled Release 32:97-102.
Yu & Grainger (1993a) Novel Thermos-sensitive Amphiphilic Gels: Poly N-isopropylacrylamide-co-sodium acrylate-co-n-N-alkylacrylamide Network Synthesis and Physicochemical Characterization. Dept. of Chemical & Biological Sci., Oregon Graduate Institute of Science & Technology, Beaverton, Oreg., pp. 820-821.
Yu & Grainger (1993b) Thermo-sensitive Swelling Behavior in Crosslinked N-isopropylacrylamide Networks: Cationic, Anionic and Ampholytic Hydrogels. Dept. of Chemical & Biological Sci., Oregon Graduate Institute of Science & Technology, Beaverton, Oreg., pp. 829-830.
Zhou & Smid (1993) Physical Hydrogels of Associative Star Polymers. Polymer Research Institute, Dept. of Chemistry, College of Environmental Science and Forestry, State Univ. of New York, Syracuse, N.Y., pp. 822-823.

It will be understood that various details of the presently disclosed subject matter can be changed without departing from the scope of the presently disclosed subject matter. Furthermore, the foregoing description is for the purpose of illustration only, and not for the purpose of limitation.

1. A composition comprising, consisting essentially of, or consisting of:
(a) an active agent conjugated to a polymer strand, wherein the polymer strand comprises a monomer or unit, optionally a nucleotide sequence, that is modifiable to record one or more environmental conditions and/or a path in a volume experienced by the composition or that encodes a map for such a path for comparison to an actually-experienced path; and
(b) a carrier, wherein the carrier comprises a liposome, exosome, or other vehicle.

2. The composition of claim 1, wherein the nucleotide sequence is modified by regulating the addition and/or removal from the polymer strand to record the one or more environmental conditions and/or the path in the volume experienced at one or more of windows or probe tips of a micro-dialysis probed of any network or chain of micro-dialysis probes.

3. The composition of claim 1, wherein the composition further comprises a nuclease, a polymerase, or a terminal deoxynucleotidyl transferase (TdT) to append nucleotides to the polymer strand when experiencing the one or more environment conditions or path in the sequence of monomers of a polymer.

4. The composition of claim 3, wherein the polymer strand comprises a DNA strand and TdT is used to append nucleotides to the DNA strand when recording the one or more environmental conditions and/or the path in the volume.

5. The composition of claim 1, wherein the carrier is an environmentally sensitive liposome or exosome.

6. The composition of claim 1, wherein the composition makes use of the environmentally sensitive concentration, conformation, and/or level of activity of a catalyst to regulate appending monomers or units to the polymer strand when recording the one or more environmental conditions and/or the path in the volume.

7. An apparatus for testing and/or recording of one or more environmental conditions and/or traveled paths experienced by the composition of claim 1, the apparatus comprising, consisting essentially of, or consisting of an interconnected network of in-line or concentric micro-dialysis probes that is supplied for its flow with a formulation that generates a polymer comprising a monomer sequence that is a function of the one or more environmental conditions and/or traveled paths experienced by the composition through a network, wherein one or more windows or tips of the micro-dialysis probes are exposed to the one or more different environmental conditions and/or traveled paths.

8. A method for chemical recording of an environmental sequence experienced by a Fourier composition, the method comprising providing the composition of claim 1 and exposing the Fourier composition to one or more environmental conditions and/or paths in a volume experienced by the composition or that encodes a map for such a path for comparison to an actually-experienced path, whereby an environmental sequence experienced by the composition is recorded.

9. The method of claim 8, wherein the composition comprises, consists essentially of, or consists of a plurality of polymers and/or residuals thereof.

10. The method of claim 8, comprising removing monomers from the polymer or the plurality of polymers.

11. The method of claim 8, wherein the each member of the plurality of polymers comprises a sequence reflective of a basis function of a Fourier decomposition.

12. The method of claim 8, further comprising recording a Fourier spectrum of an environmental signal by removing monomers from the plurality of polymers in an environmentally sensitive manner.

13. The method of claim 8, where the polymer comprises, consists essentially of, or consists of a nucleotide sequence, optionally a DNA sequence.

14. The method of claim 8, where the polymer comprises, consists essentially of, or consists of an amino acid sequence.

15. A kit comprising the composition of claim 1 and at least one reagent required to perform a method for chemical recording of an environmental sequence experienced by the composition.

16. (canceled)

17. A method for consuming a monomer, block of monomers, or other such unit chain of a polymer in response to one or more environmental factors experienced by the monomer or the block of monomers or other such unit chain of a polymer to regulate a dosage of a therapeutic delivered to a target site, optionally a target site within an interconnected series of passages or volumes of an animal or a plant, optionally wherein the interconnected series of passages comprises a circulatory system of the animal or plant, the method comprising exposing the monomer, the block of monomers, or the other such unit chain of the polymer to the one or more environmental factors, wherein the exposing induces a modification of the monomer, the block of monomers, or the other such unit chain of the polymer.

18. The method of claim 17, wherein the polymer is an RNA molecule that comprises a coding region and a junk tail region that follows or precedes the coding region, and further wherein the junk tail region serves as an environmentally path-sensitive fuse that controls a dose of a therapeutic delivered to a target and the coding region encodes a therapeutic agent or functions as a template for synthesis of a therapeutic, optionally wherein the therapeutic agent is a polypeptide, a DNA sequence, a DNA sequence that encodes a therapeutic RNA, or a DNA sequence that encodes a therapeutic polypeptide or protein.

19. The method of claim 18, where the therapeutic agent is a therapeutic RNA, optionally an mRNA, an origami RNA, an interfering RNA, an RNA.

20. The method of claim 18, wherein the therapeutic RNA encodes an immunogenic peptide or polypeptide.

21. The method of claim 18, wherein the therapeutic agent is a polypeptide or a protein.

22. The method of claim 17, wherein the monomer, block of monomers, or other such unit chain of the polymer comprises a junk fuse that functions to modulate consumption of the monomer, block of monomers, or other such unit chain of the polymer in a manner that is selective for a pre-determined path or target.

23. The method of claim 17, wherein the coding region and junk fuse comprise, consist essentially of, or consist of a double-stranded DNA molecule, a single-stranded DNA molecule, and/or a DNA molecule that comprises one or more partially double-stranded regions and one or more single-stranded regions.

24. The method of claim 23, wherein at least one of the one or more partially double-stranded regions comprises an RNA polymerase transcriptional start sequence, at least one of the one or more single-stranded regions comprises a junk tail region.

25. The method of claim 17, wherein the coding region and the junk tail comprises an amino acid sequence, optionally a biologically active polypeptide, optionally a portion of which is therapeutically active, and further wherein the junk tail serves as the fuse, further optionally wherein the junk tail is associated with an enzyme that removes amino acids from the junk tail in an environmental path-sensitive manner.

26. A method for targeted therapy, the method comprising administering to an animal or plant the composition of claim 1, wherein the composition is encapsulated in a vehicle permits a reaction to take place while the composition traverses a circulatory system or some other such flow path or sequence of interconnected volumes of the animal or plant.

27. The method of claim 26, wherein the vehicle is a liposome, an exosome, or other carrier the comprises a lipid bilayer.

28. The method of claim 27, where the vehicle is a liposome comprising one or more pores in its lipid bilayer in order to make the liposome or the exosome permeable to the concentration of an ion and/or of one or more other environmental stimuli.

29. The method of claim 28, wherein the pores are formed from a naturally occurring porin, an engineered porin, or any combination thereof integrated into the lipid bilayer/

30. A method for using DNA as a template for producing an mRNA that comprises a coding region root or head and a junk region tail, wherein the junk region tail encodes a Fourier mode such that when the tail is attacked by a nuclease, optionally, an RNase, that removes units from the junk region tail with a removal rate that is sensitive to environment and to the type of unit being removed or the types of units in the vicinity of a removal site, and further wherein a mathematical vector dot product or inner product is accomplished and the time average removal rate, which is also the time until the coding region is broken, is represented as being sensitive to the inner product of the time-evolution of an environment and the spatial sequence of monomers in the junk region tail.

31. A method for synthesizing from a fused RNA via reverse transcription a sticky-end cDNA that is ligatable one or more nucleotide sequences strands that code for the synthesis of mRNA, the method comprising providing a nucleotide sequence encoding a sticky-end cDNA and a reverse transcriptase under conditions sufficient to synthesize a sticky-end cDNA from the fused RNA.

32. The method of claim 31, where polymers in general are used rather than specifically nucleic acid polymers.

33. The method of claim 32, wherein the tail removed from the RNA or the DNA in the environmentally sensitive fashion is synthesized using a TdT noisy chemical recording technique.

34. A kit comprising the composition of claim 1 and at least one reagent required to employ the composition to regulate dosing of the active agent or synthesis of a polypeptide encoded by the polymer strand.

35. A method for synthesizing a double-stranded DNA that encodes an mRNA comprising a coding head and a Fourier mode tail, the method comprising reverse transcribing one or more cDNA sticky-ended pieces of the mRNA, and thereafter ligating the sticky-ended pieces together to form the double-stranded DNA.

36. A formulation for chemical recording and DNA editing, the formulation comprising, consisting essentially of, or consisting of a DNA plasmid that comprises one or more genes required to synthesize an environmentally sensitive fusing of protein synthesis scheme described above and that also contains the genes required for expression of the various proteins and nucleic acid chains used in the reaction (in addition to or beyond those already provided in an appropriate cell).

37. The method of claim 17, further comprising adding a tail to DNA by TdT or another tailing reaction, optionally with sticky ends and T4 DNA ligase, after a coding region, wherein the coding region lacks a terminator between the region and the tail, such that transcription by RNA polymerase runs off the tail and the tail length thusly affects the rate of RNA polymerase recirculation and, therefore, the rate of transcription and translation, and, therefore an amount of polypeptide, or RNA, or reverse transcription product encoded by the coding region.

38. A chemical recording device or formulation that writes a polymer strand by regulating the addition of monomers to a polymer via a valve-like mechanism and that is used to record one or more environments experienced at one or more of windows or probe tips of a micro-dialysis probe or a network or chain of micro-dialysis probes.

39. The chemical recording device or formulation of claim 38, wherein the chemical recording device or formulation records a sensed environment or path in a sequence of monomers of a DNA strand.

40. The chemical recording device of claim 38, wherein the regulating makes use of TdT to append nucleotides to a DNA strand when recording the sensed environment or path in the sequence of monomers of a polymer.

41. The chemical recording device of claim 38, wherein the regulating makes use of environmentally-sensitive liposomes as a valve that regulates the process of appending monomers or units to a polymer or other chain or strand when recording the sensed environment or path in the sequence of monomers of a polymer or units of some chain.

42. The chemical recording device of claim 38, wherein the regulating makes use of an environmentally-sensitive concentration, conformation, and/or level of activity of a catalyst as a valve to regulate appending monomers or units to a polymer or other chain or strand when recording the sensed environment or path in the sequence of monomers of a polymer or units of some chain.

43. An apparatus for chemical recording of environments or traveled paths, wherein the apparatus comprises, consists essentially of, or consists of an interconnected network of in-line and/or concentric micro-dialysis probes that is supplied for its flow with a formulation that generates a polymer, a monomer sequence of which is a function of a path traveled through the network, the windows, or probe tips of the probes when exposed to different environments.

44. The apparatus of claim 43, wherein the apparatus is structured to record a sensed environment and/or path in nucleotides of a DNA strand, amino acids of a polypeptide, and/or monomers of a sugar polymer.

45-49. (canceled)

50. A method for randomly removing segments of DNA from a plasmid or piece of linear DNA while simultaneously adding cDNA synthesized through a reverse transcription process that is affected by experienced environmental path, wherein an approximate size of the DNA is relatively constant over time but its composition evolves with changing environment.

51. A method for employing one or more naturally occurring porins and/or engineered porins to make a liposome permeable to an ion, the concentration of which regulates a chemical recorder of genetic memory mechanism.

52. (canceled)

53. A drug delivery particle comprising the composition of claim 1.

54. A method for drug delivery comprising, consisting essentially of, or consisting of administering to a subject in need thereof an effective amount of the drug delivery particle of claim 53.

55. The method of claim 54, wherein the drug delivery particle employs correlation of a local environment in a circulatory system of the subject, or of a sequence of such environments, against a map embodied in its parameters in order to establish and respond to one or more locations of the drug delivery particle in the subject.

56. The method of claim 54, wherein the drug delivery particle employs correlation of a local environment in which it is located, or of a sequence of such environments, against a map embodied in its parameters in order to establish and respond to its location within the subject.

57. (canceled)

58. A microarray, bio-chip, bio-MEMS device, and/or well plate array structured to test a particle for sensitivity to environment or sequence of environments that the particle experiences.

59. The microarray, bio-chip, or bio-MEMS device of claim 58, wherein the microarray, bio-chip, or bio-MEMS device is structured to populate elements therein with material and/or chemical contents with material and/or chemical properties and other such metrics drawn from or patterned after a conventional surgical testing apparatus known as a ‘synthetic cadaver’, or any other such similar apparatus, or any database the contents of which are similar in concept to sampling the various environments of such an apparatus, or any database the contents of which are suitable for designing such an apparatus, or any part of such a database or any database that could form part of such a database, on a chip or similar apparatus.

60. The bio-chip or bio-MEMS device of claim 58, structured in the form of a synthetic cadaver on a chip.

61. A particle comprising, consisting essentially of, or consisting of a polypeptide or long-chain polymer or other strand of material with an environmentally-sensitive conformation conjugated to or associated with a drug moiety or active region that activates under different environmental conditions or sequences of environmental conditions.

62. The particle of claim 61, wherein the conformation depends on the sequence of environmental conditions.

63. The particle of claim 61, wherein vehicles containing or bearing one or more particles with TERCOM-like or DSMAC-like behavior are encased by a film such as a porous lipid bilayer or some other material film.

64. The particle of claim 63, wherein the vehicles are encased by a film such as a porous lipid bilayer or some other material film.

65. The particle of claim 63, wherein the vehicles are tethered or connected but not encased by any additional material, meaningful resistance to diffusion, to the extent it exists, occurring simply due to the radial nature of diffusion out of an interstitial volume.

66. The particle of claim 63, wherein the interstitial volume serves as a capacitive layer that averages the rate of release from the individual particle types.


SEQUENCE LISTING (2 sequences)

SEQ ID NO: 1 - DNA, 22 bp, Artificial Sequence
  Artificially synthesized oligonucleotide
acactgacgacatggttctaca

SEQ ID NO: 2 - DNA, 22 bp, Artificial Sequence
  Artificially synthesized oligonucleotide
agaccaagtctctgctaccgta